SEQUENCE LISTING (93 sequences)

SEQ ID NO: 1 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDECLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 3 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVCTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVCEQDSKDSTYSLSSTLTLSKADYEKHKVYACEVTHQGLSSPVTKSFNRGES

SEQ ID NO: 5 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, synthetic construct; identical to SEQ ID NO: 4

SEQ ID NO: 7 - protein, 450 aa, synthetic construct; identical to SEQ ID NO: 5

SEQ ID NO: 8 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 9 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct; identical to SEQ ID NO: 8

SEQ ID NO: 11 - protein, 450 aa, synthetic construct; identical to SEQ ID NO: 9

SEQ ID NO: 12 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGES

SEQ ID NO: 13 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 15 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKCTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVCIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 17 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTCGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 19 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct; identical to SEQ ID NO: 8

SEQ ID NO: 21 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGCAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, synthetic construct; identical to SEQ ID NO: 16

SEQ ID NO: 23 - protein, 449 aa, synthetic construct
EVQLVESGGGLVQPGGSLRLSCAASGFTFTDYTMDWVRKAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 214 aa, synthetic construct
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVCEQDSKDSTYSLSSTLTLSKADYEKHKVYACEVTHQGLSSPVTKSFNRGES

SEQ ID NO: 25 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFCPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 27 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSCKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, synthetic construct; identical to SEQ ID NO: 26

SEQ ID NO: 29 - protein, 450 aa, synthetic construct; identical to SEQ ID NO: 27

SEQ ID NO: 30 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 31 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSCSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFCFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 33 - protein, 450 aa, synthetic construct; identical to SEQ ID NO: 31

SEQ ID NO: 34 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTCSFNRGES

SEQ ID NO: 35 - protein, 450 aa, synthetic construct; identical to SEQ ID NO: 31

SEQ ID NO: 36 - protein, 214 aa, synthetic construct; identical to SEQ ID NO: 30

SEQ ID NO: 37 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTACLGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct; identical to SEQ ID NO: 16

SEQ ID NO: 39 - protein, 449 aa, synthetic construct; identical to SEQ ID NO: 23

SEQ ID NO: 40 - protein, 214 aa, synthetic construct
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 41 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLACSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, synthetic construct; identical to SEQ ID NO: 14

SEQ ID NO: 43 - protein, 450 aa, synthetic construct; identical to SEQ ID NO: 15

SEQ ID NO: 44 - protein, 214 aa, synthetic construct; identical to SEQ ID NO: 14

SEQ ID NO: 45 - protein, 450 aa, synthetic construct; identical to SEQ ID NO: 5

SEQ ID NO: 46 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYCLSSTLTLSKADYEKHKVYACEVTHQGLSSPVTKSFNRGES

SEQ ID NO: 47 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLCSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLCLSKADYEKHKVYACEVTHQGLSSPVTKSFNRGES

SEQ ID NO: 49 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYCLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSCLTLSKADYEKHKVYACEVTHQGLSSPVTKSFNRGES

SEQ ID NO: 51 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLCSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 214 aa, synthetic construct; identical to SEQ ID NO: 50

SEQ ID NO: 53 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 213 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 55 - protein, 449 aa, synthetic construct
EVQLVESGGGLVQPGGSLRLSCAASGFTFTDYTMDWVRKAPGKGLEWVADVNPNSGGSIYNQRFKGRFTLSVDRSKNTLYLQMNSLRAEDTAVYYCARNLGPSFYFDYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - protein, 214 aa, synthetic construct
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIKRTVAAPSVFIFPPSDECLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 57 - protein, 450 aa, synthetic construct
EVQLVESGGGLVQPGGSLRLSCAASGFNIKDTYIHWVRKAPGKGLEWVARIYPTNGYTRYADSVKGRFTISADTSKNTAYLQMNSLRAEDTAVYYCSRWGGDGFYAMDYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGQGTKVEIKRTVAAPSVFIFPPSDECLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 59 - protein, 449 aa, synthetic construct
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQADLPAFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDECLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 63 - protein, 447 aa, synthetic construct
QVQLVQSGAEVKKPGSSVKVSCKASGYTFSDYVISWVRQAPGQGLEWMGEIYPRGGIIHYNEKFKARVTITADKSTSTAYMELSSLRSEDTAVYYCARLWDWYFDVWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 220 aa, synthetic construct
EIVLTQSPGTLSLSPGERATLSCRSSQSLVHSQGITYLDWYQQKPGQAPRLLIYKVSNRFSGIPDRFSGSGSGTDFTLTISRLEPEDFAVYYCSQGTHVPPWTFGGGTKVEIKRTVAAPSVFIFPPSDECLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 65 - protein, 447 aa, synthetic construct
QVQLVQSGAEVKKPGSSVKVSCKASGYTFSDYVISWVRQAPGQGLEWMGEIYPRGGIIHYNEKFKARVTITADKSTSTAYMELSSLRSEDTAVYYCARLWDWYFDVWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 220 aa, synthetic construct; identical to SEQ ID NO: 64

SEQ ID NO: 67 - protein, 447 aa, synthetic construct
QVQLVQSGAEVKKPGSSVKVSCKASGYTFSDYVISWVRQAPGQGLEWMGEIYPRGGIIHYNEKFKARVTITADKSTSTAYMELSSLRSEDTAVYYCARLWDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - protein, 219 aa, synthetic construct
EIVLTQSPGTLSLSPGERATLSCRSSQSLVHSQGITYLDWYQQKPGQAPRLLIYKVSNRFSGIPDRFSGSGSGTDFTLTISRLEPEDFAVYYCSQGTHVPPWTFGGGTKVEIKGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 69 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 449 aa, synthetic construct
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 120 aa, synthetic construct [sequence omitted]

SEQ ID NO: 73 - protein, 107 aa, synthetic construct [sequence omitted]

SEQ ID NO: 74 - protein, 450 aa, synthetic construct; identical to SEQ ID NO: 57

SEQ ID NO: 75 - protein, 107 aa, synthetic construct
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGQGTKVEIK

SEQ ID NO: 76 - protein, 119 aa, synthetic construct
EVQLVESGGGLVQPGGSLRLSCAASGFTFTDYTMDWVRKAPGKGLEWVADVNPNSGGSIYNQRFKGRFTLSVDRSKNTLYLQMNSLRAEDTAVYYCARNLGPSFYFDYWGQGTLVTVSS

SEQ ID NO: 77 - protein, 107 aa, synthetic construct
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIK

SEQ ID NO: 78 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 79 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 80 - protein, 117 aa, synthetic construct
QVQLVQSGAEVKKPGSSVKVSCKASGYTFSDYVISWVRQAPGQGLEWMGEIYPRGGIIHYNEKFKARVTITADKSTSTAYMELSSLRSEDTAVYYCARLWDWYFDVWGQGTLVTVSS

SEQ ID NO: 81 - protein, 113 aa, synthetic construct
EIVLTQSPGTLSLSPGERATLSCRSSQSLVHSQGITYLDWYQQKPGQAPRLLIYKVSNRFSGIPDRFSGSGSGTDFTLTISRLEPEDFAVYYCSQGTHVPPWTFGGGTKVEIK

SEQ ID NO: 82 - protein, 449 aa, synthetic construct
EVQLVESGGGLVQPGGSLRLSCAASGFTFTDYTMDWVRKAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKCTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 214 aa, synthetic construct
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIKRTVAAPSVCIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 84 - protein, 449 aa, synthetic construct
EVQLVESGGGLVQPGGSLRLSCAASGFTFTDYTMDWVRKAPGKGLEWVADVNPNSGGSIYNQRFKGRFTLSVDRSKNTLYLQMNSLRAEDTAVYYCARNLGPSFYFDYWGQGTLVTVSSASTKGPSVFPLACSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 85 - protein, 214 aa, synthetic construct
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 86 - protein, 450 aa, synthetic construct; identical to SEQ ID NO: 15

SEQ ID NO: 87 - protein, 213 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGQPKAAPSVCLFPPSSEELQANKATLVCLISDFYPGAVTVAWKADSSPVKAGVETTTPSKQSNNKYAASSYLSLTPEQWKSHRSYSCQVTHEGSTVEKTVAPTESS

SEQ ID NO: 88 - protein, 107 aa, synthetic construct [sequence omitted]

SEQ ID NO: 89 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 90 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 91 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 224 aa, synthetic construct
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 330 aa, synthetic construct [sequence omitted]

(19) United States
(12) Patent Application Publication
(10) Pub. No.: US 2026/0295063 A1
(43) Pub. Date: Oct. 1, 2026

(54) MODIFIED ANTIBODIES, ANTIBODY FRAGMENTS THEREOF, AND ANTIBODY-DRUG CONJUGATES

(71) Applicant: INNOVENT BIOLOGICS (SUZHOU) CO., LTD., Suzhou, Jiangsu (CN)

(72) Inventors: Xiao ZHANG, Suzhou, Jiangsu (CN); Xiao YANG, Suzhou, Jiangsu (CN); Chenchen ZHU, Suzhou, Jiangsu (CN); Changshou GAO, Suzhou, Jiangsu (CN); Hassan ISSAFRAS, Suzhou, Jiangsu (CN); Cuihua GAO, Suzhou, Jiangsu (CN)

(73) Assignee: INNOVENT BIOLOGICS (SUZHOU) CO., LTD., Suzhou, Jiangsu (CN)

(21) Appl. No.: 19/479,583

(22) PCT Filed: Apr. 29, 2024
(86) PCT No.: PCT/CN2024/090596

§ 371 Date: Oct. 28, 2025

(30) Foreign Application Priority Data
Apr. 29, 2023 (WO) PCT/CN2023/091912
Apr. 26, 2024 (CN) 202410509547.8

(51) Int. Cl. A61K 47/68; A61P 35/00; C07K 16/28; C07K 16/30; C07K 16/32

(52) U.S. Cl. CPC A61K 47/68037; A61K 47/68031; A61K 47/6849; A61K 47/6855; A61K 47/6889; A61P 35/00; C07K 16/2827; C07K 16/30; C07K 16/32

(57) ABSTRACT

Provided are modified antibodies, antibody fragments thereof, and antibody-drug conjugates (ADCs) where one or more of the heavy-light chain disulfide bonds are moved to a solvent inaccessible region.

### INCORPORATION-BY-REFERENCE OF SEQUENCE LISTING

The application contains a sequence listing which has been submitted electronically in XML format and is hereby incorporated by reference in its entirety. Said XML copy, created on Oct. 21, 2025, is named 11275-013470-US0_SL.xml and is 109,027 bytes in size.

### TECHNICAL FIELD

The disclosure relates to modified antibodies, antibody fragments thereof, and antibody-drug conjugates (ADCs) where one or more of the heavy-light chain disulfide bonds are moved to a solvent inaccessible region.

### BACKGROUND

Antibody-drug conjugates (ADCs) have emerged as an important class of biologics for targeted treatment of cancers. So far, 14 ADCs have been approved by the US FDA. ADCs are complex molecules composed of an antibody linked to biologically active cytotoxic drugs. Conventional strategies for drug attachment (i.e. lysine- and interchain cysteine-based conjugation to achieve an average drug-to-antibody ratio (DAR) of target level) suffer from heterogeneity of conjugation sites and attached drugs. Progress in the development of site-specific conjugation has enabled the preparation of homogeneous ADCs. However, most of the strategies require re-engineering of the antibody sequence, multistep operation and costly substrates, thus limit the use of these methods in industry.

Thus, there is a need for modified antibodies and antibody fragments thereof that facilitate the preparation of homogeneous ADCs.

### SUMMARY

The present disclosure relates to modified antibodies, antibody fragments thereof, and ADCs where one or more of the heavy-light chain disulfide bonds are moved to a solvent inaccessible region. In some embodiments, the modified antibodies and antibody fragments thereof can facilitate the preparation of homogeneous ADCs.

In some embodiments, one or more of the heavy-light chain disulfide bonds are moved to a solvent inaccessible region, so that the new disulfide bonds are not opened by reducing agents at typical reducing conditions (e.g., excess TCEP). In some embodiments, a non-native disulfide bond is formed at the positions of a heavy chain amino acid residue and a light chain amino acid residue. In some embodiments, a solvent inaccessible region is a region where these disulfide bonds will not be opened by the typical reducing conditions. In some embodiments, the relative solvent accessible surface area (SASA) of the side chains of the candidate amino acid pairs in a structure of a Fab fragment are calculated. In some embodiments, a disulfide bond is categorized as “solvent accessible” if (1) the sum of the relative SASA of the two amino acid side chains is greater than 65%. In some embodiments, a disulfide bond is categorized as “solvent inaccessible” if (1) the sum of the relative SASA of the two amino acid side chains is below 65%. In some embodiments, the sum of the relative SASA of the two amino acid side chains is below 65%. In some embodiments, the relative SASA of the first amino acid side chain is above 40% and the relative SASA of the second amino acid side chain is below 10%. In some embodiments, the relative SASA of the first amino acid side chain is above 30% and the relative SASA of the second amino acid side chain is below 15%. In some embodiments, the relative SASA of each of the two amino acid side chains is below 20%, 25%, 30%, 35%, 40%, or 45%.

In one aspect, the disclosure is related to an antibody-drug conjugate (ADC) or pharmaceutically acceptable salt or solvate thereof comprising a payload and an antibody or antibody fragment (e.g., antigen-binding fragment) thereof, wherein the antibody or antibody fragment thereof comprises a non-native disulfide bond that is formed at the positions of a heavy chain amino acid residue and a light chain amino acid residue, wherein the sum of (1) the relative solvent accessible surface area (SASA) of the heavy chain amino acid residue side chain and (2) the relative SASA of the light chain amino acid residue side chain is below 65%, 60%, or 55%, wherein the antibody or antibody fragment thereof does not have a disulfide bond between the amino acid residue at position 220 of the heavy chain constant region and the amino acid residue at position 214 of the light chain constant region, wherein the amino acid position is according to EU numbering.

In some embodiments, the relative SASA of the heavy chain amino acid side chain is above 40% and the relative SASA of the light chain amino acid side chain is below 10%; or the relative SASA of the light chain amino acid side chain is above 40% and the relative SASA of the heavy chain amino acid side chain is below 10%.

In some embodiments, the relative SASA of the heavy chain amino acid side chain is above 30% and the relative SASA of the light chain amino acid side chain is below 15%; or the relative SASA of the light chain amino acid side chain is above 30% and the relative SASA of the heavy chain amino acid side chain is below 15%.

In some embodiments, the relative SASA of each of the heavy chain and light chain amino acid side chains is below 20%, 25%, 30%, 35%, 40%, or 45%.

In some embodiments, the amino acid residue at position 220 of a heavy chain constant region is a non-cysteine residue; or the amino acid residue at position 214 of a light chain constant region is a non-cysteine residue, wherein the amino acid position is according to EU numbering.

In some embodiments, the non-native disulfide bond is resistant to the reduction by reducing agent such as tris(2-carboxyethyl) phosphine hydrochloride (TCEP) and/or dithiothreitol (DTT).

In some embodiments, more than 90% or 95% of the non-native disulfide bonds remain intact when the antibody or antibody fragment thereof is reduced by 10-30 equivalents of TCEP per single antibody molecule.

In some embodiments, the concentration of the antibody or antibody fragment thereof is between 0.1-50 mg/ml during the reduction reaction.

In some embodiments, the non-native disulfide bond remains stable under reducing conditions that can reduce more than 90% of hinge region disulfide bonds of the antibody or antibody fragment.

In some embodiments, at least one non-cysteine amino acid residue in the heavy chain is mutated to a cysteine residue.

In some embodiments, the non-cysteine amino acid residue is located at a position selected from the group consisting of: 126, 168, 170, 173, 134, 133, 130, and 141, wherein the amino acid position is according to EU numbering.

In some embodiments, the antibody or antibody fragment thereof comprises one of the following mutations in the heavy chain constant region: F126C, H168C, F170C, V173C, S134C, K133C, P130C, and A141C.

In some embodiments, the antibody or antibody fragment thereof comprises two heavy chains, each of which comprises one more of the following mutations in the heavy chain constant region: F126C, H168C, F170C, V173C, S134C, K133C, P130C, and A141C.

In some embodiments, the antibody or antibody fragment thereof comprises two heavy chains, and only one heavy chain comprises one of the following mutations in the heavy chain constant region: F126C, H168C, F170C, V173C, S134C, K133C, P130C, and A141C.

In some embodiments, at least one non-cysteine amino acid in the light chain constant region of the antibody or antibody fragment thereof is mutated to a cysteine residue.

In some embodiments, the at least one non-cysteine amino acid is located at a position selected from the group consisting of: 124, 164, 162, 116, 209, 117, and 118.

In some embodiments, the antibody or antibody fragment thereof comprises one or more of the following mutations in the light chain constant region: Q124C, T164C, S162C, F116C, F209C, I117C, and F118C.

In some embodiments, the antibody or antibody fragment thereof comprises two light chains, each of which comprises one or more of the following mutations in the light chain constant region: Q124C, T164C, S162C, F116C, F209C, I117C, and F118C.

In some embodiments, the antibody or antibody fragment thereof comprises two light chains, and only one light chain comprises one or more of the following mutations in the light chain constant region: Q124C, T164C, S162C, F116C, F209C, I117C, and F118C.

In some embodiments, the antibody or antibody fragment thereof comprises one or more of the below sets of mutations:
F126C in the heavy chain and Q124C in the light chain;
H168C in the heavy chain and T164C in the light chain;
F170C in the heavy chain and T164C in the light chain;
F170C in the heavy chain and S162C in the light chain;
V173C in the heavy chain and S162C in the light chain;
S134C in the heavy chain and F116C in the light chain;
K133C in the heavy chain and F209C in the light chain;
K133C in the heavy chain and I117C in the light chain;
P130C in the heavy chain and F118C in the light chain; and
A141C in the heavy chain and F116C in the light chain.

In some embodiments, the antibody or antibody fragment thereof comprises two pairs of heavy and light chains, of which only one pair of heavy and light chains comprise one of the below sets of mutations:
F126C in the heavy chain and Q124C in the light chain;
H168C in the heavy chain and T164C in the light chain;
F170C in the heavy chain and T164C in the light chain;
F170C in the heavy chain and S162C in the light chain;
V173C in the heavy chain and S162C in the light chain;
S134C in the heavy chain and F116C in the light chain;
K133C in the heavy chain and F209C in the light chain;
K133C in the heavy chain and I117C in the light chain;
P130C in the heavy chain and F118C in the light chain; and
A141C in the heavy chain and F116C in the light chain.

In some embodiments, the antibody or antibody fragment thereof comprises two pairs of heavy and light chains, of which each pair of heavy and light chains comprise one or more of the below sets of mutations:
F126C in the heavy chain and Q124C in the light chain;
H168C in the heavy chain and T164C in the light chain;
F170C in the heavy chain and T164C in the light chain;
F170C in the heavy chain and S162C in the light chain;
V173C in the heavy chain and S162C in the light chain;
S134C in the heavy chain and F116C in the light chain;
K133C in the heavy chain and F209C in the light chain;
K133C in the heavy chain and I117C in the light chain;
P130C in the heavy chain and F118C in the light chain; and
A141C in the heavy chain and F116C in the light chain.

In some embodiments, the antibody or antibody fragment thereof comprises two pairs of heavy and light chains, wherein each heavy chain comprises F126C and each light chain comprises Q124C.

In some embodiments, the antibody or antibody fragment thereof comprises two pairs of heavy and light chains, wherein each heavy chain comprises H168C and each light chain comprises T164C.

In some embodiments, the antibody or antibody fragment thereof comprises two pairs of heavy and light chains, wherein each heavy chain comprises F170C and each light chain comprises T164C.

In some embodiments, the antibody or antibody fragment thereof comprises two pairs of heavy and light chains, wherein each heavy chain comprises F170C and each light chain comprises S162C.

In some embodiments, the antibody or antibody fragment thereof comprises two pairs of heavy and light chains, wherein each heavy chain comprises V173C and each light chain comprises S162C.

In some embodiments, the antibody or antibody fragment thereof comprises two pairs of heavy and light chains, wherein each heavy chain comprises S134C and each light chain comprises F116C.

In some embodiments, the antibody or antibody fragment thereof comprises two pairs of heavy and light chains, wherein each heavy chain comprises K133C and each light chain comprises F209C.

In some embodiments, the antibody or antibody fragment thereof comprises two pairs of heavy and light chains, wherein each heavy chain comprises K133C and each light chain comprises I117C.

In some embodiments, the antibody or antibody fragment thereof comprises two pairs of heavy and light chains, wherein each heavy chain comprises P130C and each light chain comprises F118C.

In some embodiments, the antibody or antibody fragment thereof comprises two pairs of heavy and light chains, wherein each heavy chain comprises A141C and each light chain comprises F116C.

In one aspect, the disclosure is related to an ADC comprising a non-native disulfide bond that is formed at the positions of a heavy chain amino acid residue and a light chain amino acid residue, wherein the antibody or antibody fragment thereof does not have a disulfide bond between the amino acid residue at position 220 of the heavy chain constant region and the amino acid residue at position 214 of the light chain constant region, wherein when the antibody or antibody fragment thereof is exposed to a reducing condition that reduces more than 90% or 95% of the hinge disulfide bonds, more than 90% or 95% of the non-native disulfide bonds remain intact.

In some embodiments, the antibody or antibody fragment thereof comprises one or more of the below sets of heavy chain mutations:
F126C and C220S;
H168C and C220S;
F170C and C220S;
V173C and C220S;
S134C and C220S;
K133C and C220S;
P130C and C220S; and
A141C and C220S.

In some embodiments, the antibody or antibody fragment thereof comprises one or more of the below sets of light chain mutations:
Q124C and C214S;
T164C and C214S;
S162C and C214S;
F116C and C214S;
F209C and C214S;
I117C and C214S; and
F118C and C214S.

In some embodiments, the antibody or antibody fragment thereof comprises one or more of the below sets of mutations:
F126C and C220S in the heavy chain and Q124C and C214S in the light chain;
H168C and C220S in the heavy chain and T164C and C214S in the light chain;
F170C and C220S in the heavy chain and T164C and C214S in the light chain;
F170C and C220S in the heavy chain and S162C and C214S in the light chain;
V173C and C220S in the heavy chain and S162C and C214S in the light chain;
S134C and C220S in the heavy chain and F116C and C214S in the light chain;
K133C and C220S in the heavy chain and F209C and C214S in the light chain;
K133C and C220S in the heavy chain and I117C and C214S in the light chain;
P130C and C220S in the heavy chain and F118C and C214S in the light chain; and
A141C and C220S in the heavy chain and F116C and C214S in the light chain.

In some embodiments, the antibody or antibody fragment thereof covers a Fab fragment, a (Fab) 2 fragment, a one-armed antibody, and/or a multi-specific antibody (e.g., a bispecific antibody).

In some embodiments, the antibody or antibody fragment thereof is a human or humanized antibody or antibody fragment thereof, a Fab fragment, a (Fab) 2 fragment, a one-armed antibody, and/or a multi-specific antibody (e.g., a bispecific antibody).

In some embodiments, the antibody or antibody fragment thereof is a human IgG1, IgG2, IgG4 antibody or antibody fragment thereof.

In some embodiments, the antibody or antibody fragment thereof comprises a fragment crystallizable region (Fc region).

In some embodiments, the heavy chain constant region is a constant region of (human) IgG1, IgG2, IgG3 or IgG4, e.g., a constant region of IgG1. In some embodiments, the IgG1 heavy chain constant region suitable for the antibodies or ADCs of the invention comprises or consists of:
(i) an amino acid sequence that has at least 85%, 90%, 91%, 92%, 93%, 94%, 95%, 96%, 97%, 98% or 99% identity to the amino acid sequence of SEQ ID NO: 93;
(ii) an amino acid sequence of SEQ ID NO: 93; or
(iii) an amino acid sequence having one or more (preferably no more than 10, more preferably no more than 5, 4, 3, 2, 1) amino acid changes (preferably amino acid substitutions, more preferably amino acid conservative substitutions) compared to the amino acid sequence of SEQ ID NO: 93.

In some embodiments, the light chain constant region is a (human) Kappa or Lambda constant region. In some embodiments, the human Kappa constant region comprises or consists of:
(i) an amino acid sequence that has at least 85%, 90%, 91%, 92%, 93%, 94%, 95%, 96%, 97%, 98% or 99% identity to the amino acid sequence of SEQ ID NO: 88;
(ii) an amino acid sequence of SEQ ID NO:88; or
(iii) an amino acid sequence having one or more (preferably no more than 10, more preferably no more than 5, 4, 3, 2, 1) amino acid changes (preferably amino acid substitutions, more preferably amino acid conservative substitutions) compared to the amino acid sequence of SEQ ID NO: 88.

In some embodiments, the human Lambda constant region comprises or consists of:
(i) an amino acid sequence that has at least 85%, 90%, 91%, 92%, 93%, 94%, 95%, 96%, 97%, 98% or 99% identity to the amino acid sequence of SEQ ID NO:89;
(ii) an amino acid sequence of SEQ ID NO:89; or
(iii) an amino acid sequence having one or more (preferably no more than 10, more preferably no more than 5, 4, 3, 2, 1) amino acid changes (preferably amino acid substitution, more preferably amino acid conservative substitution) compared to the amino acid sequence of SEQ ID NO: 89.

In some embodiments, the payload is a cytotoxic or cytostatic agent, biologically active proteins, synthetic polymers, enzymes, nucleic acids (e.g., DNA, or RNA) and fragments thereof.

In some embodiments, the drug-to-antibody ratio (DAR) is between 3.74 and 4.

In some embodiments, the drug-to-antibody ratio (DAR) is between 5.70 and 6.

In some embodiments, the payload is covalently bound to the antibody or antibody fragment thereof via a thiol-based conjugation.

In one aspect, the disclosure is related to an antibody or antibody fragment thereof comprising a non-native disulfide bond that is formed at the positions of a heavy chain amino acid residue and a light chain amino acid residue, wherein the sum of (1) the relative solvent accessible surface area (SASA) of the heavy chain amino acid residue side chain and (2) the relative SASA of the light chain amino acid residue side chain is below 65%, 60%, or 55%, wherein the antibody or antibody fragment thereof does not have a disulfide bond between the amino acid residue at position 220 of the heavy chain constant region and the amino acid residue at position 214 of the light chain constant region, wherein the amino acid position is according to EU numbering.

In some embodiments, the relative SASA of the heavy chain amino acid side chain is above 40% and the relative SASA of the light chain amino acid side chain is below 10%; or the relative SASA of the light chain amino acid side chain is above 40% and the relative SASA of the heavy chain amino acid side chain is below 10%.

In some embodiments, the relative SASA of the heavy chain amino acid side chain is above 30% and the relative SASA of the light chain amino acid side chain is below 15%; the relative SASA of the light chain amino acid side chain is above 30% and the relative SASA of the heavy chain amino acid side chain is below 15%.

In some embodiments, the relative SASA of each of the heavy chain and light chain amino acid side chains is below 20%, 25%, 30%, 35%, 40%, or 45%.

In one aspect, the disclosure is related to an antibody-drug conjugate comprising the antibody or antibody fragment thereof described herein, covalently bound to a payload.

In one aspect, the disclosure is related to a nucleic acid encoding a light chain or a heavy chain of the antibody or antibody fragment thereof described herein.

In one aspect, the disclosure is related to a vector comprising the nucleic acid described herein, wherein the nucleic acid is operably linked to a promoter.

In one aspect, the disclosure is related to a cell comprising the nucleic acid described herein or the vector described herein.

In one aspect, the disclosure is related to a method of producing an antibody or an antibody fragment thereof, the method comprising culturing the cell described herein under conditions sufficient for the cell to produce the antibody or the antibody fragment; and collecting the antibody or the antibody fragment produced by the cell.

In one aspect, the disclosure is related to a method of preparing an antibody-drug conjugate (ADC), comprising the following steps:
(a) incubating the antibody or antibody fragment thereof described herein with a reductant to reduce native interchain disulfide bonds within the antibody or antibody fragment thereof to generate reduced thiol groups;
(b) introducing an excess amount of a payload having a reactive group to react with the reduced thiol groups generated in step (a).

In some embodiments, the method further comprises: (c) adding an effective amount of N-acetylcysteine to quench the excess payload, and then recovering the resultant antibody-drug conjugate.

In some embodiments, the payload comprises a maleimide group, thiol, monobromomaleimide, dibromomaleimide, Iodoacetamides, N-Methyl-Nphenylvinylsulfonamide, Divinylpyrimidine, etc.

In some embodiments, the payload is linked to the antibody or antibody fragment thereof via a thiol-based conjugation.

In some embodiments, at least 50%, 60%, 70%, 80%, or 90% of the resulting ADC products have a drug-to-antibody ratio (DAR) of about 4.

In some embodiments, at least 50%, 60%, 70%, 80%, or 90% of the resulting ADC products have a drug-to-antibody ratio (DAR) of about 6.

In one aspect, the disclosure is related to a method of making a modified antibody or antibody fragment thereof, the method comprising:
(a) providing an antibody or antibody fragment thereof, wherein the antibody or antibody fragment thereof does not have a disulfide bond between the amino acid residue at position 220 of the heavy chain constant region and the amino acid residue at position 214 of the light chain constant region, wherein the amino acid position is according to EU numbering;
(b) identifying a heavy chain amino acid residue in the heavy chain constant region (CH1) and a light chain amino acid residue in the light chain constant region (CL), wherein the sum of (1) the relative solvent accessible surface area (SASA) of the heavy chain amino acid residue side chain and (2) the relative SASA of the light chain amino acid residue side chain is below 65%, 60%, or 55%;
(c) substituting each of the identified heavy chain amino acid residue and light chain amino acid residue with a cysteine residue, wherein a non-native disulfide bond is formed between the two cysteine residues.

In some embodiments, the relative SASA of the heavy chain amino acid side chain is above 40% and the relative SASA of the light chain amino acid side chain is below 10%; or the relative SASA of the light chain amino acid side chain is above 40% and the relative SASA of the heavy chain amino acid side chain is below 10%.

In some embodiments, the relative SASA of the heavy chain amino acid side chain is above 30% and the relative SASA of the light chain amino acid side chain is below 15%; or the relative SASA of the light chain amino acid side chain is above 30% and the relative SASA of the heavy chain amino acid side chain is below 15%.

In some embodiments, the relative SASA of each of the heavy chain and light chain amino acid side chains is below 20%, 25%, 30%, 35%, 40%, or 45%.

In some embodiments, the method further comprises adding a payload to the modified antibody or antibody fragment thereof.

In one aspect, the disclosure is related to a method of treating a subject having cancer, the method comprising administering a therapeutically effective amount of a composition comprising the ADC described herein, to the subject.

In some embodiments, the subject has a solid tumor cancer.

In some embodiments, the cancer is breast cancer, lung cancer, pancreatic cancer, melanoma, oral cancer, mesothelioma, ovarian cancer, colorectal cancer, gastric cancer, cervical cancer, brain cancer, skin cancer, multiple myeloma, lymphoma, epithelial neoplasms, soft tissue sarcoma, esophageal cancers, or CNS tumors.

In one aspect, the disclosure is related to a method of decreasing the rate of tumor growth, the method comprising contacting a tumor cell with an effective amount of a composition comprising the ADC or pharmaceutically acceptable salt or solvate thereof described herein.

In one aspect, the disclosure is related to a method of killing a tumor cell, the method comprising contacting a tumor cell with an effective amount of a composition comprising the ADC or pharmaceutically acceptable salt or solvate thereof described herein.

In one aspect, the disclosure is related to a pharmaceutical composition comprising the ADC or pharmaceutically acceptable salt or solvate thereof described herein, and a pharmaceutically acceptable carrier.

In some embodiments, the ADC comprises two pairs of heavy and light chains, wherein each heavy chain comprises mutations F126C and C220S and each light chain comprises mutations Q124C and C214S.

In some embodiments, the ADC comprises two pairs of heavy and light chains, wherein each heavy chain comprises mutations H168C and C220S and each light chain comprises mutations T164C and C214S.

In some embodiments, the ADC comprises two pairs of heavy and light chains, wherein each heavy chain comprises mutations F170C and C220S and each light chain comprises T164C and C214S.

In some embodiments, the ADC comprises two pairs of heavy and light chains, wherein each heavy chain comprises mutations F170C and C220S and each light chain comprises mutations S162C and C214S.

In some embodiments, the ADC comprises two pairs of heavy and light chains, wherein each heavy chain comprises mutations V173C and C220S and each light chain comprises mutations S162C and C214S.

In some embodiments, the ADC comprises two pairs of heavy and light chains, wherein each heavy chain comprises mutations S134C and C220S and each light chain comprises mutations F116C and C214S.

In some embodiments, the ADC comprises two pairs of heavy and light chains, wherein each heavy chain comprises mutations K133C and C220S and each light chain comprises mutations F209C and C214S.

In some embodiments, the ADC comprises two pairs of heavy and light chains, wherein each heavy chain comprises mutations K133C and C220S and each light chain comprises mutations I117C and C214S.

In some embodiments, the ADC comprises two pairs of heavy and light chains, wherein each heavy chain comprises mutations P130C and C220S and each light chain comprises mutations F118C and C214S.

In some embodiments, the ADC comprises two pairs of heavy and light chains, wherein each heavy chain comprises mutations A141C and C220S and each light chain comprises mutations F116C and C214S.

As used herein, the term “naturally-occurring” or “wild-type” refers to the form found in nature. For example, a naturally occurring or wild-type polypeptide or polynucleotide sequence is a sequence present in an organism which has not been intentionally modified by human manipulation.

When referring to “the first” and “the second” herein, it is used to merely distinguish the two domains or the two chains, but not to indicate the positions of the two domains or two chains in any way.

As used herein, the terms “comprise” or “include” are intended to be inclusive of the stated elements, integers, or steps, but not to exclude any other elements, integers, or steps. When the term “comprise” or “include” is used herein, it also encompasses a combination of the stated elements, integers, or steps, unless otherwise indicated. For example, when referring to an antibody variable region “comprising” a particular sequence, it is also intended to encompass the antibody variable region consisting of that particular sequence.

As used herein, the term “modified” means: a protein or a polypeptide, which has an amino acid sequence comprising a substitution, deletion, addition or insertion of one or more amino acids in the amino acid sequence of the original protein or polypeptide; or a protein or a polypeptide, which has a sequence identity of at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% with all or part of the amino acid sequence of the original protein or polypeptide. The sequence identity of an amino acid sequence can be obtained by a known method of comparing amino acid sequences, and for example, such comparison can be carried out using BLAST (Basic Local Alignment Search Tool at the National Center for Biological Information) or the like at default settings.

The term “amino acid substitution” or “amino acid mutation” refers to the replacement of at least one amino acid residue in a predetermined parent amino acid sequence with a different “substituted” amino acid residue. The substituted residue or residues may be a “naturally occurring amino acid residue” (i.e., encoded by the genetic code) and are selected from the group consisting of: Alanine (Ala); Arginine (Arg); Asparagine (Asn); Aspartic acid (Asp); Cysteine (Cys); Glutamine (Gln); Glutamic acid (Glu); Glycine (Gly); Histidine (His); Isoleucine (Ile); Leucine (Leu); Lysine (Lys); Methionine (Met); Phenylalanine (Phe); Proline (Pro); Serine (Ser); Threonine (Thr); Tryptophan (Trp); Tyrosine (Tyr); and Valine (Val). Substitution with one or more non-naturally occurring amino acid residues is also encompassed within the definition of amino acid substitution herein. “Non-naturally occurring amino acid residues” refers to residues, other than those naturally occurring amino acid residues listed above, that are capable of covalently binding to adjacent amino acid residues in a polypeptide chain. Examples of non-naturally occurring amino acid residues include norleucine, ornithine, norvaline, homoserine, Aib, and other amino acid residue analogs.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the terms “higher than”, “lower than”, “more than”, or “less than” a certain value also encompass a range equal to that value.

“Antibody-drug conjugate” or ADC refers to a conjugate formed by covalently coupling a drug to an antibody directly or indirectly via one or more suitable linkers. ADC is generally in a format of antibody-linker-drug conjugate. The antibody-drug conjugates combine ideal properties of both antibodies and cytotoxic drugs by targeting potent cytotoxic drugs to the antigen-expressing tumor cells, thereby enhancing their anti-tumor activity.

As used herein, the term “drug” as used herein refers to any therapeutic or diagnostic molecule. In some embodiments, the therapeutic molecule is a cytotoxic agent. In some embodiments, the therapeutic molecule has an antitumor effect. In some embodiments, the therapeutic molecule at least one substituted group or a partial structure allowing connection to a linker structure. In some embodiments, the drug may kill cancer cells and/or inhibit growth, proliferation, or metastasis of cancer cells, thereby reducing, alleviating, or eliminating one or more symptoms of a disease or disorder.

As used herein, the term “antibody” as used herein encompasses any immunoglobulin, monoclonal antibody, polyclonal antibody, multispecific antibody, or bispecific (bivalent) antibody that binds to a specific antigen. A native intact antibody comprises two heavy chains and two light chains. Each heavy chain has a variable region and a first, second, and third constant region (CH1, CH2 and CH3), while each light chain has a variable region and a constant region (CL). Mammalian heavy chains are classified as α, δ, ε, γ, and μ, and mammalian light chains are classified as 2 or K. The variable regions generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for antibodies may be defined or identified by the conventions of Kabat, Chothia, or Al-Lazikani (Al-Lazikani, B., Chothia, C., Lesk, A. M., J. Mol. Biol., 273 (4), 927 (1997); Chothia, C. et al., J Mol Biol. December 5; 186 (3): 651-63 (1985); Chothia, C. and Lesk, A. M., J. Mol. Biol., 196,901 (1987); Chothia, C. et al., Nature. December 21-28; 342 (6252): 877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Each variable region comprises four FRs, and the CDRs and FRs are arranged from amino terminus to carboxy terminus in the order: FR1, CDR1, FR2, CDR2, FR3, CDR3, FR4. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain). In the context of antibodies, “a pair of heavy and light chains” means one heavy chain and one light chain that pair up to form an antigen-binding site.

The term “CHI region” refers to the portion of an antibody heavy chain polypeptide extending from EU position 118 to EU position 220 (EU numbering system). In one embodiment, the CH1 region is a CH1 region of human IgG1, IgG2, IgG3 or IgG4.

In one embodiment, the CH1 region comprises the amino acid sequence of ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSC (SEQ ID NO: 90).

The term “Fc domain” or “Fc region” is used herein to define the C-terminal region of an immunoglobulin heavy chain containing at least a portion of the constant region. This term includes native sequence Fc regions and variant Fc regions. A native immunoglobulin “Fc domain” comprises two or three constant domains, namely a CH2 domain, a CH3 domain and an optional CH4 domain. For example, in natural antibodies, the immunoglobulin Fc domain comprises the second and third constant domains (CH2 domain and CH3 domain) derived from the two heavy chains of the IgG, IgA, and IgD antibodies; or comprises the second, third and fourth constant domains (CH2 domain, CH3 domain and CH4 domain) derived from the two heavy chains of IgM and IgE antibodies. Unless otherwise stated herein, amino acid residue number in the Fc region or heavy chain constant region is numbered according to the EU numbering system (also known as the EU Index) as described in Kabat et al., Sequences of Proteins of Immunological Interes, 5th Edition, Public Health Service, National Institutes of Health, Bethesda, MD, 1991. However, the C-terminal lysine (Lys447) of the Fc region may or may not exist. Two Fc regions can dimerize to form a dimeric Fc, and two different Fc regions can heterodimerize to form a heterodimeric Fc. As used herein, the terms “Fc region”, “Fc portion” and “dimeric Fc (e.g., heterodimeric Fc)” do not include the heavy chain variable region VH and the light chain variable region VL as well as the heavy chain constant region CH1 and the light chain constant region CL of an immunoglobulin, but may include a hinge region in the N-terminal of the heavy chain constant region in some cases. In one embodiment, the human IgG heavy chain Fc region extends from Asp221 or from Cys226 or from Asp231 to the carboxy terminus of the heavy chain.

In one embodiment, the Fc region is derived from a human Fc region. The Fc region of the antibody is directly involved in complement activation, Clq binding, C3 activation and Fc receptor binding. In one embodiment, the Fc region is a human Fc region. In one embodiment, the Fc region belongs to the human IgG4 subclass. In one embodiment, the Fc region belongs to the human IgG1 subclass.

Amino acid mutations are represented by (original amino acid, amino acid position, mutated amino acid). When the mutation site is located in the C region, “Q124C” means that glutamine at EU position 124 is substituted by cysteine (C). When referring to combinations of mutations, the mutations in the combination are connected by “/”. “F126C/C220S” means containing both mutations F126C and C220S. The multiple mutation probabilities at a specific position is represented with the symbol “/” herein. For example, the mutation “C220A/V/S” means that residue C at position 220 can be substituted by an A, V, or S residue.

When referring to amino acid positions in the present invention, if not explicitly stated otherwise, it means to the amino acid positions numbered according to the IgG1 heavy chain or kappa light chain, that is, it covers the amino acid positions numbered based on the IgG1 heavy chain or kappa light chain, as well as an amino acid position in other heavy chains or light chain corresponding to said amino acid position. For example, when referring to C220, it encompasses amino acid at position 220 under EU numbering in the IgG1 heavy chain, as well as the corresponding amino acid in other heavy chains, such as amino acid at position 131 in IgG2, IgG3 or IgG4.

It should be noted that when describing mutations, the original amino acid at a specific position may be the amino acid described, or it may be other amino acids at the corresponding position. For example, it should be noted that when describing the mutation S162C, it encompasses mutating the serine at position 162 of CH1 to cysteine, and also encompasses mutating the corresponding amino acid at corresponding position 162 to cysteine when the amino acid at corresponding position 162 is not serine, provided that the original amino acid corresponds to position 162 of the constant region.

As used herein, “antibody fragments” comprise a portion of a full-length antibody, generally the antigen binding or variable region thereof. Examples of antibody fragments include Fab, Fab′, F(ab′)2, and Fv fragments; diabodies; linear antibodies; minibodies (Olafsen et al. (2004) Protein Eng. Design & Sel. 17 (4): 315-323), fragments produced by a Fab expression library, anti-idiotypic (anti-Id) antibodies, CDR (complementary determining region), and epitope-binding fragments of any described herein which immunospecifically bind to cancer cell antigens, viral antigens or microbial antigens, single-chain antibody molecules; and multispecific antibodies formed from antibody fragments. In some embodiments, the antibody fragment is antigen-binding fragment.

As used herein, a “disulfide bond” refers to a covalent bond with the structure R—S—S—R′. The amino acid cysteine comprises a thiol group that can form a disulfide bond with a second thiol group, for example from another cysteine residue. The disulfide bond can be formed between the thiol groups of two cysteine residues residing respectively on the two polypeptide chains, thereby forming an interchain bridge or interchain bond. As used herein, the term “non-native disulfide bond” refers to a disulfide bond that does not naturally exist in a wild-type protein. In some embodiments, the non-native disulfide bond is formed by two cysteine residues, wherein at least one of them is a mutation. In some embodiments, two of them are mutations. In some embodiments, at least one or two cysteine residues are introduced by substitutions. In contrast, a “native disulfide bond” refers to a disulfide bond that naturally exists in a wild-type protein.

As used herein, the term “EU numbering” or “EU numbering system” refers to the EU numbering convention for the constant regions of an antibody, as described in Edelman, G. M. et al., Proc. Natl. Acad. USA, 63, 78-85 (1969) and Kabat et. al, Sequences of Proteins of Immunological Interest, U.S. Dept. Health and Human Services, 5th Edition, 1991, each of which is herein incorporated by reference in its entirety.

As used herein, the term “solvent accessible surface area (SASA)” refers to a value calculated by representing each atom as a collection of grid points distributed over a sphere whose radius is the sum of the atom's van der Waals radius and of a probe radius (1.4 Å). A grid point which is buried inside another atomic sphere contributes to the buried surface area. A grid point which is not buried inside another atomic sphere contributes to the exposed surface area. SASA can be calculated using the Discovery Studio software version 2022.

As used herein, the term “relative solvent accessible surface area (SASA)” in percentage is calculated by the below formula: 100 times the residue SASA divided by the residue SASA of the fully exposed amino acid residue calculated using the extended Ala-X-Ala tripeptide, where X is the residue of interest.

As used herein, “antibody-drug conjugate (ADC)” refers to a compound obtained by linking an antibody to a (small molecule) drug via a linker.

The term “linker” refers to a structural fragment that links a drug (e.g., a small molecule drug) to an antibody moiety. It is understood that the linker, prior to attachment to the antibody or antigen-binding fragment thereof, has a functional group that can form a bond with a functional group of the antibody or antigen-binding fragment thereof.

The term “linker-payload” refers to a compound formed by linking a payload, such as a drug (e.g., a small molecule drug), to a linker.

As used herein, the term “alkyl” refers to a fully saturated branched or unbranched hydrocarbon group. The alkyl group preferably contains 1 to 16 carbon atoms, for example 1 to 12 carbon atoms, 1 to 10 carbon atoms, 1 to 6 carbon atoms or 1 to 4 carbon atoms. Representative examples of alkyl groups include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like.

The term “alkylene” refers to an alkyl group as defined above, but which is divalent, i.e., has two single bonds linked to two other groups. Non-limiting examples of alkylene groups include —CH₂—, —CH₂CH₂—, —CH₂CH₂CH₂—, —CH₂CH₂CH₂CH₂—, —CH(—CH₂CH₃)— or —CH₂CH(—CH₃)—.

The term “alkenyl” refers to a straight or branched chain hydrocarbon group containing 2 to 16 carbon atoms and containing at least one double bond and no triple bond. The alkenyl group preferably contains 2 to 12 carbon atoms, 2 to 10 carbon atoms, 2 to 8 carbon atoms, 2 to 6 carbon atoms, or 2 to 4 carbon atoms. Representative examples of alkenyl groups include, but are not limited to, ethenyl, propenyl, butenyl, pentenyl, hexenyl, and the like.

The term “alkynyl” refers to a straight or branched hydrocarbon group containing 2 to 16 carbon atoms and comprising at least one triple bond. Alkynyl groups preferably contain 2-12 carbon atoms, 2-10 carbon atoms, 2-8 carbon atoms, 2-6 carbon atoms, or 2-4 carbon atoms. Representative examples of alkynyl groups include, but are not limited to, ethynyl, propynyl, butynyl, pentynyl, hexynyl, and the like.

The term “halogen” or “halo” refers to fluoro (—F), chloro (—Cl), bromo (—Br), and iodo (—I).

The term “haloalkyl” refers to an alkyl group, as defined herein, substituted with one or more halo groups as defined herein. The haloalkyl group may preferably be a monohaloalkyl group, a dihaloalkyl group or a polyhaloalkyl group (including a perhaloalkyl group). The monohaloalkyl group may contain one iodo, bromo, chloro or fluoro in the alkyl group. The dihaloalkyl and polyhaloalkyl groups may contain two or more of the same halogen atoms in the alkyl group or a combination of different halo groups. Preferably, the polyhaloalkyl contains up to 12, 10 or 8 or 6 or 4 or 3 or 2 halo groups. Non-limiting examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl, and dichloropropyl. Perhaloalkyl refers to alkyl groups in which all hydrogen atoms are replaced by halogen atoms.

The term “haloalkenyl” refers to an alkenyl group, as defined herein, substituted with one or more halo groups as defined herein. The term “haloalkynyl” refers to an alkynyl group, as defined herein, which is substituted with one or more halo groups as defined herein. The meaning of “halo” as defined for “haloalkyl” is applicable to “haloalkenyl” and “haloalkynyl”.

The term “amino acid” refers to both naturally occurring and synthetic amino acids. The amino acids may be either L or D isomers. The conventional amino acids referred to herein are written following a conventional approach. See, for example, Immunology-A Synthesis (2nd Edition, E. S. Golub and D. R. Gren, eds., Sinauer Associates, Sunderland, Mass. (1991)) which is incorporated herein by reference. And in the present disclosure, amino acids are generally represented by the single and three letter abbreviations commonly known in the art. For example, amino acids may be selected from the group consisting of phenylalanine (Phe; F), tyrosine (Tyr; Y), leucine (Leu; L), glycine (Gly; G), alanine (Ala; A), valine (Val; V), lysine (Lys; K), citrulline (Cit), serine (Ser; S), glutamic acid (Glu; E), aspartic acid (Asp; D), asparagine (Asn), isoleucine (Ile), arginine (Arg), proline (Pro) and glutamine (Gln).

The term “optional” or “optionally” means that the subsequently described event or condition occurs or does not occur, and that the description includes instances where said event or condition occurs and instances where it does not. For example, when a group or structure is “optionally substituted”, the group or structure may or may not be substituted.

The term “pharmaceutically acceptable salt” refers to a salt that retains the biological effects of the ADC conjugates of the invention, and which is not biologically or otherwise undesirable. The ADC conjugates of the invention may exist in the form of their pharmaceutically acceptable salt, including acid addition salts and base addition salts. In the present invention, pharmaceutically acceptable acid addition salts refer to salts formed by the ADC conjugates of the invention with organic or inorganic acids including, but not limited to, hydrochloric acid, sulfuric acid, hydrobromic acid, hydroiodic acid, phosphoric acid, nitric acid, perchloric acid, acetic acid, oxalic acid, maleic acid, fumaric acid, tartaric acid, benzenesulfonic acid, methanesulfonic acid, salicylic acid, succinic acid, citric acid, lactic acid, propionic acid, benzoic acid, p-toluenesulfonic acid, malic acid, and the like. Pharmaceutically acceptable base addition salts mean salts formed by the ADC conjugates of the invention with organic or inorganic bases, including but not limited to alkali metal salts, such as lithium, sodium or potassium salts; alkaline earth metal salts, such as calcium or magnesium salts; organic base salts, for example ammonium salts, formed with organic bases containing N groups.

The term “solvate” refers to an associated complex of one or more solvent molecules with an ADC antibody-drug conjugate of the invention. Solvents that form solvates include, but are not limited to, water, methanol, ethanol, isopropanol, ethyl acetate, tetrahydrofuran, N-dimethylformamide, dimethylsulfoxide, and the like.

“Pharmaceutically acceptable” and “pharmaceutically useful” are used interchangeably herein, unless it conflicts with the context,

The term “drug to antibody ratio” or “DAR” refers to the ratio of drug moiety (D) conjugated to the Ab moiety as described herein to the Ab moiety. In some embodiments described herein, the DAR may be determined by p in formula I. For example, DAR may be from 1 to 16, e.g. 2-16, 4-16, 5-12, 6-10, 2-8, 3-8, 2-6, 4-6, 6-10, e.g. 2, 3, 4, 5, 6, 7, 8, 9, 10, 11, or 12. DAR may also be calculated as the average DAR of a population of molecules in a product, i.e., the overall ratio of drug moiety (D) conjugated to the Ab moiety described herein to the Ab moiety as measured by detection methods (e.g., by conventional methods such as chromatography, mass spectrometry, ELISA assays, electrophoresis, and/or HPLC) in a product, such DAR being referred to herein as average DAR or measured DAR. In some embodiments, the average DAR value of a conjugate of the invention is 1 to 16, e.g., 2-16, 4-16, 5-12, 6-10, 2-8, 3-8, 2-6, 4-6, 6-10, e.g., 1.0-8.0, 2.0-6.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or10.0, and the ranges with two of these values as endpoints. It should be understood that, in the case that an average DAR value is referred to, the ADCs according to the present invention refer to a population of ADC molecules or a mixture of ADC molecules comprising ADC molecules with the same and/or different DARs.

The term “therapeutic agent” as described herein comprises any substance effective in preventing or treating tumors (such as cancer), including a chemotherapeutic agent, a cytokine, angiogenesis inhibitor, a cytotoxic agent, other antibodies, a small molecule drug or an immunomodulatory agent (such as an immunosuppressant).

The term “cytotoxic agent” used in the invention refers to a substance that inhibits or prevents the cell function and/or causes cell death or destruction.

“Chemotherapeutic agents” include chemical compounds useful in treatment of cancer or immune system disease.

The term “prodrug” refers to a chemically modified active or inactive compound that, upon administration to a subject, undergoes a physiological effect in vivo (e.g., hydrolysis, metabolism, etc.) to become the active drug. Techniques for making and using prodrugs are well known to those skilled in the art.

The term “small molecule drugs” refers to drugs with low molecular weight. “Small molecule” is defined as a molecule with molecular weight less than 10 kD, generally less than 2 kD and preferably less than 1 kD, more preferably less than 500 D. Small molecules include but are not limited to organic molecules, organic molecules containing inorganic components, molecules containing radioactive atoms, synthetic molecules, peptide mimics and antibody mimics. As a therapeutic agent, small molecules can penetrate cells more easily than large molecules, and are less susceptible to degradation and less prone to trigger immune response.

“Anti-tumor compounds” are pharmaceutically active compounds that have an effect on a tumor, including but not limited to cytotoxic agents or chemotherapeutic agents, especially small molecule cytotoxic agents or chemotherapeutic agents, including topoisomerase I inhibitors; small molecule Tubulin inhibitors; for example, camptothecins such as those disclosed in WO2021/173773, WO2022180581, CN 102574866, etc., Exatecan (a topoisomerase I inhibitor), Dxd (a Exatecan derivative as a novel topoisomerase I inhibitor); auristatins such as monomethyl auristatin E (MMAE), MMAF; or maytansinoids such as small molecule tubulin inhibitors DM1 and DM4. It should be understood that anti-tumor compounds may be substituted with isotopes including, but not limited to, deuterium, tritium, and the like. For example, following substitution with deuterium, i.e. the carbon-deuterium bond replaces the carbon-hydrogen bond, since the former is more stable than the latter, this substitution can directly affect the certain properties of drugs such as absorption, distribution, metabolism, excretion, etc. thereby improving the efficacy, safety and tolerability of the drugs. Therefore, the “anti-tumor compounds” of the present application may encompass compounds substituted with deuterium.

“Substituted with deuterium” is meant that a hydrogen in the molecule is replaced with deuterium, for example 1 or more hydrogens, for example 1 to 10 (e.g. 1, 2, 3, 4, 5, 6, 7, 8, 9 or 10) hydrogens are replaced with deuterium.

“Camptothecins” refer to compounds that have the core structure of camptothecin (for example, the fused pentacyclic structure of camptothecin or fused tetracyclic structure therein) and have an anti-tumor activity. “Camptothecins” is a term commonly used in medicinal chemistry. Based on the structure of the compound, those skilled in the art can easily determine whether a compound belongs to the Camptothecins.

“Auristatins” refer to compounds that have the core structure of auristatin and have an anti-tumor activity.

“Maytansinoids” refers to compounds that have the core structure of maytansinoid and have an anti-tumor activity.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of the ADC structure.

FIG. 2 shows the RP-HPLC analysis results.

FIG. 3 shows the SEC analysis results.

FIG. 4 shows the HIC analysis results.

FIG. 5 is a schematic illustration of the ADC structure.

FIG. 6 shows the RP-HPLC analysis results.

FIG. 7 shows the SEC analysis results.

FIG. 8 shows the HIC analysis results.

FIG. 9 is a schematic illustration of the ADC structure.

FIG. 10 shows the RP-HPLC analysis results.

FIG. 11 shows the SEC analysis results.

FIG. 12 shows the HIC analysis results.

FIG. 13 is a schematic illustration of the ADC structure.

FIG. 14 shows the RP-HPLC analysis results.

FIG. 15 shows the SEC analysis results.

FIG. 16 shows the HIC analysis results.

FIG. 17 is a schematic illustration of the ADC structure.

FIG. 18 shows the RP-HPLC analysis results.

FIG. 19 shows the SEC analysis results.

FIG. 20 shows the HIC analysis results.

FIG. 21 is a schematic illustration of the ADC structure.

FIG. 22 shows the RP-HPLC analysis results.

FIG. 23 shows the SEC analysis results.

FIG. 24 shows the HIC analysis results.

FIG. 25 is a schematic illustration of the ADC structure.

FIG. 26 shows the RP-HPLC analysis results.

FIG. 27 shows the HIC analysis results.

FIG. 28 is a schematic illustration of the ADC structure.

FIG. 29 shows the RP-HPLC analysis results.

FIG. 30 shows the SEC analysis results.

FIG. 31 shows the HIC analysis results.

FIG. 32 is a schematic illustration of the ADC structure.

FIG. 33 shows the RP-HPLC analysis results.

FIG. 34 shows the SEC analysis results.

FIG. 35 is a schematic illustration of the ADC structure.

FIG. 36 shows the RP-HPLC analysis results.

FIG. 37 shows the SEC analysis results.

FIG. 38 shows the RP-HPLC analysis results.

FIG. 39 shows the RP-HPLC analysis results.

FIG. 40 shows the RP-HPLC analysis results.

FIG. 41 shows the RP-HPLC analysis results.

FIG. 42 shows the RP-HPLC analysis results.

FIG. 43 shows the RP-HPLC analysis results.

FIG. 44 shows the RP-HPLC analysis results.

FIG. 45 shows the RP-HPLC analysis results.

FIG. 46 shows the RP-HPLC analysis results.

FIG. 47 shows the RP-HPLC analysis results.

FIG. 48 shows the RP-HPLC analysis results.

FIG. 49 shows the RP-HPLC analysis results.

FIG. 50 shows the HIC analysis results.

FIG. 51 shows the RP-HPLC analysis results.

FIG. 52 shows the HIC analysis results.

FIG. 53 shows the RP-HPLC analysis results.

FIG. 54 shows the HIC analysis results.

FIG. 55 shows the RP-HPLC analysis results.

FIG. 56 shows the HIC analysis results.

FIG. 57 shows the RP-HPLC analysis results.

FIG. 58 shows the HIC analysis results.

FIG. 59 is a schematic illustration of the ADC structure.

FIG. 60 shows the RP-HPLC analysis results.

FIG. 61 shows the HIC analysis results.

FIG. 62 is a schematic illustration of the ADC structure.

FIG. 63 shows the RP-HPLC analysis results.

FIG. 64 shows the HIC analysis results.

FIG. 65 is a schematic illustration of the ADC structure.

FIG. 66 shows the RP-HPLC analysis results.

FIG. 67 shows the HIC analysis results.

FIG. 68 is a schematic illustration of the ADC structure.

FIG. 69 shows the RP-HPLC analysis results.

FIG. 70 shows the HIC analysis results.

FIG. 71 is a schematic illustration of the ADC structure.

FIG. 72 shows the RP-HPLC analysis results.

FIG. 73 shows the HIC analysis results.

FIG. 74 is a schematic illustration of the ADC structure.

FIG. 75 shows the linear relationship between DAR and TCEP equivalent.

FIG. 76 shows the HIC analysis results.

FIG. 77 shows the RP-HPLC analysis results.

FIG. 78 shows the SEC analysis results.

FIG. 79 is a schematic illustration of the ADC structure.

FIG. 80 shows the linear relationship between DAR and TCEP equivalent.

FIG. 81 shows the RP-HPLC analysis results.

FIG. 82 shows the SEC analysis results.

FIG. 83 shows the HIC analysis results.

FIGS. 84-90 show the plasma stability results.

FIG. 91 shows the cell binding assay results. The X-axis is the concentration of the antibody or ADC. The Y-axis is the fluorescence intensity.

FIG. 92 shows the cytotoxicity assay results. The X-axis is the concentration of the antibody or ADC. The Y-axis is cell viability.

FIG. 93 shows the average tumor volumes in different groups of CB17-SCID mice that were injected with NCI-N87 cells, and were treated with ADCs.

FIG. 94 shows the average body weight changes in different groups of CB17-SCID mice that were injected with NCI-N87 cells, and were treated with ADCs.

FIG. 95 is a schematic illustration of the ADC structure.

FIG. 96 shows the RP-HPLC analysis results.

FIG. 97 shows the HIC analysis results.

FIG. 98 is a schematic illustration of the ADC structure.

FIG. 99 shows the RP-HPLC analysis results.

FIG. 100 shows the HIC analysis results.

FIG. 101 lists relevant amino acid sequences.

### DETAILED DESCRIPTION

Antibody-drug conjugates (ADCs) have emerged as an important class of biologics for targeted treatment of cancers. So far 14 ADCs have been approved by the US FDA. ADCs are complex molecules composed of an antibody linked to biologically active cytotoxic drugs. Conventional strategies for drug attachment (i.e. lysine- and interchain cysteine-based conjugation to achieve an average drug-to-antibody ratio (DAR) of target level) suffer from heterogeneity of conjugation sites and attached drugs. Progress in the development of site-specific conjugation including Thiomab, disulfide rebridging, incorporation of non-natural amino acids and enzymatic bioconjugation, has enabled the preparation of homogeneous ADCs. However, most of the strategies require re-engineering of the antibody sequence, multistep operation and costly substrates, thus limit the scope of application in industry.

Encouraged by the clinical success of Enhertu and Trodelvy, interchain cysteine-based conjugation has become a dominant strategy for the development of novel ADCs. Apart from DAR8 ADCs that open up all the interchain disulfide bonds for conjugation, tunable DARs, i.e. DAR4 and DAR6 are extremely desirable if highly potent drugs or dual drugs with different mechanisms are used as the payloads. One strategy is replacing part of the interchain cysteines with serine, thus reducing the eight potential conjugation sites down to 4, or 6. Although homogeneous DAR6 and DAR4 ADCs can be successfully generated, the removal of interchain disulfide bonds may destabilize the antibody and cause the dissociation of the light chain. The present disclosure provides a cost-efficient, easy-to-operate cysteine-based conjugation method that provides site-specific ADCs with stable structures and defined DARs.

In one aspect, the present disclosure combined in silico analysis and experimental testing and finally defined certain engineered cysteine pairs for the synthesis of stable and homogeneous DAR4 and DAR6 ADCs in a cost-efficient and practical way. In some embodiments, the engineered cysteine pairs could form stable disulfide bonds that are resistant to TCEP reduction. In some embodiments, the resulting DAR4 or DAR6 species accounted for ~90% of the products.

In some embodiments, the present disclosure provides modified antibodies and antibody fragments that can be used in the synthesis of site-specific DAR4 and DAR6 monospecific ADC, DAR4 and DAR6 bispecific ADC. In some embodiments, the modified antibodies and antibody fragments can be used to produce more homogenous ADC products. In some embodiments, the payload can be selected from cytotoxic drugs, biologically active proteins, synthetic polymers, enzymes, nucleic acids and fragments thereof e.g. DNA, or RNA.

### Modified Antibodies and Antibody Fragments Thereof and Thiol-Based Conjugation

To avoid side effects, drugs should be specifically delivered to cancer cells via binding to ligands that can specifically recognize the cancer-associated biomarkers such as antigens. Among the ligands for targeted therapy, antibodies are excellent candidates because of their specific recognitions and high affinities. Nowadays, antibody-drug conjugates (ADCs) are attracting tremendous attention for targeted cancer therapy.

Antibody-drug conjugates are biotherapeutics that comprise antibodies, potent drugs and linkers between them. The antibodies lead the drugs to the target cancer cells. In some embodiments, the drug is a prodrug, which can be chemically or enzymatically converted to their active forms. Conjugating cytotoxins to antibodies that specifically tie to tumor cell surface antigens enables the drugs to be target-delivered to cancer cells and leaves normal cells unaffected. More important, many of the cytotoxic drugs that are too toxic for use in traditional chemotherapy can also be used in the construction of antibody-drug conjugates. The linkers are also essential parts of antibody-drug conjugates, which account for stability in circulation, good pharmacokinetics and efficient release of toxic drugs in the tumor cells.

Employing the thiols of interchain cysteine residues in monoclonal antibodies as attachment sites for drug molecules is one of the most used conjugation methods. In a human IgG1, there are four interchain disulfide bonds that can be used as potential conjugation sites. The four interchain disulfide bonds can be reduced by tris(2-carboxyethyl)phosphine (TCEP) or dithiothreitol (DTT), which results in eight thiol groups that are available for conjugating drug molecules. Through this method, different drug antibody ratio (DAR) conjugates will be obtained when targeting typical DARs of 2-6. In addition, antibody-drug conjugate at each drug antibody ratio has several isomers. Thus, many different species are present in the antibody-drug conjugate.

Homogeneous antibody-drug conjugates can be produced through cysteine residues when all interchain cysteines are coupled to drugs. An exemplary ADC contains cAC10, an anti-CD30 monoclonal antibody, and monomethyl auristatin E (MMAE). This cAC10-vcMMAE conjugate contains eight drugs per antibody, which is the highest drug antibody ratio (DAR) that can be obtained through using interchain cysteines as conjugation sites. However, antibody-drug conjugates with four drugs per antibody generally have improved in vivo performance.

A detailed review of ADCs and thiol-based conjugation can be found in Yao et al. “Methods to design and synthesize antibody-drug conjugates (ADCs).” International journal of molecular sciences 17.2 (2016): 194, which is incorporated by reference in its entirety.

In one aspect, the present disclosure provides modified antibodies and antibody fragments thereof where one or more of the natural heavy-light chain (interchain) disulfide bonds are relocated to a solvent inaccessible region so that these disulfide bonds will not be opened by the typical reducing conditions. As a result, the maleimide-derived linker-payloads can be site-specifically conjugated at the other interchain cysteine residues to achieve defined DARs. In some embodiments, both or one of the disulfide bonds between the heavy and light chain (heavy-light chain disulfide bonds) were mutated and the linker-payload was conjugated at the hinge region to provide DAR4 and DAR6 ADCs, respectively.

In some embodiments, the amino acid pairs on the binding interface of the heavy and light chains are screened, and a panel of engineered cysteine pairs that are solvent inaccessible and prone to form disulfide bond are identified. This analysis has enabled by calculating the solvent accessible surface area (SASA) of the side chains for candidate amino acid pairs in a structure of a Fab fragment, and the formation of disulfide bond is confirmed by non-reducing SDS-PAGE. Site-specific conjugation can be tested with different engineered pairs, different antibodies and different linker-payloads, and the in vitro and in vivo efficacy of the resulting ADCs can be validated in cell assays and animal models, respectively.

In one aspect, the disclosure relates to various modified antibodies and antibody fragments thereof. In some embodiments, various modifications are added to the antibody fragment to modify the DAR of the ADC. In some embodiments, one or more of the Fab region disulfide bonds are moved to a solvent inaccessible region.

In some embodiments, one or more native heavy-light chain disulfide bonds between the heavy chain and light chain is absent in the modified antibody or antibody fragment thereof.

In some embodiments, the native heavy chain-light chain disulfide bonds in the modified antibody or antibody fragment thereof are mutated into non-disulfide bonds.

In some embodiments, the heavy chain cysteine C220 (IgG1 subtype, or comprising C131S/A/V at the corresponding position of IgG2, IgG3 or IgG4) is mutated into a non-cysteine residue. In some embodiments, the heavy chain cysteine C220 is mutated into A, R, N, D, Q, E, G, H, I, L, K, M, F, P, S, T, W, Y, or V, e.g., S, A or V. In some embodiments, the modified antibody or antibody fragment thereof contains the C220S mutation in a heavy chain CH1 region. In some embodiments, the modified antibody or antibody fragment thereof contains two heavy chains and the heavy chain cysteine C220 is mutated into a non-cysteine residue in both heavy chains. In some embodiments, the modified antibody or antibody fragment thereof contains two heavy chains and the heavy chain cysteine C220 is mutated into a non-cysteine residue in only one of the two heavy chains. In some embodiments, the modified antibody or antibody fragment thereof contains two heavy chains, each of which contains the C220S mutation. In some embodiments, the modified antibody or antibody fragment thereof contains two heavy chains and only one heavy chain contains the C220S mutation.

In some embodiments, the light chain cysteine C214 is mutated into a non-cysteine residue. In some embodiments, the light chain cysteine C214 is mutated into A, R, N, D, Q, E, G, H, I, L, K, M, F, P, S, T, W, Y, or V, e.g., S, A or V. In some embodiments, the modified antibody or antibody fragment thereof contains the C214S mutation in a light chain constant region CL. In some embodiments, the modified antibody or antibody fragment thereof contains two light chains and the light chain cysteine C214 is mutated into a non-cysteine residue in both light chains. In some embodiments, the modified antibody or antibody fragment thereof contains two light chains and the light chain cysteine C214 is mutated into a non-cysteine residue in only one of the two light chains. In some embodiments, the modified antibody or antibody fragment thereof contains two light chains, each of which contains the C214S mutation. In some embodiments, the modified antibody or antibody fragment thereof contains two light chains and only one light chain contains the C214S mutation.

In some embodiments, the modified antibody or antibody fragment thereof has a non-native disulfide bond. In some embodiments, the modified antibody or antibody fragment thereof described herein have a non-native disulfide bond at one of the locations in Table 1. In some embodiments, the non-native disulfide bond is located at one of the below CH1/CL locations: F126/Q124, H168/T164, F170/T164, F170/S162, V173/S162, S134/F116, K133/F209, K133/I117, P130/F118, and A141/F116. In some embodiments, the modified antibody or antibody fragment thereof comprises at least a non-native disulfide bond in a solvent inaccessible region. In some embodiments, the modified antibody or antibody fragment thereof contains two pairs of heavy and light chains, each pair of which contain one of the following CH1/CL mutations F126/Q124, H168/T164, F170/T164, F170/S162, V173/S162, S134/F116, K133/F209, K133/I117, P130/F118, and A141/F116. In some embodiments, the modified antibody or antibody fragment thereof contains two pairs of heavy and light chains, and only one pair of heavy and light chains contain one of the following CH1/CL mutations: F126/Q124, H168/T164, F170/T164, F170/S162, V173/S162, S134/F116, K133/F209, K133/I117, P130/F118, and A141/F116.

In some embodiments, one amino acid in the heavy chain constant region of the modified antibody or antibody fragment thereof is mutated to a cysteine. In some embodiments, the amino acid is selected from the group consisting of: F126, H168, F170, V173, S134, K133, P130, and A141. In some embodiments, the modified antibody or antibody fragment thereof contains one or more of the following mutations in the heavy chain constant region: F126C, H168C, F170C, V173C, S134C, K133C, P130C, and A141C. In some embodiments, the modified antibody or antibody fragment thereof contains two heavy chains, each of which contains one or more of the following mutations in the heavy chain constant region F126C, H168C, F170C, V173C, S134C, K133C, P130C, and A141C. In some embodiments, the modified antibody or antibody fragment thereof contains two heavy chains, and only one heavy chain contains one or more of the following mutations in the heavy chain constant region: F126C, H168C, F170C, V173C, S134C, K133C, P130C, and A141C.

In some embodiments, one amino acid in the light chain constant region of the modified antibody or antibody fragment thereof is mutated to a cysteine. In some embodiments, the amino acid is selected from the group consisting of: Q124, T164, S162, F116, F209, 1117, and F118. In some embodiments, the modified antibody or antibody fragment thereof contains one or more of the following mutations in the light chain constant region: Q124C, T164C, S162C, F116C, F209C, I117C, and F118C. In some embodiments, the modified antibody or antibody fragment thereof contains two light chains, each of which contains one or more of the following mutations in the light chain constant region: Q124C, T164C, S162C, F116C, F209C, I117C, and F118C. In some embodiments, the modified antibody or antibody fragment thereof contains two light chains, and only one light chain contains one or more of the following mutations in the light chain constant region: Q124C, T164C, S162C, F116C, F209C, I117C, and F118C.

In some embodiments, the modified antibody or antibody fragment thereof has two heavy chain constant region mutations and two light chain constant region mutations. In some embodiments, the modified antibody or antibody fragment thereof has the F126C mutation and the C220S mutation in the heavy chain constant region. In some embodiments, the modified antibody or antibody fragment thereof has the Q124C mutation and the C214S mutation in the light chain constant region. In some embodiments, the modified antibody or antibody fragment thereof has a non-native disulfide bond at the below CH1/CL location: F126/Q124. In some embodiments, the modified antibody or antibody fragment thereof has two heavy chains, each of which has the F126C mutation and the C220S mutation in the heavy chain constant region. In some embodiments, the modified antibody or antibody fragment thereof has two light chains, each of which has the Q124C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the H168C mutation and the C220S mutation in the heavy chain constant region. In some embodiments, the modified antibody or antibody fragment thereof has the T164C mutation and the C214S mutation in the light chain constant region. In some embodiments, the modified antibody or antibody fragment thereof has a non-native disulfide bond at the below CH1/CL location: H168/T164. In some embodiments, the modified antibody or antibody fragment thereof has two heavy chains, each of which has the H168C mutation and the C220S mutation in the heavy chain constant region. In some embodiments, the modified antibody or antibody fragment thereof has two light chains, each of which has the T164C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the F170C mutation and the C220S mutation in the heavy chain constant region. In some embodiments, the modified antibody or antibody fragment thereof has the T164C mutation and the C214S mutation in the light chain constant region. In some embodiments, the modified antibody or antibody fragment thereof has a non-native disulfide bond at the below CH1/CL location: F170/T164. In some embodiments, the modified antibody or antibody fragment thereof has two heavy chains, each of which has the F170C mutation and the C220S mutation in the heavy chain constant region. In some embodiments, the modified antibody or antibody fragment thereof has two light chains, each of which has the T164C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the F170C mutation and the C220S mutation in the heavy chain constant region. In some embodiments, the modified antibody or antibody fragment thereof has the S162C mutation and the C214S mutation in the light chain constant region. In some embodiments, the modified antibody or antibody fragment thereof has a non-native disulfide bond at the below CH1/CL location: F170/S162. In some embodiments, the modified antibody or antibody fragment thereof has two heavy chains, each of which has the F170C mutation and the C220S mutation in the heavy chain constant region. In some embodiments, the modified antibody or antibody fragment thereof has two light chains, each of which has the S162C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the V173C mutation and the C220S mutation in the heavy chain constant region. In some embodiments, the modified antibody or antibody fragment thereof has the S162C mutation and the C214S mutation in the light chain constant region. In some embodiments, the modified antibody or antibody fragment thereof has a non-native disulfide bond at the below CH1/CL location: V173/S162. In some embodiments, the modified antibody or antibody fragment thereof has two heavy chains, each of which has the V173C mutation and the C220S mutation in the heavy chain constant region. In some embodiments, the modified antibody or antibody fragment thereof has two light chains, each of which has the S162C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the S134C mutation and the C220S mutation in the heavy chain constant region. In some embodiments, the modified antibody or antibody fragment thereof has the F116C mutation and the C214S mutation in the light chain constant region. In some embodiments, the modified antibody or antibody fragment thereof has a non-native disulfide bond at the below CH1/CL location: S134/F116. In some embodiments, the modified antibody or antibody fragment thereof has two heavy chains, each of which has the S134C mutation and the C220S mutation in the heavy chain constant region. In some embodiments, the modified antibody or antibody fragment thereof has two light chains, each of which has the F116C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the K133C mutation and the C220S mutation in the heavy chain constant region. In some embodiments, the modified antibody or antibody fragment thereof has the F209C mutation and the C214S mutation in the light chain constant region. In some embodiments, the modified antibody or antibody fragment thereof has a non-native disulfide bond at the below CH1/CL location: K133/F209. In some embodiments, the modified antibody or antibody fragment thereof has two heavy chains, each of which has the K133C mutation and the C220S mutation in the heavy chain constant region. In some embodiments, the modified antibody or antibody fragment thereof has two light chains, each of which has the F209C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the K133C mutation and the C220S mutation in the heavy chain constant region. In some embodiments, the modified antibody or antibody fragment thereof has the I117C mutation and the C214S mutation in the light chain constant region. In some embodiments, the modified antibody or antibody fragment thereof has a non-native disulfide bond at the below CH1/CL location: K133/I117. In some embodiments, the modified antibody or antibody fragment thereof has two heavy chains, each of which has the K133C mutation and the C220S mutation in the heavy chain constant region. In some embodiments, the modified antibody or antibody fragment thereof has two light chains, each of which has the I117C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the P130C mutation and the C220S mutation in the heavy chain constant region. In some embodiments, the modified antibody or antibody fragment thereof has the F118C mutation and the C214S mutation in the light chain constant region. In some embodiments, the modified antibody or antibody fragment thereof has a non-native disulfide bond at the below CH1/CL location: P130/F118. In some embodiments, the modified antibody or antibody fragment thereof has two heavy chains, each of which has the P130C mutation and the C220S mutation in the heavy chain constant region. In some embodiments, the modified antibody or antibody fragment thereof has two light chains, each of which has the F118C mutation and the C214S mutation in the light chain constant region.

In some embodiments, the modified antibody or antibody fragment thereof has two heavy chain constant region mutations and two light chain constant region mutations. In some embodiments, the modified antibody or antibody fragment thereof has the A141C mutation and the C220S mutation in the heavy chain constant region. In some embodiments, the modified antibody or antibody fragment thereof has the F116C mutation and the C214S mutation in the light chain constant region. In some embodiments, the modified antibody or antibody fragment thereof has a non-native disulfide bond at the below CH1/CL location: A141/F116. In some embodiments, the modified antibody or antibody fragment thereof has two heavy chains, each of which has the A141C mutation and the C220S mutation in the heavy chain constant region. In some embodiments, the modified antibody or antibody fragment thereof has two light chains, each of which has the F116C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the F126C mutation and the C220S mutation in the heavy chain constant region in a first heavy chain. In some embodiments, the modified antibody or antibody fragment thereof has the Q124C mutation and the C214S mutation in the light chain constant region in a first light chain. In some embodiments, the modified antibody or antibody fragment thereof has a non-native disulfide bond at the below CH1/CL location: F126/Q124. In some embodiments, the modified antibody or antibody fragment thereof has two heavy chains, and only one of the two heavy chains contain the F126C mutation and the C220S mutation in the heavy chain constant region. In some embodiments, the modified antibody or antibody fragment thereof has two light chains, and only one of the two light chains contain the Q124C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the K133C mutation and the C220S mutation in the heavy chain constant region in a first heavy chain. In some embodiments, the modified antibody or antibody fragment thereof has the F209C mutation and the C214S mutation in the light chain constant region in a first light chain. In some embodiments, the modified antibody or antibody fragment thereof has a non-native disulfide bond at the below CH1/CL location: K133/F209. In some embodiments, the modified antibody or antibody fragment thereof has two heavy chains, and only one of the two heavy chains contain the K133C mutation and the C220S mutation in the heavy chain constant region. In some embodiments, the modified antibody or antibody fragment thereof has two light chains, and only one of the two light chains contain the F209C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the K133C mutation and the C220S mutation in the heavy chain constant region in a first heavy chain. In some embodiments, the modified antibody or antibody fragment thereof has the I117C mutation and the C214S mutation in the light chain constant region in a first light chain. In some embodiments, the modified antibody or antibody fragment thereof has a non-native disulfide bond at the below CH1/CL location: K133/I117. In some embodiments, the modified antibody or antibody fragment thereof has two heavy chains, and only one of the two heavy chains contain the K133C mutation and the C220S mutation in the heavy chain constant region. In some embodiments, the modified antibody or antibody fragment thereof has two light chains, and only one of the two light chains contain the I117C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the P130C mutation and the C220S mutation in the heavy chain constant region in a first heavy chain. In some embodiments, the modified antibody or antibody fragment thereof has the F118C mutation and the C214S mutation in the light chain constant region in a first light chain. In some embodiments, the modified antibody or antibody fragment thereof has a non-native disulfide bond at the below CH1/CL location: P130/F118. In some embodiments, the modified antibody or antibody fragment thereof has two heavy chains, and only one of the two heavy chains contain the P130C mutation and the C220S mutation in the heavy chain constant region. In some embodiments, the modified antibody or antibody fragment thereof has two light chains, and only one of the two light chains contain the F118C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the A141C mutation and the C220S mutation in the heavy chain constant region in a first heavy chain. In some embodiments, the modified antibody or antibody fragment thereof has the F116C mutation and the C214S mutation in the light chain constant region in a first light chain. In some embodiments, the modified antibody or antibody fragment thereof has a non-native disulfide bond at the below CH1/CL location: A141/F116. In some embodiments, the modified antibody or antibody fragment thereof has two heavy chains, and only one of the two heavy chains contain the A141C mutation and the C220S mutation in the heavy chain constant region. In some embodiments, the modified antibody or antibody fragment thereof has two light chains, and only one of the two light chains contain the F116C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the H168C mutation and the C220S mutation in the heavy chain constant region in a first heavy chain. In some embodiments, the modified antibody or antibody fragment thereof has the T164C mutation and the C214S mutation in the light chain constant region in a first light chain. In some embodiments, the modified antibody or antibody fragment thereof has a non-native disulfide bond at the below CH1/CL location: H168/T164. In some embodiments, the modified antibody or antibody fragment thereof has two heavy chains, and only one of the two heavy chains contain the H168C mutation and the C220S mutation in the heavy chain constant region. In some embodiments, the modified antibody or antibody fragment thereof has two light chains, and only one of the two light chains contain the T164C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the F170C mutation and the C220S mutation in the heavy chain constant region in a first heavy chain. In some embodiments, the modified antibody or antibody fragment thereof has the T164C mutation and the C214S mutation in the light chain constant region in a first light chain. In some embodiments, the modified antibody or antibody fragment thereof has a non-native disulfide bond at the below CH1/CL location: F170/T164. In some embodiments, the modified antibody or antibody fragment thereof has two heavy chains, and only one of the two heavy chains contain the F170C mutation and the C220S mutation in the heavy chain constant region. In some embodiments, the modified antibody or antibody fragment thereof has two light chains, and only one of the two light chains contain the T164C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the F170C mutation and the C220S mutation in the heavy chain constant region in a first heavy chain. In some embodiments, the modified antibody or antibody fragment thereof has the S162C mutation and the C214S mutation in the light chain constant region in a first light chain. In some embodiments, the modified antibody or antibody fragment thereof has a non-native disulfide bond at the below CH1/CL location: F170/S162. In some embodiments, the modified antibody or antibody fragment thereof has two heavy chains, and only one of the two heavy chains contain the F170C mutation and the C220S mutation in the heavy chain constant region. In some embodiments, the modified antibody or antibody fragment thereof has two light chains, and only one of the two light chains contain the S162C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the V173C mutation and the C220S mutation in the heavy chain constant region in a first heavy chain. In some embodiments, the modified antibody or antibody fragment thereof has the S162C mutation and the C214S mutation in the light chain constant region in a first light chain. In some embodiments, the modified antibody or antibody fragment thereof has a non-native disulfide bond at the below CH1/CL location: V173/S162. In some embodiments, the modified antibody or antibody fragment thereof has two heavy chains, and only one of the two heavy chains contain the V173C mutation and the C220S mutation in the heavy chain constant region. In some embodiments, the modified antibody or antibody fragment thereof has two light chains, and only one of the two light chains contain the S162C mutation and the C214S mutation in the light chain constant region.

In some embodiments, the modified antibody or antibody fragment thereof has two heavy chain constant region mutations in a first heavy chain and two light chain constant region mutations in a first light chain. In some embodiments, the modified antibody or antibody fragment thereof has the S134C mutation and the C220S mutation in the heavy chain constant region in a first heavy chain. In some embodiments, the modified antibody or antibody fragment thereof has the F116C mutation and the C214S mutation in the light chain constant region in a first light chain. In some embodiments, the modified antibody or antibody fragment thereof has a non-native disulfide bond at the below CH1/CL location: S134/F116. In some embodiments, the modified antibody or antibody fragment thereof has two heavy chains, and only one of the two heavy chains contain the S134C mutation and the C220S mutation in the heavy chain constant region. In some embodiments, the modified antibody or antibody fragment thereof has two light chains, and only one of the two light chains contain the F116C mutation and the C214S mutation in the light chain constant region.

Accordingly, in one embodiment of the present disclosure, the present disclosure relates to an antibody comprising a modified CH1/CL, wherein
(i) the CH1 contains the F126C mutation, and the CL contains the Q124C mutation (EU numbering);
(ii) the CH1 contains the H168C mutation, and the CL contains the T164C mutation (EU numbering);
(iii) the CH1 contains the F170C mutation, and the CL contains the T164C mutation (EU numbering);
(iv) the CH1 contains the F170C mutation, and the CL contains the S162C mutation (EU numbering);
(v) the CH1 contains the V173C mutation, and the CL contains the S162C mutation (EU numbering);
(vi) the CH1 contains the S134C mutation, and the CL contains the F116C mutation (EU numbering);
(vii) the CH1 contains the K133C mutation, and the CL contains the F209C mutation (EU numbering);
(viii) the CH1 contains the K133C mutation, and the CL contains the I117C mutation (EU numbering);
(ix) the CH1 contains the P130C mutation, and the CL contains the F118C mutation (EU numbering);
(x) the CH1 contains the A141C mutation, and the CL contains the F116C mutation (EU numbering);
and optionally the CH1 further contains the C220S/A/V mutation (or the corresponding C131S/A/V), and the CL further contains the C214S/A/V mutation (EU numbering);
preferably, the CH1 further contains C220S (EU numbering), and the CL further contains C214S (EU numbering).

In some embodiments, the antibody or antigen-binding fragment thereof comprises a pair of modified CH1/CL, wherein CH1/CL is located on the heavy chain and light chain, respectively. In some embodiments, the antibody or antigen-binding fragment thereof comprises two pairs of modified CH1/CL, wherein CH1/CL is located on the heavy chain and light chain respectively.

In some embodiments, the antibody or antigen-binding fragment thereof comprises two heavy chains and two light chains, wherein the two heavy chains can be the same or different, and the two light chains can be the same or different, e.g., wherein the heavy chain and the light chain contain the following combinations of mutations:

| Combinations | First | First | Second | Second |
|---|---|---|---|---|
| of mutation | heavy chain | light chain | heavy chain | light chain |
| i | F126C/C220S | Q124C/C214S | F126C/C220S | Q124C/C214S |
| ii | H168C/C220S | T164C/C214S | H168C/C220S | T164C/C214S |
| iii | F170C/C220S | T164C/C214S | F170C/C220S | T164C/C214S |
| iv | F170C/C220S | S162C/C214S | F170C/C220S | S162C/C214S |
| v | V173C/C220S | S162C/C214S | V173C/C220S | S162C/C214S |
| vi | S134C/C220S | F116C/C214S | S134C/C220S | F116C/C214S |
| vii | F126C/C220S | Q124C/C214S | / | / |
| viii | S134C/C220S | T116C/C214S | S134C/C220S | T116C/C214S |
| ix | K133C/C220S | I117C/C214S | K133C/C220S | I117C/C214S |
| x | K133C/C220S | F209C/C214S | K133C/C220S | F209C/C214S |
| xi | A141C/C220S | F116C/C214S | A141C/C220S | F116C/C214S |
| xii | P130C/C220 | F118C/C214S | P130C/C220 | F118C/C214S |
| xiii | F170C/C220S | T164C/C214S | / | / |
| xiv | F170C/C220S | S162C/C214S | / | / |
| xv | S134C/C220S | F116C/C214S | / | / |
| xvi | P130C/C220 | F118C/C214S | / | / |

In some embodiments, when the antibody or antibody fragment thereof comprises the Lambda light chain constant region, it may contain the S134C mutation (numbered according to heavy chain IgG1) in the heavy chain constant region, and T116C mutation in the Lambda light chain constant region (numbered according to light chain Lambda).

In some embodiments, when the antibody or antibody fragment thereof comprises two heavy chains and two light chains, wherein both of the light chains comprise a Lambda light chain constant region, the two heavy chains each contains the S134C mutation (numbered according to the heavy chain IgG1) in the heavy chain constant region or CH1, and the two light chains each contains the T116C mutation (numbered according to the light chain Lambda) in the Lambda light chain constant region

In some embodiments, when the antibody or antibody fragment thereof comprises two heavy chains and two light chains, wherein one or both of the light chains comprise a Lambda light chain constant region, one of the heavy chains contains the S134C mutation (numbered according to the heavy chain IgG1) in the heavy chain constant region or CH1, and the light chain paired with the heavy chain containing the Lambda constant region contains the T116C mutation (numbered according to the light chain Lambda) in the Lambda light chain constant region.

In some embodiments, the modified antibody or antibody fragment thereof has one or more additional amino acid mutations. In some embodiments, the modified antibody or antibody fragment thereof can have 1, 2, 3, 4, 5, 6, 7, 8, 9, 10 or more than 10 mutations. In some embodiments, the mutation can be a deletion, an insertion, or a substitution.

In some embodiments, the modified antibody or antibody fragment thereof has a Fc region. In some embodiments, the Fc region is from human IgG1, human IgG2, human IgG3, or human IgG4. In some embodiments, the Fc region comprises or consists of an amino acid sequence having at least 90% identical (e.g., 95%, 96%, 97%, 98%, 99% or higher) to the amino acid sequence SEQ ID NO: 91 or 92.

In one embodiment, the Fc region is modified in characteristics of an effector function of the Fc region (e.g., complement activation function of the Fc region). In one embodiment, the effector function has been reduced or eliminated relative to a wild-type isotype Fc region. In one embodiment, effector function is reduced or eliminated by a method selected from the group consisting of: use of a Fc isoform which naturally has reduced or eliminated effector function, and Fc region modification.

In a preferred embodiment, the Fc region has reduced effector function mediated by the Fc region, such as reduced or eliminated ADCC or ADCP or CDC effector function, e.g. comprising mutations to achieve the above function.

As will be understood by those skilled in the art, the antibody or antibody fragment of the disclosure may also comprise modifications in the Fc domain that alter binding affinity to one or more Fc receptors, depending on the intended use of the antibody or antibody fragment, of the disclosure. In one embodiment, the Fc receptor is an Fc gamma receptor, particularly a human Fc gamma receptor. In some embodiments, the Fc region contains a mutation that reduces binding to an Fc gamma receptor. In yet another preferred embodiment, the Fc region can have a mutation that results in increased serum half-life, such as a mutation that improves binding of the Fc region to FcRn.

In some embodiments, the antibody is a human IgG1 antibody, optionally with SI mutations, LALA mutations, N297A mutation, YTE mutations, and/or FLAA mutations in Fc region. In some embodiments, the antibody is a human IgG4 antibody, optionally with SI mutations, LALA mutations, N297A mutation, YTE mutations, and/or FLAA mutations. In some embodiments, the Fc region has LALA mutations (L234A and L235A mutations according to EU numbering), or LALA-PG mutations (L234A, L235A, P329G mutations according to EU numbering). In some embodiments, the Fc region has FLAA mutations (F234A and L235A according to EU numbering). In some embodiments, the Fc has SI mutations (S239D and I332E mutations according to EU numbering). In some embodiments, the Fc has N297A mutation according to EU numbering. In some embodiments, the Fc has YTE mutations (M252Y, S254T and T256E according to EU numbering).

In some embodiments, the Fc region comprised in the antibody or antibody fragment may contain mutations that facilitate heterodimerization of the first Fc region with the second Fc region. Preferably, based on the Knob-in-Hole technology, the respective Knob mutations and Hole mutations are introduced in the first Fc region and the second Fc region. See, e.g., U.S. Pat. Nos. 5,731,168; 7,695,936; Ridgway et al, Prot Eng 9, 617-621 (1996) and Carter, J Immunol Meth 248, 7-15 (2001) or WO 96/27011 for this technique. The respective mutations may also be introduced in the first and second Fc regions based on the Innobody technique. See, e.g., PCT/CN2021/143141 for this technique.

In some embodiments, the antibodies or antibody fragments such as antigen binding fragments do not have a functional Fc region. For example, the antibodies or antigen binding fragments are Fab, Fab′, F(ab′) 2, and Fv fragments.

In some embodiments, antibodies or antibody fragments such as antigen binding fragments applicable in the present disclosure also comprise a mutation in the FR of the variable region, for example, a Q38D mutation in the light chain variable region and a Q39K mutation in the heavy chain variable region.

### Antibodies and Antigen Binding Fragments

A non-limiting antibody of the present disclosure can be an intact, four immunoglobulin chain antibody comprising two heavy chains and two light chains (also referred as “whole antibody” or “full-length antibody”). The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide.

In some embodiments, the antibody fragment is an antigen-binding fragment.

The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′) 2, and variants of these fragments. Thus, in some embodiments, an antibody or an antigen binding fragment thereof can be, e.g., a Fv, a Fd, a dAb, a bispecific antibody, a diabody, a linear antibody, a single-chain antibody molecule, a multi-specific antibody formed from antibody fragments, and any polypeptide that includes a binding domain which is, or is homologous to, an antigen binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antibody is a full-length antibody.

The antibodies and antibody fragments thereof can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antibody fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH—CH1) which, together with complementary light chain polypeptides, form a pair of antigen binding regions. Linear antibodies can be bispecific or monospecific.

One-armed antibodies can have a heavy chain and a light chain, and a heavy chain fragment comprising CH2 and CH3 domains of IgG. In some embodiments, a one-armed antibody is an antibody that only has one of the two antigen binding arms in a typical antibody. In some embodiments, a one-armed antibody comprises an antigen binding arm (e.g., VH+CH1 and VL+CL), and a Fc.

Antibodies and antibody fragments of the present disclosure can be modified in the Fc region to provide desired effector functions or serum half-life. In some embodiments, the Fc region can be modified to silence or decrease complement-dependent cytotoxicity (CDC) or antibody-dependent cellular cytotoxicity (ADCC).

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, the antibodies of the present disclosure are bispecific antibodies. The term “bispecific antibody” refers to an antibody comprising a first antigen-binding region and a second antigen-binding region, wherein the first antigen-binding region binds one antigen or epitope and the second antigen-binding region binds another antigen or another epitope. Therefore, the bispecific antibody according to the present disclosure has specificity to two different antigens, or to two different epitopes of one antigen. The bispecific antibody format comprises an IgG-like antibody (Fan et al. (2015) Journal of Hematology & Oncology. 8:130). The most common type of an IgG-like antibody comprises two Fab regions and two Fc regions, and the heavy and light chains of each Fab may be derived from a separate monoclonal antibody. The bispecific antibody of the present disclosure can be prepared using bispecific antibody formats or techniques known in the art. Specific exemplary bispecific formats that may be used in the context of the invention are described, for example, Labrijn, et al. Bispecific antibodies: a mechanistic review of the pipeline. Nature Reviews Drug Discovery, 2019, 18 (8): 1-24.

In some embodiments, the antibody of the invention comprises a heavy chain and a light chain, wherein the heavy chain comprises a heavy chain variable region and a heavy chain constant region, and the light chain comprises a light chain variable region and a light chain constant region, wherein one or more heavy chains comprise a modified heavy chain constant region or CH1 of the invention, and one or more light chains comprise a modified light chain constant region of the invention. In some embodiments, the antibody of the invention comprises two heavy chains and two light chains, wherein the heavy chain comprises a heavy chain variable region and a heavy chain constant region, and the light chain comprises a light chain variable region and a light chain constant region, wherein one or more heavy chains comprise a modified heavy chain constant region or CH1 of the invention, and one or more light chains comprise a modified light chain constant region of the invention.

In some embodiments, the antibody of the invention comprises two different heavy chains and/or two different light chains. In some embodiments, the two different light chains comprise two different light chain constant regions, respectively, for example, one comprises a Lambda light chain constant region and the other comprises a Kappa light chain constant region. In some embodiments, the antibody of the invention is a bispecific antibody, comprising one pair of heavy and light chains that specifically bind to first antigen and another pair of heavy and light chains that specifically bind to a second antigen.

The antibody of the invention, such as a full-length antibody or a multi-specific antibody, can bind to any target, such as an antigen, or a ligand or a receptor suitable for the antibody. In some embodiments, the antigen is a tumor-associated antigen (TAA). In some embodiments, the tumor-associated antigen is an immune checkpoint molecule. In some embodiments, the target or antigen is selected from FAP, CEA, p95 HER2, BCMA, EpCAM, MSLN, MCSP, HER-1, HER-2, HER-3, CD19, CD20, CD22, CD33, CD38, CD52Flt3, EpCAM, IGF-1R, FOLR1, Trop-2, CA-12-5, HLA-DR, MUC-1 (mucin), GD2, A33-antigen, PSMA, PSCA, transferrin-receptor, TNC (tendinin), CA-IX, CD3, B7H3, EGFR, Hel, cMET, Axl, GPRC5D, PD-1, PD-L1, CD47, immune activating molecules such as 4-1BB, CD40, OX40 et al.

In some embodiments, the target or antigen is selected from TROP2, HER2, or B7H3. In some embodiments, the bispecific antibodies of the invention bind two different epitopes of one antigen or bind two antigens. In some specific embodiments, the bispecific antibodies of the invention bind two epitopes of HER2.

In some embodiments, the antibody or antigen-binding fragment thereof of the invention is an antibody or antigen-binding fragment thereof that specifically binds HER2, which comprises a modified CH1 and a modified CL of the invention. In some embodiments, the anti-HER2 antibody or antigen-binding fragment thereof comprises 6 CDRs of antibodies known to specifically bind HER2 (e.g., Trastuzumab or Pertuzumab). In some embodiments, the anti-HER2 antibody or antigen-binding fragment thereof comprises 3 heavy chain variable region CDRs and/or 3 light chain variable region CDRs of an antibody known to specifically bind HER2 (e.g., Trastuzumab or Pertuzumab). In some embodiments, the anti-HER2 antibody or antigen-binding fragment thereof comprises the heavy chain variable region and/or light chain variable region of an antibody known to specifically bind HER2 (e.g., Trastuzumab or Pertuzumab). In some embodiments, the HER2 antibody is a full-length antibody. In some embodiments, the HER2 antibody is Trastuzumab or Pertuzumab, and comprising a modified CH1 and a modified CL of the invention.

In some embodiments, the anti-HER2 antibody is a bispecific antibody that specifically binds to 2 epitopes of HER2, and comprises two heavy chains and two light chains, wherein one heavy chain and one light chain comprises the modified CH1 and the modified CL of the invention and/or the other heavy chain and the other light chain comprise the modified CH1 and the modified CL of the invention. In some embodiments, one heavy chain and one light chain of the anti-HER2 bispecific antibody comprise the three heavy chain variable region CDRs of Trastuzumab and the three light chain variable region CDRs of Trastuzumab, respectively; and the other heavy chain and the other light chain comprise the three heavy chain variable region CDRs of Pertuzumab and the three light chain variable region CDRs of Pertuzumab, respectively. In some embodiments, one heavy chain and one light chain of the anti-HER2 bispecific antibody comprise the heavy chain variable region and the light chain variable region of Trastuzumab, respectively; and the other heavy chain and one light chain comprise the heavy chain variable region and light chain variable region of Pertuzumab, respectively.

In some embodiments, the heavy chain variable region of Trastuzumab comprises or consists of an amino acid sequence set forth in SEQ ID No:72 or 74, and the light chain variable region of Trastuzumab comprises or consists of an amino acid sequence set forth in SEQ ID No: 73 or 75. In some embodiments, the heavy chain variable region of Pertuzumab comprises or consists of an amino acid sequence set forth in SEQ ID NO:76, and the light chain variable region of Pertuzumab comprises or consists of an amino acid sequence set forth in SEQ ID NO: 77.

In some embodiments, an antibody or antigen-binding fragment thereof of the invention is an antibody or antigen-binding fragment thereof that specifically binds to TROP2, comprising the modified CH1 and modified CL of the invention. In some embodiments, the TROP2 antibody or antigen-binding fragment thereof comprises the 3 CDRs of the heavy chain variable region set forth in SEQ ID NO: 78 and/or the 3 CDRs of the light chain variable region set forth in SEQ ID NO: 79. In some embodiments, the TROP2 antibody or antigen binding fragment thereof comprises or consists of a heavy chain variable region and a light chain variable region, wherein the heavy chain variable region comprises or consists of an amino acid sequence set forth in SEQ ID NO: 78 and/or the light chain variable region comprises or consists of the amino acid sequence set forth in SEQ ID NO: 79. In some embodiments, the TROP2 antibody is a full-length antibody.

In some embodiments, an antibody or antigen-binding fragment thereof of the invention is an antibody or antigen-binding fragment thereof that specifically binds to B7H3, comprising the modified CH1 and modified CL of the invention. In some embodiments, the B7H3 antibody or antigen binding fragment thereof comprises the 3 heavy chain variable region CDRs of the heavy chain variable region set forth in SEQ ID No: 80 and/or the 3 light chain variable region CDRs of the light chain variable region set forth in SEQ ID No: 81. In some embodiments, the B7H3 antibody or antigen binding fragment thereof comprises or consists of a heavy chain variable region and a light chain variable region, wherein the heavy chain variable region comprises or consists of an amino acid sequence set forth in SEQ ID No: 80 and/or the light chain variable region comprises or consists of an amino acid sequence set forth in SEQ ID No: 81. In some embodiments, the B7H3 antibody is a full length antibody.

Any of the antibodies or antibody fragments described herein can be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antibody fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Antibody Drug Conjugates (ADC)

The antibodies or antibody fragments thereof described herein can be conjugated to a payload (e.g., a therapeutic agent or a drug). In some embodiments, the payload can be selected from cytotoxic drugs, biologically active proteins, synthetic polymers, enzymes, nucleic acids and fragments thereof e.g. DNA, or RNA. The payload can be covalently or non-covalently bind to the antibody or antigen-binding fragment or the antigen binding protein construct (e.g., a bispecific antibody). In some embodiments, the payload can be a small molecular (e.g., cytotoxic, radio-isotope, proteolysis targeting chimera), a nucleic acid (e.g., siRNA, antisense oligonucleotide), a protein or peptide (e.g., toxin, cytokine), or a lysosome-targeting chimaera (LYTAC).

In some embodiments, the payload is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the payload can include, but not limited to, cytotoxic reagents, such as chemo-therapeutic agents, immunotherapeutic agents and the like, antiviral agents or antimicrobial agents. In some embodiments, the payload to be conjugated can be selected from, but not limited to, MMAE (monomethyl auristatin E), MMAD (monomethyl auristatin D), or MMAF (monomethyl auristatin F).

In some embodiments, the payload is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the payload is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodepa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

In some embodiments, the antigen-binding construct is coupled to the payload via a cleavable linker e.g. a SPBD linker or a maleimidocaproyl-valine-citrulline-p-aminobenzyloxycarbonyl (VC) linker. In some embodiments, the antigen-binding construct is coupled to the drug via a non-cleavable linker e.g. a MCC linker formed using SMCC or sulfo-SMCC. Selection of an appropriate linker for a given ADC can be readily made by the skilled person having knowledge of the art and taking into account relevant factors, such as the site of attachment to the antigen binding construct, any structural constraints of the drug and the hydrophobicity of the drug (see, for example, review in Nolting, Chapter 5, Antibody-Drug Conjugates: Methods in Molecular Biology, 2013, Ducry (Ed.), Springer).

Linkers suitable for use in the present invention include, for example, cathepsin-degradable linkers such as Val-Cit linkers (e.g., vc-PAB), cBu-Cit linkers, and CX linkers; a non-cleavable linker such as an SMCC linker or an MD linker; acid-sensitive linkers, linkers of silicone structure, disulfide-carbamate linkers, MC-GGFG linkers, TRX linkers, galactoside-containing linkers, pyrophosphate linkers, near infrared-sensitive linkers, UV-sensitive linkers such as PC4AP (Antibody-drug conjugates: Recent advances in linkerchemistry, Su, Z., Xiao, D., Xie, F., Liu, L., Wang, Y., Fan, S., . . . . Li, S. (2021). Antibody-drug conjugates: Recent advances in linker chemistry. Acta Pharmaceutica Sinica B.). Linkers suitable for use in the present invention may also be a combination of one or more linkers, e.g., a cathepsin-degraded linker may be combined with other types of linkers to form a new linker. Thus, the term “linker” as used herein encompasses a single type of linker, or a combination of different types of linkers, provided that it is capable of coupling the antibody of the invention to a drug. In one embodiment, a linker suitable for use in the present invention is a maleimide-containing linker. Thus, in one embodiment, a linker suitable for use in the present invention is mc-VC-PAB or mc-GGFG.

A number of specific linker-toxin combinations have been described and may be used with the antigen binding constructs described herein to prepare ADCs in certain embodiments. Examples include, but are not limited to, cleavable peptide-based linkers with auristatins such as MMAE and MMAF, camptothecins such as SN-38, duocarmycins and PBD dimers; non-cleavable MC-based linkers with auristatins MMAF and MMAE; acid-labile hydrazone-based linkers with calicheamicins and doxorubicin; disulfide-based linkers with maytansinoids such as DM1 and DM4, and bis-maleimido-trioxyethylene glycol (BMPEO)-based linkers with maytansinoid DM1. Some payloads and linkers are described, e.g., in Peters & Brown, (2015) Biosci. Rep. e00225; Dosio et al., (2014) Recent Patents on Anti-Cancer Drug Discovery 9:35-65; US Patent Publication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the antibodies through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the ADCs described here. Various prepared linkers, linker components and toxins are commercially available or may be prepared using standard synthetic organic chemistry techniques. These methods are described e.g., in March's Advanced Organic Chemistry (Smith & March 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected antigen binding construct are also available commercially, for example, linker-toxins comprising DM1, DM4, MMAE, MMAF or Duocarmycin SA are available from Creative BioLabs (Shirley, N.Y.).

It is understood that the form of the pharmaceutically acceptable salt or the solvate of ADC is also applicable for the various embodiments as described in the present disclosure.

In some embodiments, the invention provides an antibody-drug conjugate of formula (I):

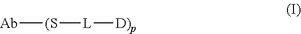

or a pharmaceutically acceptable salt or solvate thereof,
wherein:
Ab is an antibody or fragment (such as an antigen-binding fragment) thereof according to the invention, for example, an antibody or fragment (such as an antigen-binding fragment) thereof that specifically binds TROP2, HER2, and/or B7H3 (e.g., human TROP2, human HER2 and/or humanB7H3);
L is a linker;
D is a drug, such as an anti-tumor compound; and
p is an integer selected from 1 to 16, for example selected from 1-10, 1-9, 2-8, 4-10, 6-8, 3-7, 4-6, or 2-6, for example is 1, 2, 3, 4, 5, 6, 7, 8, 9, 10, or 12, preferably 4 or 6.

It will be understood that S in formula (I) is the sulfur from the antibody Ab.

In some embodiments, D in formula (I) of the present invention may be any anti-tumor compound, and is not particularly limited, as long as it has an anti-tumor effect and has a moiety which can be linked to a linker structure. The anti-tumor compound may be a pharmaceutically active compound that acts on a tumor. For a ADC, it is preferable that a part or whole of the linker is cleaved in a tumor cell to release the antitumor compound, thereby exhibiting an antitumor effect. When the linker is cleaved at the site linking to the drug, the antitumor compound is released in an unmodified form and exhibits its original antitumor effect. For example, D may be the payload defined herein.

In some embodiments, the anti-tumor compound is, for example, a cytotoxic agent, a topoisomerase I inhibitor; a small molecule tubulin inhibitor, such as camptothecins, auristatins, or maytansinoids, for example, Exatecan, Dxd, MMAE, MMAF, DM1 or DM4.

In some embodiments, -L- has a structure as follows:—Z-L₁-L₂-L₃-
wherein
Z is selected from

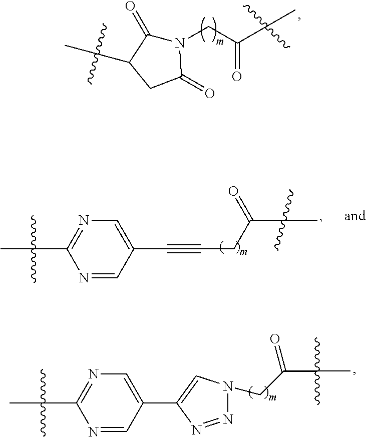

wherein m is an integer selected from 1 to 10, such as 1, 2, 3, 4, 5, 6, 7 or 8;
L1 is absent or selected from

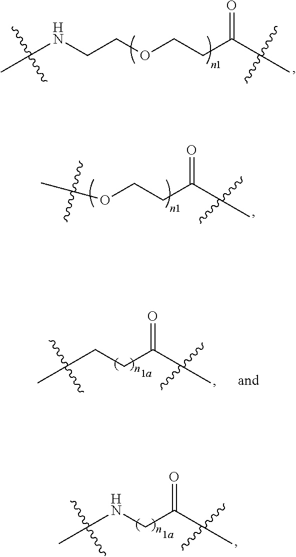

wherein each n1 is independently an integer selected from 0 to 12, such as 1, 2, 3, 4, 5, 6, 7 or 8; each n1a is independently an integer selected from 1 to 20, such as 1, 2, 3, 4, 5, 6, 7, 8, 9, 10, 12, 14, 16, 18, and 20;
L₂ is a peptide residue consisting of 2-8 amino acids; and
L₃ is absent or selected from:

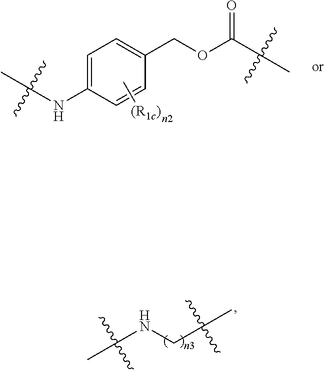

wherein R_{1c} is selected from H and C₁-C₆ alkyl; n2 is 1, 2, 3 or 4; and n3 is 1, 2, 3, 4, 5, or 6.

It should be understood, in the—Z-L₁-L₂-L₃- as described above, Z is linked to S of the Ab, and L₃ is linked to D.

In some embodiments, Z is selected from

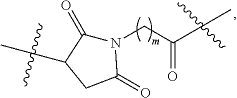

wherein m is 1, 2, 3, 4, 5, 6, 7 or 8.

In some embodiments, Z is selected from

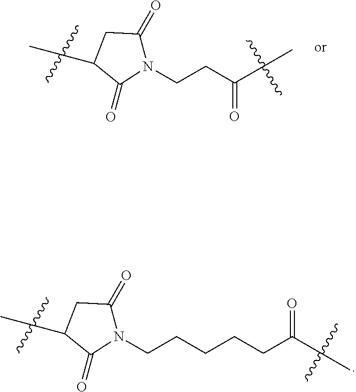 [SMILES: *C(=O)CCN1C(=O)CC(*)C1=O ; *C(=O)CCCCCN1C(=O)CC(*)C1=O]

In some embodiments, L1 is absent or selected from

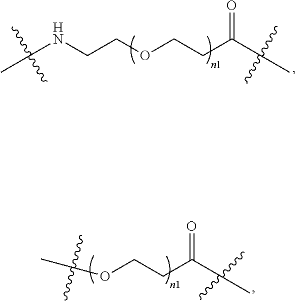

wherein each n1 is independently an integer selected from 0 to 12, such as 1, 2, 3, 4, 5, 6, 7 or 8.

In some embodiments, L₂ is a peptide residue consisting of 2, 3, 4, 5, 6, or 7 amino acids.

The amino acid constituting L₂ is not particularly limited, and is, for example, L- or D-amino acid, preferably L-amino acid. In addition to a-amino acids, it can also be amino acids with structures such as β-alanine, ε-aminocaproic acid, and γ-aminobutyric acid. In addition, it can also be non-natural amino acids such as N-methylated amino acids.

The amino acid constituting L₂ may be independently selected from phenylalanine (Phe; F), tyrosine (Tyr; Y), leucine (Leu; L), glycine (Gly; G), alanine (Ala; A), valine (Val; V), lysine (Lys; K), citrulline (Cit), serine (Ser; S), glutamic acid (Glu; E), aspartic acid (Asp; D), asparagine (Asn), isoleucine (Ile), arginine (Arg), proline (Pro), glutamine (Gln) and the like.

In some embodiments, L₂ is selected from -Val-Ala-, -Val-Cit-, -Ala-Ala-, -Ala-Cit-, -Ala-Lys-, -Ala-Val-, -Asn-Cit-, -Asp-Cit-, -Asn-Lys-, -Asp-Val-, -Cit-Ala-, -Cit-Asn-, -Cit-Asp-, -Cit-Cit-, -Cit-Lys-, -Cit-Ser-, -Cit-Val-, -Glu-Val-, -Glu-Gly-, -Ile-Cit-, -Ile-Pro-, -Ile-Val-, -Leu-Cit-, -Lys-Cit-, -Phe-Arg-, -Phe-Cit-, -Phe-Lys-, -Pro-Lys-, -Ser-Cit-, -Trp-Cit-, -Ala-Val-, -Val-Asp-, -Cit-Val-, -Val-Glu-, -Val-Lys-, -Gly-Gly-Gly-, -Gly-Gly-Arg-, -Phe-Lys-Gly-, -Leu-Lys-Gly-, -Leu-Leu-Gly-, -Glu-Val-Cit-, -Cit-Ala-Glu-, -Val-Lys-Gly-, -Val-Lys-Ala-, -Val-Gly-Gly-, -Val-Cit-Gly-, -Val-Gln-Gly-, -Val-Glu-Gly-, -Val-Lys-Gly-, -Val-Lys-Leu-, -Ala-Ala-Ala-, -Asn-Ala-Ala-, -Gly-Gly-Phe-Gly-, -Gly-Gly-Gly-Gly-, -Gly-Gly-Leu-Gly-, -Gly-Phe-Leu-Gly-, -Gly-Val-Lys-Gly-, -Ala-Leu-Ala-Leu-, -Gly-Phe-Leu-Gly-, -Ala-Leu-Ala-Leu-, -Gly-Phe-Gly-Gly-, -Val-Lys-Gly-Gly-, and -Pro-Leu-Gly-Leu-Ala-Gly-.

In some embodiments, L₂ is selected from -Val-Ala-, -Gly-Gly-Phe-Gly-, and -Val-Cit-.

It should be understood that L₂ is connected to Li or Z through the amino group of the left amino acid thereof, and is connected to L₃ through the carbonyl group of the right amino acid thereof, which is consistent with the explanation below.

In some embodiments, L₃ is selected from

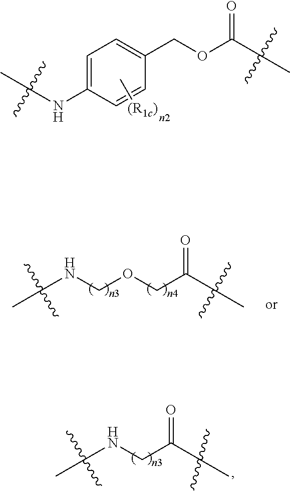

wherein R_{1c} is selected from H and C₁-C₆ alkyl; n2 is 1, 2, 3 or 4; and n3 and n4 are independently selected from 1, 2, 3, 4, 5, or 6.

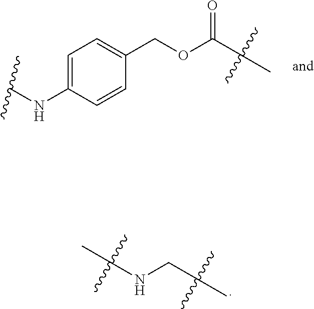

In some embodiments, L₃ is absent or selected from

It should be understood that L₃ is connected to L₂ through the left amino group of L₃ and to D through the group such as carbonyl group on the right, which is consistent with the explanation below.

In some embodiments, —Z-L₁-L₂-L₃-is selected from the structure as follows:

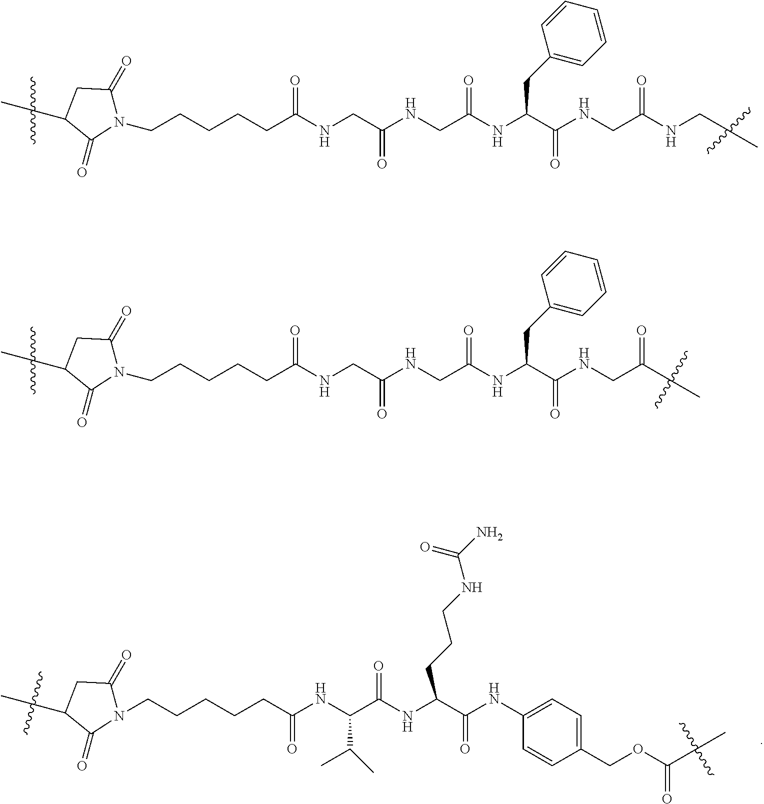 [SMILES: *CNC(=O)CNC(=O)[C@H](Cc1ccccc1)NC(=O)CNC(=O)CNC(=O)CCCCCN1C(=O)CC(*)C1=O ; *C(=O)CNC(=O)[C@H](Cc1ccccc1)NC(=O)CNC(=O)CNC(=O)CCCCCN1C(=O)CC(*)C1=O ; *C(=O)OCc1ccc(NC(=O)[C@H](CCCNC(N)=O)NC(=O)[C@@H](NC(=O)CCCCCN2C(=O)CC(*)C2=O)C(C)C)cc1]

In some embodiments, D has the structure represented by formula (D-1a) or formula (D-1b):

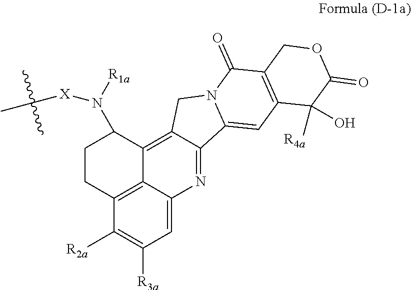

wherein R₁ₐ is selected from H and C₁-C₆ alkyl;
R2a is selected from H, halogen, C₁-C₆ alkyl, C₁-C₆ haloalkyl, —OR₅ₐ and —SR₅ₐ; R₃ₐ is selected from H, halogen, CN, C₁-C₆ alkyl, C₁-C₆ haloalkyl and —OR₅ₐ; or R₂ₐ and R₃ₐ together form —O(CR₆ₐR₇ₐ)ₙO—, where n is 1, 2, 3, or 4; and
R₄ₐ and R₅ₐ are independently selected from H and C₁-C₄ alkyl;
R₆ₐ and R₇ₐ are independently selected from H and halogen;
X is absent or selected from —O(CH₂)ₘC(O)—, m is 1, 2, 3, 4, 5, or 6, the right carbonyl of the group is connected to the nitrogen in formula (D-1a);

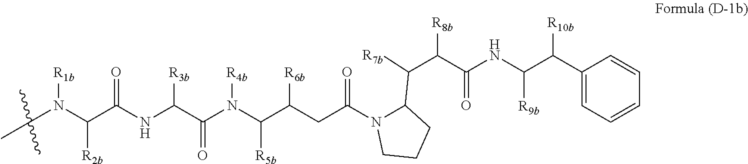

wherein R_{1b}, R_{2b}, R_{3b}, R_{4b}, R_{5b}, and R_{8b} are each independently selected from C₁₋₈ alkyl; preferably C₁₋₄ alkyl, such as methyl, ethyl, n-propyl, isopropyl, n-butyl, isobutyl or sec-butyl;
R_{6b} and R_{7b} are each independently selected from C₁₋₈ alkoxy, such as methoxy, ethoxy or propoxy;
R_{9b} is selected from C₁₋₈ alkyl and COOH; preferably C₁₋₄ alkyl, such as methyl, ethyl, n-propyl, isopropyl, n-butyl, isobutyl or sec-butyl; and
R_{10b} is selected from OH and H.

In some embodiments, Rᵢₐ is H; R₂ₐ is C₁-C₆ alkyl; R₃ₐ is halogen, preferably-F; R₄ₐ is C₁-C₄ alkyl, preferably ethyl.

In some embodiments, R_{1b}, R_{4b}, and R_{8b} are each independently selected from C₁₋₂ alkyl; preferably methyl;
R_{2b}, R_{3b}, and R_{5b} are each independently selected from C₃₋₄ alkyl;
R_{6b} and R_{7b} are each independently selected from C₁₋₂ alkoxy; and
R_{9b} is selected from C₁₋₄ alkyl and R_{10b} is OH; or R_{9b} is COOH and R_{10b} is H.

In some embodiments, D has the structure represented by Formula (D-2a) or Formula (D-2b):

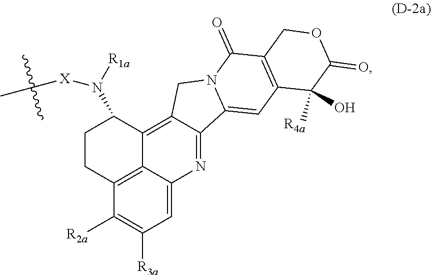

wherein Rᵢₐ, R₂ₐ, R₃ₐ, R₄ₐ, and X are as defined above; or

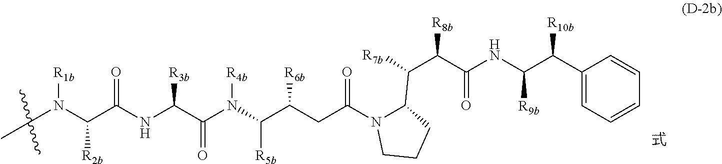

wherein R_{1b}, R_{2b}, R_{3b}, R_{4b}, R_{5b}, R_{6b}, R_{7b}, R_{8b}, R_{9b}, and R_{10b} are as defined above.

In some embodiments, D has the structure represented by Formula (D-3a) or Formula (D-3b):

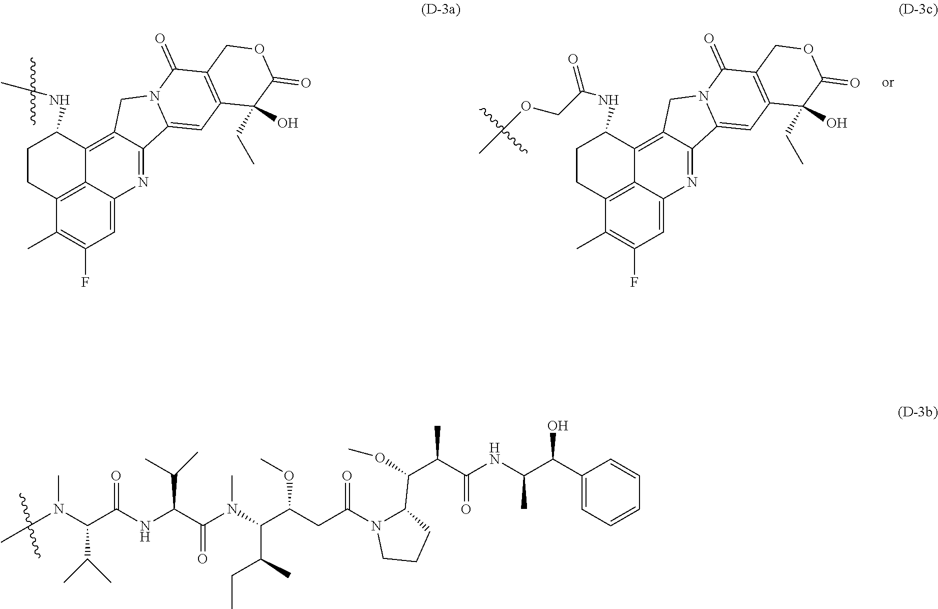 [SMILES: *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

It should be understood that, if not otherwise specified and not contradicted by the context, for the ADC of the present invention, the bond on the left side of the divalent group shown herein is connected to Ab or a group close to the Ab end, and the bond on the right side of the divalent group is connected to D or a group near the D end. For example, when L₂

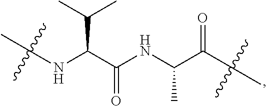 [SMILES: *N[C@H](C(=O)N[C@@H](C)C(*)=O)C(C)C]

wherein the amino group on the left is connected to L₁, and the carbonyl group on the right is connected to L₃;

In some embodiments, the antibody-drug conjugate has an average DAR of 2-10, 6-10, 4-8, 7-9, or 2-4.

In some embodiments, the antibody-drug conjugate is selected from:

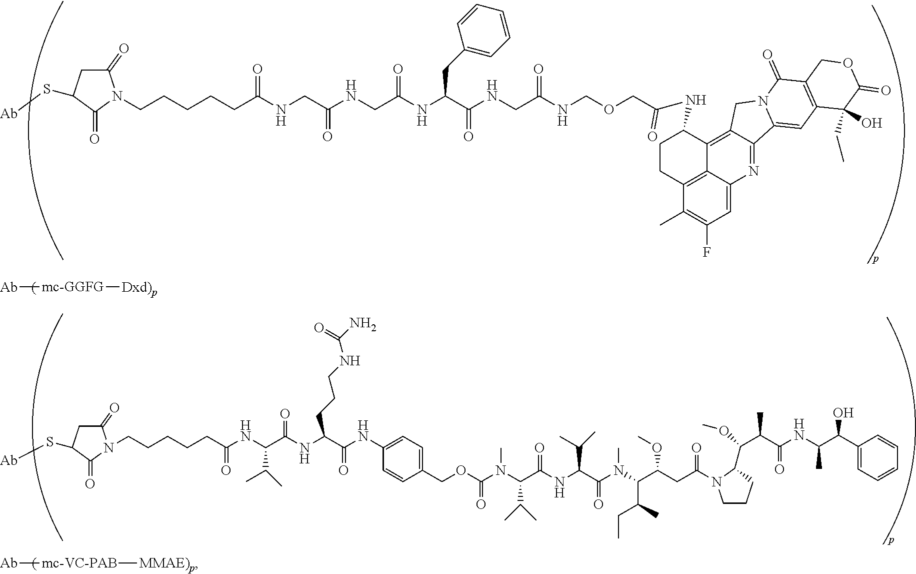

wherein Ab is an antibody or antigen-binding fragment of the invention, preferably; p is as defined above, preferably the antibody-drug conjugate has an average DAR of, for example, about 4 or 6.

It should be understood that the S atom connected to Ab in the above ADC is from Ab. The disulfide bond of Ab is opened under the action of a reducing agent such as TCEP to generate thiol (—SH), which is then connected to the terminal functional group of the linker such as the maleimide moiety.

Several specific examples of methods of preparing ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p≥5, may cause aggregation, insolubility, toxicity, or loss of cellular permeability of certain antibody-drug conjugates. In certain embodiments, the average drug loading for an antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the drug-to-antibody ratio (DAR) is about or at least 1, 2, 3, 4, 5, 6, 7, or 8. In some embodiments, the average DAR in the composition is about 1~ about 2, about 2~ about 3, about 3~ about 4, about 3~ about 5, about 4~ about 5, about 5~ about 6, about 6~ about 7, or about 7~ about 8.

In some embodiments, excess amounts of a reducing agent is added to the antibody or antibody fragment thereof to reduce the hinge region disulfide bonds. In some embodiments, the concentration of the antibody or antibody fragment thereof is higher than 0.1 mg/ml, higher than 0.2 mg/ml, higher than 0.3 mg/ml, higher than 0.4 mg/ml, higher than 0.5 mg/ml, higher than 1 mg/ml, higher than 2 mg/ml, higher than 3 mg/ml, higher than 4 mg/ml, higher than 5 mg/ml, higher than 10 mg/ml, higher than 20 mg/ml, higher than 30 mg/ml, higher than 40 mg/ml, or higher than 50 mg/ml. In some embodiments, the antibody concentration is lower than 0.1 mg/ml, lower than 0.2 mg/ml, lower than 0.3 mg/ml, lower than 0.4 mg/ml, lower than 0.5 mg/ml, lower than 1 mg/ml, lower than 2 mg/ml, lower than 3 mg/ml, lower than 4 mg/ml, lower than 5 mg/ml, lower than 10 mg/ml, lower than 20 mg/ml, lower than 30 mg/ml, lower than 40 mg/ml, or lower than 50 mg/ml. In some embodiments, the concentration of the antibody or antibody fragment thereof is 0.1-50 mg/ml, 1-50 mg/ml, 1-10 mg/ml or 1-5 mg/ml. In some embodiments, the reduction potential of the reducing agent is about −0.29 V. In some embodiments, the reduction potential of the reducing agent is between about −0.24 V and −0.32 V. In some embodiments, the reduction potential of the reducing agent is about −0.24 V, about −0.25, about −0.26, about −0.27, about −0.28, about −0.29, about −0.30, about −0.31, or about −0.32. In some embodiments, the reducing agent is added at more than 2, more than 3, more than 4, more than 5, more than 6, more than 7, more than 8, more than 9, more than 10, more than 15, more than 20, more than 25, more than 30, more than 40, or more than 50 equivalents per single antibody molecule. In some embodiments, the reducing agent is added at less than 2, less than 3, less than 4, less than 5, less than 6, less than 7, less than 8, less than 9, less than 10, less than 15, less than 20, less than 25, less than 30, less than 40, or less than 50 equivalents per single antibody molecule. In some embodiments, the reducing agent is added at 4-50 equivalents, or 8-30 equivalents per single antibody molecule. In some embodiments, the reducing agent is added at 10-30 equivalents per single antibody molecule. In some embodiments, the reducing agent is added at 10, 11, 12, 13, 14, 15, 20, 25, or 30 equivalents per single antibody molecule. In some embodiments, the reduction reaction takes place for more than 15 minutes, more than 30 minutes, more than 1 hour, more than 1.5 hours, more than 2 hours, more than 3 hours, more than 4 hours, more than 6 hours, more than 8 hours, more than 12 hours, or more than 24 hours. In some embodiments, the reduction reaction takes place for less than 15 minutes, less than 30 minutes, less than 1 hour, less than 1.5 hours, less than 2 hours, less than 3 hours, less than 4 hours, less than 6 hours, less than 8 hours, less than 12 hours, or less than 24 hours.

In some embodiments, the reducing agent is Tris (2-carboxyethyl) phosphine hydrochloride (TCEP), beta-mercaptoethanol (β-ME), dithiothreitol (DTT), dithioerythritol (DTE), tris-(hydroxymethyl)phosphine (THMP), tris(3-hydroxypropyl)phosphine (THPP), 2-Mercaptoethylamine·HCl (2-MEA), or Glutathione (GSH). In some embodiments, the reducing agent is selected from TCEP, THMP, and THPP. In some embodiments, the reducing agent is TCEP.

In some embodiments, excess TCEP (e.g., 10-30 equivalents, such as 10, 11, 12, 13, 14, 15, 20, 25, or 30 equivalents per single antibody molecule) is added to the antibody or antibody fragment thereof to reduce the hinge region disulfide bonds (e.g., at 2 mg/ml). In some embodiments, the reduction reaction takes place for more than 15 minutes, more than 30 minutes, more than 1 hour, more than 1.5 hours, more than 2 hours, more than 3 hours, more than 4 hours, more than 6 hours, more than 8 hours, more than 12 hours, or more than 24 hours. In some embodiments, the reduction reaction takes place for less than 15 minutes, less than 30 minutes, less than 1 hour, less than 1.5 hours, less than 2 hours, less than 3 hours, less than 4 hours, less than 6 hours, less than 8 hours, less than 12 hours, or less than 24 hours. In some embodiments, the reduction reaction takes place at room temperature. In some embodiments, the reduction reaction takes place in a phosphate-buffered saline (PBS) buffer. In some embodiments, the reduction reaction takes place in histidine buffer (e.g., 20 mM, pH=6.5) at 37° C. for 2 hours. In some embodiments, the disulfide bonds at hinge region were fully reduced. In some embodiments, more than 10%, more than 20%, more than 30%, more than 40%, more than 50%, more than 60%, more than 70%, more than 80%, more than 85%, more than 90% or more than 95% of the disulfide bonds at hinge region were reduced. In some embodiments, the reduction reaction takes place in a buffer system selected from Hepes, Histidine buffer, PBS, and MES. In some embodiments, the pH value of the buffer system is 5.5 to 8. In some embodiments, the reduction reaction is performed at a temperature of 0° C. to 37° C.

In some embodiments, excess linker-payload (e.g., 12 equivalents per single antibody molecule) was added to the antibody or antibody fragment that was reduced to form the ADC. In some embodiments, the linker-payload is added at more than 2, more than 3, more than 4, more than 5, more than 6, more than 7, more than 8, more than 9, more than 10, more than 11, more than 12, more than 13, more than 14, more than 15 equivalents per single antibody molecule. In some embodiments, the linker-payload is added at less than 2, less than 3, less than 4, less than 5, less than 6, less than 7, less than 8, less than 9, less than 10, less than 11, less than 12, less than 13, less than 14, less than 15, less than 20, less than 25, less than 30, less than 40 or less than 50 equivalents per single antibody molecule. In some embodiments, the linker-payload is added at 5-20 equivalents or 10-15 equivalents per single antibody molecule. In some embodiments, the linker-payload is added at 10, 11, 12, 13, 14, 15, or 20 equivalents per single antibody molecule. In some embodiments, the linking reaction takes place for more than 15 minutes, more than 30 minutes, more than 1 hour, more than 1.5 hours, more than 2 hours, more than 3 hours, more than 4 hours, more than 6 hours, more than 8 hours, more than 12 hours, or more than 24 hours. In some embodiments, the linking reaction takes place for less than 15 minutes, less than 30 minutes, less than 1 hour, less than 1.5 hours, less than 2 hours, less than 3 hours, less than 4 hours, less than 6 hours, less than 8 hours, less than 12 hours, or less than 24 hours. In some embodiments, the linking reaction takes place at room temperature. In some embodiments, the linking reaction takes place in a PBS buffer. In some embodiments, the linking reaction takes place in 10% Dimethylsulfoxide (DMSO) at 25° C. for 1.5 hours. In some embodiments, the linker is a maleimide linker. In some embodiments, the linker is a Maleimidocaproyl linker. In some embodiments, the payload is a drug. In some embodiments, the payload is a therapeutic agent. In some embodiments, the payload is a cytotoxic agent. In some embodiments, the linking reaction takes place in a buffer system selected from Hepes, Histidine buffer, PBS, and MES. In some embodiments, the pH value of the buffer system is 5.5 to 8. In some embodiments, the linker-payload comprises a maleimide moiety, bromide, or iodide. In some embodiments, the linking reaction is performed at a temperature of 0° C. to 37° C. In some embodiments, the payload comprises a diagnostic agent, a therapeutic agent or a labelling agent.

In some embodiments, excess N-acetylcysteine (e.g., 30 equivalents per single antibody molecule) was added to quench the unreacted linker-payload. In some embodiments, the quenching reaction takes place for more than 15 minutes, more than 30 minutes, more than 1 hour, more than 1.5 hours, more than 2 hours, more than 3 hours, more than 4 hours, more than 6 hours, more than 8 hours, more than 12 hours, or more than 24 hours. In some embodiments, the quenching reaction takes place for less than 15 minutes, less than 30 minutes, less than 1 hour, less than 1.5 hours, less than 2 hours, less than 3 hours, less than 4 hours, less than 6 hours, less than 8 hours, less than 12 hours, or less than 24 hours. In some embodiments, the quenching reaction takes place at room temperature. In some embodiments, the quenching reaction takes place in a PBS buffer. In some embodiments, the quenching reaction takes place for 20 minutes. In some embodiments, the quenching reaction takes place in a buffer system selected from Hepes, Histidine buffer, PBS, and MES. In some embodiments, the pH value of the buffer system is 5.5 to 8. In some embodiments, the quenching reaction is performed at a temperature of 0° C. to 37° C.

In some embodiments, the ADC is purified by a desalting column. In some embodiments, the ADC is purified by a Zeba™ Spin Desalting Column (e.g., 10 ml). In some embodiments, the remaining ADC is concentrated to obtain a solution of corresponding ADC.

In some embodiments, the resultant ADC are classified into D0, D2, D4, D6 and D8 based on the ratio between the antibody and payload molecules in the ADC, wherein DO refers to an antibody molecule that is not coupled to any payload molecules; D2 refers to the ADC in which two payload molecules are coupled to an antibody molecule; D4 refers to the ADC in which four payload molecules are coupled to an antibody molecule; D6 refers to the ADC in which six payload molecules are coupled to an antibody molecule; and D8 refers to the ADC in which eight payload molecules are coupled to an antibody molecule.

In some embodiments, the modified antibodies or antibody fragments thereof described herein can be used to produce site-specific DAR4 ADCs.

In some embodiments, the ADC described herein has an average drug-to-antibody ratio (DAR) of higher than 3, higher than 3.2, higher than 3.4, higher than 3.6, higher than 3.8, higher than 4, higher than 4.2, higher than 4.4, or higher than 4.6, as determined by HPLC. In some embodiments, the ADC described herein has an average DAR of lower than 3, lower than 3.2, lower than 3.4, lower than 3.6, lower than 3.8, lower than 4, lower than 4.2, lower than 4.4 or lower than 4.6, as determined by HPLC. In some embodiments, the ADC described herein has an average DAR of 3-5, 3.5-5, 3.5-4.5, 3.7-4.3, or 3.8-4.2. In some embodiments, the ADC described herein has an average DAR of 3.74-4 or 3.74-3.95. In some embodiments, the ADC described herein has an average DAR of 5.70-6 or 5.70-5.85.

In some embodiments, DAR4 species constitute more than 10%, more than 20%, more than 30%, more than 40%, more than 50%, more than 60%, more than 70%, more than 80%, more than 85%, more than 90% or more than 95% of the different DAR species in the ADC described herein. In some embodiments, DAR6 species constitute more than 10%, more than 20%, more than 30%, more than 40%, more than 50%, more than 60%, more than 70%, more than 80%, more than 85%, more than 90% or more than 95% of the different DAR species in the ADC described herein. In some embodiments, at least 90% of the resulting ADC products have a DAR of about 4. In some embodiments, at least 90% of the resulting ADC products have a DAR of about 6.

In some embodiments, the antibody or antigen-binding fragment thereof described herein or ADC derived therefrom has a purity of above 90%, above 91%, above 92%, above 93%, above 94%, above 95%, above 96%, above 97%, or above 98%, as determined by size exclusion chromatography (SEC). In some embodiments, the antibody or antibody fragment thereof as described herein or ADC derived therefrom has a hydrophobic interaction chromatography (HIC) retention time that is higher than 2 minutes, higher than 2.5 minutes, higher than 3 minutes, higher than 3.5 minutes, higher than 4 minutes, or higher than 4.5 minutes.

In some embodiments, the antibody or antigen-binding fragment thereof as described herein or ADC derived therefrom is stable in plasma. In some embodiments, the plasma stability can be measured by an in vitro plasma stability assay. In some embodiments, the antibody or antigen-binding fragment thereof as described herein or ADC derived therefrom is stable in plasma for at least 1 day, at least 2 days, at least 3 days, at least 4 days, or at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days.

In some embodiments, the antibody or antigen-binding fragment thereof as described herein or ADC derived therefrom can bind to a tumor cell. In some embodiments, the cell binding can be measured by an in vitro cell binding assay. In some embodiments, the cell binding can be measured by half maximal effective concentration (EC50). In some embodiments, the antibody or antigen-binding fragment thereof as described herein or ADC derived therefrom has a cell binding EC50 that is lower than 3, lower than 2.5, lower than 2, lower than 1.5, lower than 1, lower than 0.75, lower than 0.5, lower than 0.25, or lower than 0.15 nM.

In some embodiments, the antibody or antigen-binding fragment thereof as described herein or ADC derived therefrom can kill tumor cells. In some embodiments, the cell killing ability can be measured by an in vitro cytotoxicity assay. In some embodiments, the cytotoxicity can be measured by half maximal effective concentration (EC50). In some embodiments, the antibody or antigen-binding fragment thereof as described herein or ADC derived therefrom has a cytotoxicity EC50 that is lower than 3, lower than 2.5, lower than 2, lower than 1.5, lower than 1, lower than 0.75, lower than 0.5, lower than 0.25, or lower than 0.15 nM.

In some embodiments, the antibody or antigen-binding fragment thereof as described herein or ADC derived therefrom has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody or antigen-binding fragment thereof as described herein or ADC derived therefrom has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( Ti - T 0 ) / ( Vi - V 0 ) ] \times 100 \%$$

Ti is the average tumor volume in the treatment group on day i. T0 is the average tumor volume in the treatment group on day zero. Vi is the average tumor volume in the control group on day i. V0 is the average tumor volume in the control group on day zero.

### Methods of Making the Modified Antibodies or Antibody Fragment Thereof

Variants of the antibodies or antibody fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antibody fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

The antibodies generated by the mice have a full human VH, a full human VL, and mouse constant regions. In some embodiments, the human VH and human VL is linked to a human IgG constant regions (e.g., IgG1, IgG2, IgG3, and IgG4).

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric antibody will contain an amino acid sequence having at least 75%, 80%, 85%, 90%, 95%, 96%, 97%, 98%, or 99% percent identity with a sequence present in the light or heavy chain of the original antibody.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

Additional modifications to the antibodies or antibody fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In one aspect, the present disclosure provides a method of making an ADC, the method comprising:
(1) making a nucleic acid sequence encoding a heavy chain of an antibody or antibody fragment thereof, wherein a cysteine residue at position 220 is replaced with a non-cysteine amino acid, and/or making one or more of the following mutations: F126C, H168C, F170C, V173C, S134C, K133C, P130C, and A141C; and/or making a nucleic acid sequence encoding a light chain of an antibody or antibody fragment thereof by replacing a cysteine at position 214 with a non-cysteine amino acid, and/or making one or more of the following mutations: Q124C, T164C, S162C, F116C, F209C, I117C, and F118C;
(2) expressing the antibody or antibody fragment thereof;
(3) isolating the antibody or antibody fragment thereof;
(4) reacting the antibody or antibody fragment thereof with reducing agent; and
(5) reacting the antibody or antibody fragment thereof with a payload.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antibody fragments thereof and the ADC derived therefrom or the pharmaceutically acceptable salt or solvate thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof or the ADC derived therefrom disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, genitourinary cancer or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the cancer is NSCLC, ovarian cancer, melanoma, colorectal cancer, breast cancer, a hematological malignancy, head and neck cancer, gastrointestinal cancer, bladder cancer, or bone cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is breast cancer, lung cancer, pancreatic cancer, melanoma, oral cancer, mesothelioma, ovarian cancer, colorectal cancer, bladder cancer, gastroesophageal junction cancer, gastric cancer, non-small cell lung cancer, cervical cancer, brain cancer, skin cancer, multiple myeloma, Non-Hodgkin's lymphoma, solid tumors, lymphoma, epithelial neoplasms, soft tissue sarcoma, esophageal cancers, or CNS tumors.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, ADC, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment or the ADC derived therefrom is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment or ADC derived therefrom may vary, depending on, inter alia, patient history as well as other factors such as the type (and/or dosage) of antibody or ADC used.

Effective amounts and schedules for administering the antibodies, antibody fragments thereof, ADC derived therefrom, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody fragments thereof, ADC derived therefrom, antibody-encoding polynucleotides, and/or compositions disclosed herein, the route of administration, the particular type of antibodies, ADC, antibody-encoding polynucleotides, antigen binding fragments, and/or compositions disclosed herein used and other drugs being administered to the mammal. An exemplary daily dosage of an effective amount of an antibody, antibody fragment, or ADC derived therefrom is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, ADC derived therefrom, or pharmaceutical composition (e.g., any of the antibodies, antigen-binding fragments, ADC derived therefrom, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies, antibody fragments or ADC derived therefrom are administered in the same composition (e.g., a liquid composition). In some embodiments, at least one antibody, antigen-binding fragment, or ADC derived therefrom and at least one additional therapeutic agent are administered in the same composition (e.g., a liquid composition). In some embodiments, the at least one antibody, antigen-binding fragment or ADC derived therefrom and the at least one additional therapeutic agent are administered in two different compositions (e.g., a liquid composition containing at least one antibody, antigen-binding fragment or ADC derived therefrom and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, ADC derived therefrom or pharmaceutical composition (e.g., any of the antibodies, antigen-binding antibody fragments, ADC derived therefrom or pharmaceutical compositions described herein). In some embodiments, the one or more additional therapeutic agents and the at least one antibody, antigen-binding antibody fragment, ADC derived therefrom or pharmaceutical composition (e.g., any of the antibodies, antigen-binding antibody fragments, ADC derived therefrom or pharmaceutical compositions described herein) are administered to the subject such that there is an overlap in the bioactive period of the one or more additional therapeutic agents and the at least one antibody or antigen-binding fragment (e.g., any of the antibodies, antibody fragments or ADC derived therefrom described herein) in the subject.

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of OX40, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, a HER2 agonist and a OX40 agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, an anti-ICOS antibody, an anti-CD27 antibody, an anti-4-1BB antibody, and/or an anti-GITR antibody.

### Pharmaceutical Compositions and Routes of Administration

Also provided herein are pharmaceutical compositions that contain at least one (e.g., one, two, three, or four) of the antibodies, antibody fragments or ADC derived therefrom or the pharmaceutically acceptable salt or solvate thereof described herein. Two or more (e.g., two, three, or four) of any of the antibodies, antibody fragments or ADC derived therefrom or the pharmaceutically acceptable salt or solvate thereof described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody, antigen-binding fragment thereof or ADC derived therefrom can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antibody fragments or ADC derived therefrom described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies, antibody fragments or ADC derived therefrom can be formulated in aqueous solutions, preferably in physiologically-compatible buffers to reduce discomfort at the site of injection. The solution can contain formulatory agents such as suspending, stabilizing and/or dispersing agents. Alternatively, antibodies, antibody fragments or ADC derived therefrom can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50: ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies, antibody fragments thereof or ADC derived therefrom (e.g., any of the antibodies, antibody fragments or ADC derived therefrom described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies, antibody fragments or ADC derived therefrom described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies, antibody fragments or ADC derived therefrom described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies, antibody fragments thereof, or ADC derived therefrom, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody, antibody fragment or ADC derived therefrom in vivo.

The pharmaceutical compositions can be included in a container, pack, or dispenser together with instructions for administration. The disclosure also provides methods of manufacturing the antibodies, antigen binding fragments thereof or ADC derived therefrom for various uses as described herein.

### SPECIFIC EMBODIMENTS

The disclosure further refers to the following embodiments:
1. An antibody-drug conjugate (ADC) or the pharmaceutically acceptable salt or solvate thereof comprising a payload and an antibody or antibody fragment (e.g., antigen-binding fragment) thereof, for example, an antigen-binding fragment, or a pharmaceutically acceptable salt or solvate thereof, wherein the antibody or antibody fragment thereof comprises a non-native disulfide bond that is formed at the positions of a heavy chain amino acid residue and a light chain amino acid residue, wherein the sum of (1) the relative solvent accessible surface area (SASA) of the heavy chain amino acid residue side chain and (2) the relative SASA of the light chain amino acid residue side chain is below 65%, 60% or 55%, wherein the antibody or antibody fragment thereof does not have a disulfide bond between the amino acid residue at position 220 of the heavy chain constant region and the amino acid residue at position 214 of the light chain constant region, wherein the amino acid position is according to EU numbering.
2. The ADC or the pharmaceutically acceptable salt or solvate thereof of embodiment 1 or a pharmaceutically acceptable salt or solvate thereof, wherein the relative SASA of the heavy chain amino acid side chain is above 30% and the relative SASA of the light chain amino acid side chain is below 15%; or the relative SASA of the light chain amino acid side chain is above 30% and the relative SASA of the heavy chain amino acid side chain is below 15%.
3. The ADC or the pharmaceutically acceptable salt or solvate thereof of embodiment 1 or embodiment 2 or a pharmaceutically acceptable salt or solvate thereof, wherein the relative SASA of the heavy chain amino acid side chain is above 40% and the relative SASA of the light chain amino acid side chain is below 10%; or the relative SASA of the light chain amino acid side chain is above 40% and the relative SASA of the heavy chain amino acid side chain is below 10%.
4. The ADC or the pharmaceutically acceptable salt or solvate thereof of any one of embodiments 1-3 or a pharmaceutically acceptable salt or solvate thereof, wherein the relative SASA of each of the heavy chain and light chain amino acid side chains is below 20%, 25%, 30%, 35%, 40%, or 45%.
5. The ADC or the pharmaceutically acceptable salt or solvate thereof of any one of embodiments 1~4 or a pharmaceutically acceptable salt or solvate thereof, wherein (1) the amino acid residue at position 220 of a heavy chain constant region is a non-cysteine residue; or (2) the amino acid residue at position 214 of a light chain constant region is a non-cysteine residue, wherein the amino acid position is according to EU numbering.
6. The ADC or the pharmaceutically acceptable salt or solvate thereof of any one of embodiments 1-5 or a pharmaceutically acceptable salt or solvate thereof, wherein the non-native disulfide bond is resistant to tris(2-carboxyethyl) phosphine hydrochloride (TCEP) reduction and/or dithiothreitol (DTT) reduction.
7. The ADC or the pharmaceutically acceptable salt or solvate thereof of any one of embodiments 1-6 or a pharmaceutically acceptable salt or solvate thereof, wherein more than 90% or 95% of the non-native disulfide bonds remain intact when the antibody or antibody fragment thereof is reduced by 10-30 equivalents of TCEP per single antibody molecule.
8. The ADC or the pharmaceutically acceptable salt or solvate thereof of embodiment 7, wherein the concentration of the antibody or antibody fragment thereof is between 0.1-50 mg/ml during the reduction reaction.
9. The ADC or the pharmaceutically acceptable salt or solvate thereof of any one of embodiments 1-8 or a pharmaceutically acceptable salt or solvate thereof, wherein the non-native disulfide bond remains stable under reducing conditions that can reduce more than 90% of hinge region disulfide bonds of the antibody or antibody fragment.
10. The ADC or the pharmaceutically acceptable salt or solvate thereof of any one of embodiments 1-9 or a pharmaceutically acceptable salt or solvate thereof, wherein at least one non-cysteine amino acid residue in the heavy chain is mutated to a cysteine residue.
11. The ADC or the pharmaceutically acceptable salt or solvate thereof of embodiment 10 or a pharmaceutically acceptable salt or solvate thereof, wherein the non-cysteine amino acid residue is located at a position selected from the group consisting of: 126, 168, 170, 173, 134, 133, 130, and 141, wherein the amino acid position is according to EU numbering.
12. The ADC or the pharmaceutically acceptable salt or solvate thereof of any one of embodiments 1-11 or a pharmaceutically acceptable salt or solvate thereof, wherein the antibody or antibody fragment thereof comprises one or more of the following mutations in the heavy chain constant region: F126C, H168C, F170C, V173C, S134C, K133C, P130C, and A141C.
13. The ADC or the pharmaceutically acceptable salt or solvate thereof of any one of embodiments 1-12 or a pharmaceutically acceptable salt or solvate thereof, wherein the antibody or antibody fragment thereof comprises two heavy chains, each of which comprises one or more of the following mutations in the heavy chain constant region: F126C, H168C, F170C, V173C, S134C, K133C, P130C, and A141C.
14. The ADC or the pharmaceutically acceptable salt or solvate thereof of any one of embodiments 1-12 or a pharmaceutically acceptable salt or solvate thereof, wherein the antibody or antibody fragment thereof comprises two heavy chains, and only one heavy chain comprises one or more of the following mutations in the heavy chain constant region: F126C, H168C, F170C, V173C, S134C, K133C, P130C, and A141C.
15. The ADC or the pharmaceutically acceptable salt or solvate thereof of any one of embodiments 1-14 or a pharmaceutically acceptable salt or solvate thereof, wherein at least one non-cysteine amino acid in the light chain constant region of the antibody or antibody fragment thereof is mutated to a cysteine residue.
16. The ADC or the pharmaceutically acceptable salt or solvate thereof of embodiment 15 or a pharmaceutically acceptable salt or solvate thereof, wherein the at least one non-cysteine amino acid is located at a position selected from the group consisting of: 124, 164, 162, 116, 209, 117, and 118.
17. The ADC or the pharmaceutically acceptable salt or solvate thereof of any one of embodiments 1-16 or a pharmaceutically acceptable salt or solvate thereof, wherein the antibody or antibody fragment thereof comprises one or more of the following mutations in the light chain constant region: Q124C, T164C, S162C, F116C, F209C, I117C, and F118C.
18. The ADC or the pharmaceutically acceptable salt or solvate thereof of any one of embodiments 1-17 or a pharmaceutically acceptable salt or solvate thereof, wherein the antibody or antibody fragment thereof comprises two light chains, each of which comprises one or more of the following mutations in the light chain constant region: Q124C, T164C, S162C, F116C, F209C, I117C, and F118C.
19. The ADC or the pharmaceutically acceptable salt or solvate thereof of any one of embodiments 1-17 or a pharmaceutically acceptable salt or solvate thereof, wherein the antibody or antibody fragment thereof comprises two light chains, and only one light chain comprises one or more of the following mutations in the light chain constant region: Q124C, T164C, S162C, F116C, F209C, I117C, and F118C.
20. The ADC or the pharmaceutically acceptable salt or solvate thereof of any one of embodiments 1-19 or a pharmaceutically acceptable salt or solvate thereof, wherein the antibody or antibody fragment thereof comprises one or more of the below sets of mutations:
(1) F126C in the heavy chain and Q124C in the light chain;
(2) H168C in the heavy chain and T164C in the light chain;
(3) F170C in the heavy chain and T164C in the light chain;
(4) F170C in the heavy chain and S162C in the light chain;
(5) V173C in the heavy chain and S162C in the light chain;
(6) S134C in the heavy chain and F116C in the light chain;
(7) K133C in the heavy chain and F209C in the light chain;
(8) K133C in the heavy chain and I117C in the light chain;
(9) P130C in the heavy chain and F118C in the light chain; and
(10) A141C in the heavy chain and F116C in the light chain.
21. The ADC or the pharmaceutically acceptable salt or solvate thereof of any one of embodiments 1-20 or a pharmaceutically acceptable salt or solvate thereof, wherein the antibody or antibody fragment thereof comprises two pairs of heavy and light chains, of which only one pair of heavy and light chains comprise one of the below sets of mutations:
(1) F126C in the heavy chain and Q124C in the light chain;
(2) H168C in the heavy chain and T164C in the light chain;
(3) F170C in the heavy chain and T164C in the light chain;
(4) F170C in the heavy chain and S162C in the light chain;
(5) V173C in the heavy chain and S162C in the light chain;
(6) S134C in the heavy chain and F116C in the light chain;
(7) K133C in the heavy chain and F209C in the light chain;
(8) K133C in the heavy chain and I117C in the light chain;
(9) P130C in the heavy chain and F118C in the light chain; and
(10) A141C in the heavy chain and F116C in the light chain.
22. The ADC or the pharmaceutically acceptable salt or solvate thereof of any one of embodiments 1-20 or a pharmaceutically acceptable salt or solvate thereof, wherein the antibody or antibody fragment thereof comprises two pairs of heavy and light chains, of which each pair of heavy and light chains comprise one or more of the below sets of mutations:
(1) F126C in the heavy chain and Q124C in the light chain;
(2) H168C in the heavy chain and T164C in the light chain;
(3) F170C in the heavy chain and T164C in the light chain;
(4) F170C in the heavy chain and S162C in the light chain;
(5) V173C in the heavy chain and S162C in the light chain;
(6) S134C in the heavy chain and F116C in the light chain;
(7) K133C in the heavy chain and F209C in the light chain;
(8) K133C in the heavy chain and I117C in the light chain;
(9) P130C in the heavy chain and F118C in the light chain; and
(10) A141C in the heavy chain and F116C in the light chain.
23. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises F126C and each light chain comprises Q124C.
24. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises H168C and each light chain comprises T164C.
25. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises F170C and each light chain comprises T164C.
26. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises F170C and each light chain comprises S162C.
27. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises V173C and each light chain comprises S162C.
28. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises S134C and each light chain comprises F116C.
29. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises K133C and each light chain comprises F209C.
30. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises K133C and each light chain comprises I117C.
31. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises P130C and each light chain comprises F118C.
32. The ADC or the pharmaceutically acceptable salt or solvate thereof of embodiment 22 or a pharmaceutically acceptable salt or solvate thereof, wherein the antibody or antibody fragment thereof comprises two pairs of heavy and light chains, wherein each heavy chain comprises A141C and each light chain comprises F116C.
33. An ADC or the pharmaceutically acceptable salt or solvate thereof comprising a payload and an antibody or antibody fragment thereof, or a pharmaceutically acceptable salt or solvate thereof, wherein the antibody or antibody fragment thereof comprises a non-native disulfide bond that is formed at the positions of a heavy chain amino acid residue and a light chain amino acid residue, wherein the antibody or antibody fragment thereof does not have a disulfide bond between the amino acid residue at position 220 of the heavy chain constant region and the amino acid residue at position 214 of the light chain constant region, wherein when the antibody or antibody fragment thereof is exposed to a reducing condition that reduces more than 90% or 95% of the hinge disulfide bonds, more than 90% or 95% of the non-native disulfide bonds remain intact.
34. The ADC or the pharmaceutically acceptable salt or solvate thereof of any one of embodiments 1-33 or a pharmaceutically acceptable salt or solvate thereof, wherein the antibody or antibody fragment thereof comprises one or more of the below sets of heavy chain mutations:
(1) F126C and C220S;
(2) H168C and C220S;
(3) F170C and C220S;
(4) V173C and C220S;
(5) S134C and C220S;
(6) K133C and C220S;
(7) P130C and C220S; and
(8) A141C and C220S.
35. The ADC or the pharmaceutically acceptable salt or solvate thereof of any one of embodiments 1-34 or a pharmaceutically acceptable salt or solvate thereof, wherein the antibody or antibody fragment thereof comprises one or more of the below sets of light chain mutations:
(1) Q124C and C214S;
(2) T164C and C214S;
(3) S162C and C214S;
(4) F116C and C214S;
(5) F209C and C214S;
(6) I117C and C214S; and
(7) F118C and C214S.
36. The ADC or the pharmaceutically acceptable salt or solvate thereof of any one of embodiments 1-35 or a pharmaceutically acceptable salt or solvate thereof, wherein the antibody or antibody fragment thereof comprises one or more of the below sets of mutations:
(1) F126C and C220S in the heavy chain and Q124C and C214S in the light chain;
(2) H168C and C220S in the heavy chain and T164C and C214S in the light chain;
(3) F170C and C220S in the heavy chain and T164C and C214S in the light chain;
(4) F170C and C220S in the heavy chain and S162C and C214S in the light chain;
(5) V173C and C220S in the heavy chain and S162C and C214S in the light chain;
(6) S134C and C220S in the heavy chain and F116C and C214S in the light chain;
(7) K133C and C220S in the heavy chain and F209C and C214S in the light chain;
(8) K133C and C220S in the heavy chain and I117C and C214S in the light chain;
(9) P130C and C220S in the heavy chain and F118C and C214S in the light chain; and
(10) A141C and C220S in the heavy chain and F116C and C214S in the light chain.
37. The ADC or the pharmaceutically acceptable salt or solvate thereof of any one of embodiments 1-36 or a pharmaceutically acceptable salt or solvate thereof, wherein the antibody or antibody fragment thereof is a human or humanized antibody or antibody fragment thereof, a one-armed antibody, and/or a multi-specific antibody (e.g., a bispecific antibody).
38. The ADC or the pharmaceutically acceptable salt or solvate thereof of any one of embodiments 1-37 or a pharmaceutically acceptable salt or solvate thereof, wherein the antibody or antibody fragment thereof is a human IgG1, IgG2, IgG4 antibody or antibody fragment thereof.
39. The ADC or the pharmaceutically acceptable salt or solvate thereof of any one of embodiments 1-38 or a pharmaceutically acceptable salt or solvate thereof, wherein the antibody or antibody fragment thereof comprises a fragment crystallizable region (Fc region).
40. The ADC or the pharmaceutically acceptable salt or solvate thereof of any one of embodiments 1-39 or a pharmaceutically acceptable salt or solvate thereof, wherein the payload is a cytotoxic, cytostatic agent, biologically active proteins, synthetic polymers, enzymes, nucleic acids (e.g., DNA, or RNA) and fragments thereof.
41. The ADC or the pharmaceutically acceptable salt or solvate thereof of any one of embodiments 1-40 or a pharmaceutically acceptable salt or solvate thereof, wherein the drug-to-antibody ratio (DAR) is between 3.74 and 4.
42. The ADC or the pharmaceutically acceptable salt or solvate thereof of any one of embodiments 1-40 or a pharmaceutically acceptable salt or solvate thereof, wherein the drug-to-antibody ratio (DAR) is between 5.70 and 6.
43. The ADC or the pharmaceutically acceptable salt or solvate thereof of any one of embodiments 1-42 or a pharmaceutically acceptable salt or solvate thereof, wherein the payload is covalently bound to the antibody or antibody fragment thereof via a thiol-based conjugation.
44. An antibody or antibody fragment thereof comprising a non-native disulfide bond that is formed at the positions of a heavy chain amino acid residue and a light chain amino acid residue, wherein the sum of (1) the relative solvent accessible surface area (SASA) of the heavy chain amino acid residue side chain and (2) the relative SASA of the light chain amino acid residue side chain is below 65%, 60% or 55%, wherein the antibody or antibody fragment thereof does not have a disulfide bond between the amino acid residue at position 220 of the heavy chain constant region and the amino acid residue at position 214 of the light chain constant region, wherein the amino acid position is according to EU numbering.
45. The antibody or antibody fragment (e.g., antigen-binding fragment) thereof of embodiment 44, for example, an antigen-binding fragment, wherein the relative SASA of the heavy chain amino acid side chain is above 30% and the relative SASA of the light chain amino acid side chain is below 15%; or the relative SASA of the light chain amino acid side chain is above 30% and the relative SASA of the heavy chain amino acid side chain is below 15%.
46. The antibody or antibody fragment thereof of embodiment 44 or embodiment 45, wherein the relative SASA of the heavy chain amino acid side chain is above 40% and the relative SASA of the light chain amino acid side chain is below 10%; or the relative SASA of the light chain amino acid side chain is above 40% and the relative SASA of the heavy chain amino acid side chain is below 10%.
47. The antibody or antibody fragment thereof of any one of embodiments 44-46, wherein the relative SASA of each of the heavy chain and light chain amino acid side chains is below 20%, 25%, 30%, 35%, 40%, or 45%.
48. An antibody-drug conjugate or the pharmaceutically acceptable salt or solvate thereof comprising the antibody or antibody fragment thereof of any one of embodiments 44-47, covalently bound to a payload.
49. A nucleic acid encoding a light chain or a heavy chain of the antibody or antibody fragment thereof of any one of embodiments 1-48.
50. A vector comprising the nucleic acid of embodiment 49, wherein the nucleic acid is operably linked to a promoter.
51. A cell comprising the nucleic acid of embodiment 49 or the vector of embodiment 50.
52. A method of producing an antibody or an antibody fragment thereof, the method comprising
(a) culturing the cell of embodiment 51 under conditions sufficient for the cell to produce the antibody or the antibody fragment; and
(b) collecting the antibody or the antibody fragment produced by the cell.
53. A method of preparing an antibody-drug conjugate (ADC) or a pharmaceutically acceptable salt or solvate thereof, comprising the following steps:
(a) incubating the antibody or antibody fragment thereof of any one of embodiments 1-47 with a reductant to reduce native disulfide bonds within the antibody or antibody fragment thereof to generate reduced thiol groups;
(b) introducing an excess amount of a payload having a reactive group to react with the reduced thiol groups generated in step (a).
54. The method of embodiment 53, further comprising: (c) adding an effective amount of N-acetylcysteine to quench the excess payload, and then recovering the resultant antibody-drug conjugate.
55. The method of embodiment 53 or embodiment 54, wherein the payload comprises a maleimide group.
56. The method of any one embodiments 53-55, wherein the payload is linked to the antibody or antibody fragment thereof via a thiol-based conjugation.
57. The method of any one embodiments 53-56, wherein at least 50%, 60%, 70%, 80%, or 90% of the resulting ADC products have a drug-to-antibody ratio (DAR) of about 4.
58. The method of any one embodiments 53-56, wherein at least 50%, 60%, 70%, 80%, or 90% of the resulting ADC products have a drug-to-antibody ratio (DAR) of about 6.
59. A method of making a modified antibody or antibody fragment thereof, the method comprising:
(a) providing an antibody or antibody fragment thereof, wherein the antibody or antibody fragment thereof does not have a disulfide bond between the amino acid residue at position 220 of the heavy chain constant region and the amino acid residue at position 214 of the light chain constant region, wherein the amino acid position is according to EU numbering;
(b) identifying a heavy chain amino acid residue in the heavy chain constant region (CH1) and a light chain amino acid residue in the light chain constant region (CL), wherein the sum of (1) the relative solvent accessible surface area (SASA) of the heavy chain amino acid residue side chain and (2) the relative SASA of the light chain amino acid residue side chain is below 65%, 60% or 55%;
(c) substituting each of the identified heavy chain amino acid residue and light chain amino acid residue with a cysteine residue, wherein a non-native disulfide bond is formed between the two cysteine residues.
60. The method of embodiment 59, wherein the relative SASA of the heavy chain amino acid side chain is above 30% and the relative SASA of the light chain amino acid side chain is below 15% or the relative SASA of the light chain amino acid side chain is above 30% and the relative SASA of the heavy chain amino acid side chain is below 15%.
61. The method of embodiment 59 or embodiment 60, wherein the relative SASA of the heavy chain amino acid side chain is above 40% and the relative SASA of the light chain amino acid side chain is below 10%; or the relative SASA of the light chain amino acid side chain is above 40% and the relative SASA of the heavy chain amino acid side chain is below 10%.
62. The method of any one of embodiments 59-61, wherein the relative SASA of each of the heavy chain and light chain amino acid side chains is below 20%, 25%, 30%, 35%, 40%, or 45%.
63. The method of any one of embodiments 59-62, further comprising adding a payload to the modified antibody or antibody fragment thereof.
64. A method of treating a subject having cancer, the method comprising administering a therapeutically effective amount of a composition comprising the ADC or the pharmaceutically acceptable salt or solvate thereof of any one of embodiments 1-43 and 48, or a pharmaceutically acceptable salt or solvate thereof, to the subject.
65. The method of embodiment 64, wherein the subject has a solid tumor cancer.
66. The method of embodiment 64, wherein the cancer is breast cancer, lung cancer, pancreatic cancer, melanoma, oral cancer, mesothelioma, ovarian cancer, colorectal cancer, gastric cancer, cervical cancer, brain cancer, skin cancer, multiple myeloma, lymphoma, epithelial neoplasms, soft tissue sarcoma, esophageal cancers, or CNS tumors.
67. A method of decreasing the rate of tumor growth, the method comprising contacting a tumor cell with an effective amount of a composition comprising the ADC or the pharmaceutically acceptable salt or solvate thereof of any one of embodiments 1-43 and 48, or a pharmaceutically acceptable salt or solvate thereof.
68. A method of killing a tumor cell, the method comprising contacting a tumor cell with an effective amount of a composition comprising the ADC or the pharmaceutically acceptable salt or solvate thereof of any one of embodiments 1-43 and 48, or a pharmaceutically acceptable salt or solvate thereof.
69. A pharmaceutical composition comprising the ADC or the pharmaceutically acceptable salt or solvate thereof of any one of embodiments 1-43 and 48, or a pharmaceutically acceptable salt or solvate thereof, and a pharmaceutically acceptable carrier.

### Examples

The invention is further described in the following examples, which do not limit the scope of the invention described in the claims.

The following materials were used in the following examples:
1. Buffers

| Name | Compositions |
|---|---|
| Reaction buffer | 20 mM histidine buffer, pH 6.5 |
| ADC storage buffer | 20 mM histidine buffer, pH 6.5 |
| HIC mobile phase A | 1.5M ammonium sulfate, 50 mM |
| | sodium phosphate, pH 6.0 |
| HIC mobile phase B | 50 mM sodium phosphate, pH 6.0/ |
| | isopropanol (80:20 v/v) |
| SEC buffer | 0.2M PB, 0.25M KCl, 15% (v/v) IPA, |
| | pH 7.0 |

2. Reagents

| Name | Manufacturer, Cat#, Lot# |
|---|---|
| TFA (Trifluoroacetic acid) | Thermo Scientific, Lot# W1334149 |
| Histidine | Sigma, Lot# SLCG1233 |
| TCEP | Aldrich, Lot# SLBZ2552 |
| DMSO | Sigma, Lot# RNBJ3655 |
| Na₂HPO₄ | SCR, Lot# 10039-32-4 |
| NaH₂PO₄ | SCR, Lot# 13472-35-0 |
| NaCl | SCR, Lot# 7647-14-5 |
| (NH₄)₂SO₄ | Aladdin, Lot# H2007071 |
| Isopropanol | Wochem, Lot# 67-63-0 |
| Desalting column | Zeba ™ Spin Desalting Columns, 40K MWCO, 2/5/10 mL |
| Amicon 30K | Amicon, Cat# UFC503096, Lot# ROPB38507 |
| SEC column | TSK gel G3000SWXL, 7.8 mm × 30 cm, 5 μm |
| RP Column | Waters, BioResolve RP mAb Polyphenyl, 450 Å, |
| | 2.7 μm, 2.1 mm × 15 cm |
| HIC Column | TSKgel Butyl-NPR, 2.5 μm, 4.6 × 100 mm |

3. Equipment

| Equipment | |
|---|---|
| RP-HPLC | Waters, ACQuity H class |
| | UPLC, SZ-A2-DD-HPLC01 |
| SEC purity | Agilent, 1260, AS-146 |
| HIC chromatography | Agilent, 1260, AS-146 |

### Example 1: Design of Solvent Inaccessible Disulfide Bonds

First, cysteine residues corresponding to the natural heavy-light chain disulfides are mutated to non-cysteine residues, to remove the natural heavy-light chain disulfides.

Second, appropriate amino acid pairs on the binding interface of the heavy and light chains are selected and mutated into cysteine residues, to form new disulfide bonds. Amino acid pairs on the interface of heavy and light chains were screened and pairs with α-carbon atoms distance less than 10 Å, preferably less than 8 Å, and more preferably less than 6A, were selected, with side chains facing each other. The selected amino acid pairs are shown in the below table as “Candidate positions for non-native disulfide bond (CH1/CL)” (EU numbering).

To achieve the goal of DAR4, the new disulfide bonds should be hidden inside the protein structure domain in the solvent inaccessible region, so that the new disulfide bonds are not opened by reducing agents at normal reduction concentrations. The relative solvent accessible surface area (SASA) of the side chains for candidate amino acid pairs in a structure of a Fab fragment were calculated. The results are shown in the table below as “relative solvent accessible surface area (%).”

SASA was calculated by representing each atom as a collection of grid points distributed over a sphere whose radius is the sum of the atom's van der Waals radius and of a probe radius (1.4 Å). A grid point which is buried inside another atomic sphere contributes to the buried surface area. A grid point which is not buried inside another atomic sphere contributes to the exposed surface area. These results were calculated using the Discovery Studio software version 2022.

The relative residue SASA (in percentage) was calculated by the following formula: 100 times the residue SASA divided by the residue SASA of the fully exposed amino acid residue calculated using the extended Ala-X-Ala tripeptide, where X is the residue of interest.

TABLE 1
Candidate position pairs for non-native disulfide bond

| Candidate | | | | | |
|---|---|---|---|---|---|
| positions for | | | | | |
| non-native | | relative solvent | | Percentage of | Percentage of |
| disulfide bond | Disulfide | accessible surface | solvent | DAR4 ADC | DAR6 ADC |
| (CH1/CL) | Bond | area (%) | accessibility | (both arms) | (single arm) |
| F126/Q124 | ✓ | 15.541/4.676 | solvent | 87.55%~90.97% | 92.34% |
| H168/T164 | ✓ | 20.349/9.331 | inaccessible | 88.47% | NA |
| F170/T164 | ✓ | 1.351/9.331 | | 87.15% | 94.35% |
| F170/S162 | ✓ | 1.351/11.321 | | 87.63% | 90.98% |
| V173/S162 | ✓ | 30.901/11.321 | | 90.00% | NA |
| S134/F116 | ✓ | 0/5.743 | | 90.31% | 92.68% |
| K133/F209 | ✓ | 41.332/2.027 | | 94.16% | NA |
| K133/I117 | ✓ | 41.332/0.443 | | 92.25% | NA |
| P130/F118 | ✓ | 1.074/1.351 | | 98.62% | 94.49% |
| A141/F116 | ✓ | 0/5.743 | | 89.77% | NA |
| S134/T116 | ✓ | 4.988/45.96 | | 92.20% | NA |
| (lambda LC) | | | | | |
| P171/S162 | ✓ | 54.765/11.321 | solvent | NA | NA |
| T139/F116 | ✓ | 66.713/5.743 | accessible | NA | NA |
| V173/Q160 | ✓ | 30.901/53.342 | | NA | NA |
| S136/S114 | ✓ | 69.644/69.394 | | NA | NA |
| S132/F209 | ✓ | 116.527/2.027 | | NA | NA |
| S131/P119 | ✓ | 51.012/40.805 | | NA | NA |
| K133/K207 | ✓ | 41.332/31.926 | | NA | NA |
| S132/P119 | X | 116.527/40.805 | NA | NA | NA |
| S134/F118 | X | 0/1.351 | NA | NA | NA |
| F170/S174 | X | 1.351/0 | NA | NA | NA |
| Q175/T180 | X | 16.897/44.525 | NA | NA | NA |
| S181/T178 | X | 26.219/30.603 | NA | NA | NA |
| S183/T178 | X | 4.829/30.603 | NA | NA | NA |
| L128/F118 | X | 4.051/1.351 | NA | NA | NA |

In the below examples, modified antibodies were constructed based on candidate positions the above table. The modified antibodies were tested for disulfide bond formation using gel electrophoresis.

Conjugation experiments were performed to produce site-specific ADCs to test if the modified antibodies or antibody fragments thereof meet the requirements for constructing a DAR4 ADC or DAR6 ADC. Percentages of DAR4 ADC and DAR6 ADC are shown in the above table.

### Example 2: Antibody expression and purification

The nucleic acids encoding each chain of the modified antibody or antibody fragment thereof were synthesized, and were constructed on a pCDNA3.1 template plasmid.

The plasmids were filtered through a 0.22 μm filter and used for transient expression of the cells. Expi293 cells (Invitrogen) were cultured to reach the desired transfection volume and the cell density was adjusted to 3×10⁶ cells/mL the day before transfection. On the day of transfection, the cell density was adjusted to 3×10⁶ cells/ml again. The Opti-MEM medium (Gibco cat #31985-070) of the final volume 1/10 (v/v) was used as the transfection buffer. For monoclonal antibodies, the expression plasmids for (1) heavy chain, (2) light chain were mixed at 1:1 or other more proper ratios. For bispecific antibodies, the expression plasmids for (1) first heavy chain, (2) first light chain, (3) second heavy chain, (4) second light chain were mixed at 1:1:1:1 or other more proper ratios.

Appropriate amounts of Polyethyleneimine (PEI) (Polysciences, 23966) were added into the plasmid from the above step (the mass ratio of the plasmid to the PEI is 1:3), mixed uniformly, and incubated at room temperature for 10 min to obtain a DNA/PEI mixture. The DNA/PEI mixture was poured gently into HEK293 cells and mixed well, incubated for 24 h at 37° C. under 8% CO₂. After 24 hours, the culture was supplemented with VPA (Sigma, cat #P4543-100G) to a final VPA concentration of 2 mM and supplemented with 2% (v/v) Feed (1G/L Phytone Peptone+1G/L Difco Select Phytone) and the culture was continued for 6 days. The cells were collected, centrifuged at 4000 rpm for 30 minutes. The cell supernatant was filtered through a 0.45 μM filter and purified by Protein A affinity chromatography. Certain samples were further purified by ion exchange chromatography, KappaSelect affinity chromatography and LambdaFabSelect affinity chromatography.

For Protein A affinity chromatography purification, the sample was purified using a pre-packed column Hitrap Mabselect Sure (GE, 11-0034-95). The specific operation steps are: (1) equilibrating the packed column with 5 column volumes of equilibration solution (20 mM Tris, 150 mM NaCl, pH 7.2) before purification; (2) passing the collected sample through the column; (3) washing the packed column using 10 column volumes of balance solution to remove non-specific binding protein; (4) eluting with 5 column volumes of elution buffer (100 mM sodium citrate, pH 3.5); (5) collecting the eluate. 2 M Tris was added to neutralize the pH to 6.5. The purified antibodies were analyzed by liquid chromatography-mass spectrometry (LC-MS) to assess the pairing ratios.

For ion exchange chromatography purification, the sample was purified using Capto HiRes S 10/100 (GE, Cat. No. 29275879). The specific operation steps are: (1) exchanging the sample into low-salt PB buffer (10 mM phosphate, pH 6.0) using ultrafiltration concentrator tubes (MILLIPORE, Cat. No. UFC901096); (2) equilibrating the packed column with 5 column volumes of pH 6.0 low-salt PB buffer before purification; (3) passing the liquid-exchanged sample through the column; (4) washing the packed column with 10 column volumes of pH 6.0 low-salt PB buffer to remove non-specific binding protein; (5) eluting by linearly increasing the concentration of pH 6.0 high-salt PB buffer (10 mM phosphate, pH 6.0, 1 M NaCl) (linear gradient: the ratio of pH 6.0 high-salt PB buffer was increased from 30% to 100% over 30 column volumes); (6) collecting the eluted main peak; (7) exchanging the elute into PBS buffer (Gibco, Cat. No. 70011-044) using an ultrafiltration concentrator (MILLIPORE, Cat. No. UFC901096). The purified antibodies were analyzed by Liquid Chromatography-Mass Spectrometry (LC-MS) to assess the pairing ratios.

For KappaSelect affinity chromatography purification, the sample was purified using a prepacked column HiTrap KappaSelect (GE, 17-5458-11). The specific operation steps are as follows: (1) equilibrating the packed column with 5 column volumes of equilibration solution (PBS, pH7.4) before purification; (2) passing the sample through the column; (3) then washing the packed column with 10 column volumes of equilibration solution to remove non-specific binding protein; (4) eluting with 5 column volumes of elution buffer (0.1 M glycine buffer, pH 2.5) and (5) collecting the eluate. 2 M Tris was added to neutralize the pH to 6.5. The purified antibodies were analyzed by Liquid Chromatography-Mass Spectrometry (LC-MS) to assess the pairing ratios.

For LambdaFabSelect affinity chromatography purification, the sample was purified with a prepacked column HiTrap LambdaFabSelect (GE, 17-5482-11). The specific operation steps are: (1) equilibrating the packed column with 5 column volumes of equilibration solution (PBS, pH7.4) before purification; (2) passing the sample through the column, (3) washing the packed column with 10 column volumes of equilibration solution to remove non-specific binding protein; (4) eluting with 5 column volumes of elution buffer (0.1 M acetate buffer, pH 3.5) and (5) collecting the eluate. 2M Tris was added to neutralize the pH to 6.5. The purified antibodies were analyzed by Liquid Chromatography-Mass Spectrometry (LC-MS) to assess the pairing ratios.

### Example 3: Production of Site-Specific ADCs and General Analysis Procedures

The purified antibodies were linked to drugs according to the methods in Examples 4-6 below to obtain various ADCs. General procedures A-D were performed to characterize these ADCs.
General procedure A: DTT treatment to fully reduce all the interchain disulfide bonds. An antibody-drug conjugate solution (approximately 1 mg/mL, 50 μL) was mixed with a dithiothreitol (DTT) aqueous solution (1 M, 1.0 μL). The mixture was adjusted to pH 7.5 with Tris buffer (1 M, pH 8.5) and incubated at 37° C. for 30 minutes to prepare a sample that can be readily used in HPLC analysis.
General procedure B: Measurement of the ADC's drug-to-antibody ratio (DAR) using RP-HPLC analysis. RP-HPLC analysis was carried out under the measurement conditions in the table below. The signals were detected by dual wavelength: 280 nm/360 nm for ADCs carrying Dxd, and 280 nm/248 nm for ADCs carrying MMAE. Either 45 min or 30 min method can be used.

TABLE 2
RP-HPLC analysis parameters

| | |
|---|---|
| Equipment | Waters, ACQuity H class UPLC, SZ-A2-DD-HPLC01 |
| Column | Waters, BioResolve RP mAb Polyphenyl, |
| | 450 Å, 2.7 μm, 2.1 mm × 15 cm |
| Column Temp. | 70° C. |
| Mobile Phase | A: 0.1% TFA in H₂O, B: 0.1% TFA in ACN. |
| Flow Rate | 0.3 mL/min |
| Injection Amount | 10 μg |
| Detection Wavelength | 280 nm; 360 nm/248 nm |
| Gradient | Gradient |

| | | | | | |
|---|---|---|---|---|---|
| | Mobile | Mobile | | Mobile | Mobile |
| Time(min) | Phase A % | Phase B % | Time(min) | Phase A % | Phase B % |

For ADC with Dxd (45 min or 30 min):

| | | | | | |
|---|---|---|---|---|---|
| 0 | 75 | 25 | 0 | 70 | 30 |
| 3 | 75 | 25 | 3 | 70 | 30 |
| 5 | 70 | 30 | 23 | 50 | 50 |
| 35 | 58 | 42 | 24 | 10 | 90 |
| 40 | 10 | 90 | 25 | 10 | 90 |
| 41 | 75 | 25 | 26 | 70 | 30 |
| 45 | 75 | 25 | 30 | 70 | 30 |

For ADC with MMAE (45 min or 30 min):

| | | | | | |
|---|---|---|---|---|---|
| 0 | 75 | 25 | 0 | 70 | 30 |
| 3 | 75 | 25 | 3 | 70 | 30 |
| 33 | 46 | 54 | 23 | 50 | 50 |
| 34 | 10 | 90 | 24 | 10 | 90 |
| 40 | 10 | 90 | 25 | 10 | 90 |
| 41 | 75 | 25 | 26 | 70 | 30 |
| 45 | 75 | 25 | 30 | 70 | 30 |

General procedure C: Measurement of the ADC purity using SEC analysis. SEC analysis was carried out under the measurement conditions in the below table. The signals were detected by dual wavelength: 280 nm/360 nm for ADCs carrying Dxd, and 280 nm/248 nm for ADCs carrying MMAE.

TABLE 3
SEC analysis parameters

| | |
|---|---|
| Equipment | Agilent, 1260, AS-146 |
| Column | TSK gel G3000SWXL, 7.8 mm × 30 cm, 5 μm |
| Column Temp. | 25° C. |
| Mobile Phase | 0.2M PB, 0.25M KCl, 15% (v/v) IPA, pH 7.0 |
| Flow Rate | 0.75 mL/min |
| Injection Amount | 30 μg |
| Detection Wavelength | 280 nm, 360 nm/248 nm |
| Gradient | Isocratic |
| Time | 18 min |

General procedure D: Measurement of the drug distribution of ADCs using HIC analysis. An HIC analysis was carried out under the measurement conditions in the below table. The signals were detected by dual wavelength: 280 nm/360 nm for ADCs carrying Dxd, and 280 nm/248 nm for ADCs carrying MMAE.

TABLE 4
HIC analysis parameters

| Equipment | Agilent, 1260, AS-146 | |
|---|---|---|
| Column | TSKgel Butyl-NPR, 2.5 μm, 4.6 x 100 mm | |
| Column Temp. | 30° C. | |
| Mobile Phase | A: 1.5M ammonium sulfate, 25 mM sodium phosphate, pH 6.0 | |
| | B: 25 mM sodium phosphate, pH 6.0/isopropanol (80:20 v/v) | |
| Flow Rate | 0.65 mL/min | |
| Injection Amount | 30 μg | |
| Detection Wavelength | 280 nm, 360 nm/248 nm | |
| Time | 41 min | |
| | Time (min) | Buffer B (%) |
| Gradient | 0 | 0 |
| | 5 | 0 |
| | 30 | 80 |
| | 31 | 100 |
| | 35 | 100 |
| | 36 | 0 |
| | 41 | 0 |

### Example 4: Production of Site-Specific DAR4 ADCs and Analysis

The purified antibodies were linked to drugs according to the methods below to obtain ADCs.

### 1. HC_F126C/C220S, LC_Q124C/C214S (Both Arms)

Tris (2-carboxyethyl) phosphine hydrochloride (TCEP) aqueous solution (179 μL, 10 mM in H₂O, 20 equivalents per single antibody molecule) was added to a solution of humanized anti-HER2 antibody (trastuzumab (TZB)) with disulfide bond mutations at both arms (HC_F126C/C220S, LC_Q124C/C214S) (13.0 mg) in histidine buffer (5.2 mL, 20 mM, pH=6.5). The obtained mixture was incubated at 37° C. for 2 hours, so that the disulfide bonds at hinge region were fully reduced.

After cooling to room temperature, a solution of linker-payload (mc-GGFG-Dxd, 111.3 μL, 10 mg/mL in DMSO, 12 equivalents per single antibody molecule) was added and additional DMSO was added to reach a final concentration of 10% (DMSO/buffer, v/v). The reaction mixture was incubated at 25° C. for 1.5 hours. Subsequently, a 50 mM N-acetylcysteine aqueous solution (54 μL, pH=8.0, 30 equivalents per single antibody molecule) was added, and the mixture was stirred for another 20 minutes to quench the unreacted linker-payload. Thereafter, the mixture was desalted by a Zeba™ Spin Desalting Column (10 mL), and the remaining solution was concentrated to obtain a solution of corresponding ADC (12.05 mg, 2.51 mg/mL, 4.8 mL).

The obtained ADC is named as 126C_124C or TZB (126C_124C)-DXd or TZB-126-124-DXd.

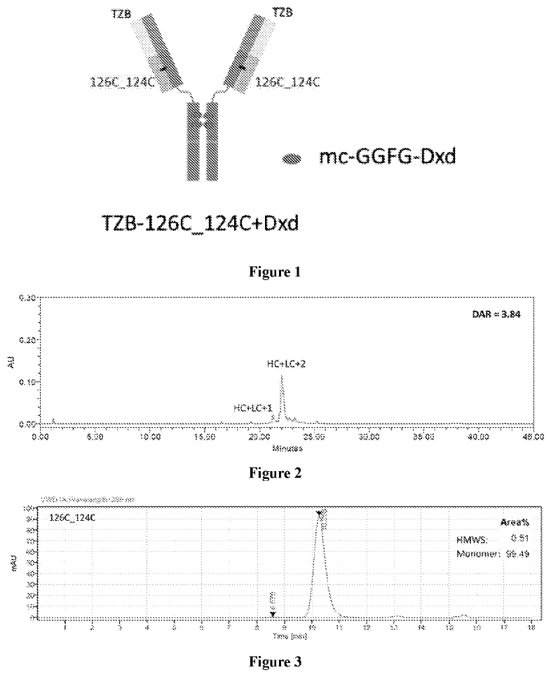

FIG. 1 is a schematic illustration of the ADC structure.

RP-HPLC analysis following the General procedure B indicated the drug-to-antibody ratio was 3.84 (FIG. 2).

SEC analysis following the General procedure C indicated the percentages of high-molecular-weight-species (HMWS) and monomer were 0.51%, 99.49%, respectively (FIG. 3).

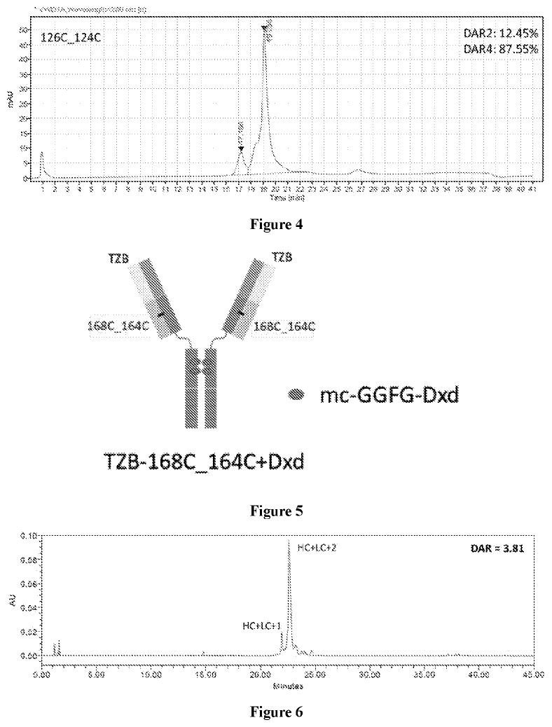

HIC analysis following the General procedure D showed the percentages of ADCs with different amounts of conjugated drugs was as follows: D2, 12.75%; D4, 87.55% (FIG. 4).

### 2. HC_H168C/C220S, LC_T164C/C214S (Both Arms)

TCEP aqueous solution (82.8 μL, 10 mM in H₂O, 30 equivalents per single antibody molecule) was added to a solution of humanized anti-HER2 antibody (trastuzumab) with disulfide bond mutation at both arms (HC_H168C/C220S, LC_T164C/C214S) (4.0 mg) in histidine buffer (2.0 mL, 20 mM, pH=6.5). The obtained mixture was incubated at 37° C. for 2 hours, so that the disulfide bonds at hinge region were fully reduced.

After cooling to room temperature, a solution of linker-payload (mc-GGFG-Dxd, 28.5 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (16.6 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (5 mL), and the remaining solution was concentrated to obtain a solution of corresponding ADC (3.43 mg, 2.45 mg/mL, 1.4 mL).

The obtained ADC is named as 168C_164C, or TZB (168C_164C)-DXd or TZB-168-164-DXd.

FIG. 5 is a schematic illustration of the ADC structure.

RP-HPLC analysis following the General procedure B indicated the drug-to-antibody ratio was 3.81 (FIG. 6).

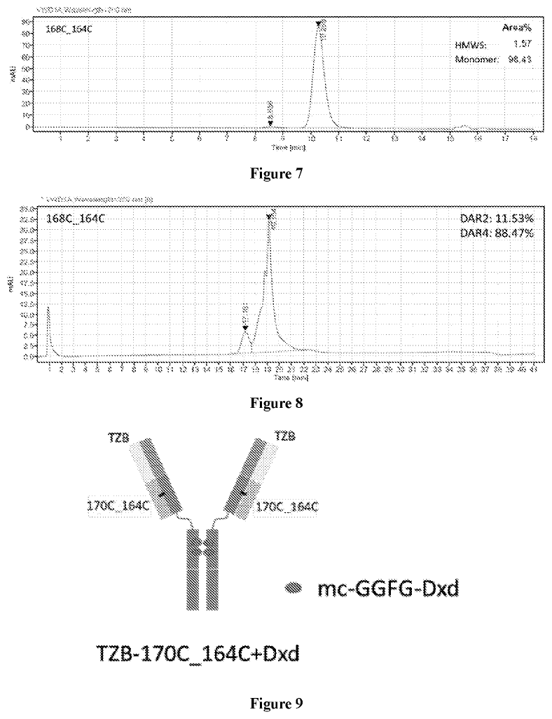

SEC analysis following the General procedure C indicated the percentages of high-molecular-weight-species (HMWS) and monomer were 1.57%, 98.43%, respectively (FIG. 7).

HIC analysis following the General procedure D showed the percentages of ADCs with different amounts of conjugated drugs was as follows: D2, 11.53%; D4, 88.47% (FIG. 8).

### 3. HC_F170C/C220S, LC_T164C/C214S (Both Arms)

TCEP aqueous solution (82.8 μL, 10 mM in H₂O, 30 equivalents per single antibody molecule) was added to a solution of humanized anti-HER2 antibody (trastuzumab) with disulfide bond mutation at both arms (HC_F170C/C220S, LC_T164C/C214S) (4.0 mg) in histidine buffer (2.0 mL, 20 mM, pH=6.5). The obtained mixture was then incubated at 37° C. for 2 hours, so that the disulfide bonds at hinge region were fully reduced.

After cooling to room temperature, a solution of linker-payload (mc-GGFG-Dxd, 28.5 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (16.6 μL, pH=8.0, 30 equivalents per single antibody molecule) was added, and the thus obtained mixture was further stirred at the same temperature for another 20 minutes to quench the unreacted linker-payload. Thereafter, the mixture was desalted by Zeba™ Spin Desalting Column (5 mL), and the remaining solution was concentrated to obtain a solution of corresponding ADC (3.48 mg, 2.90 mg/mL, 1.2 mL).

The obtained ADC is named as 170C_164C or TZB (170C_164C)-DXd or TZB-170-164-DXd.

FIG. 9 is a schematic illustration of the ADC structure.

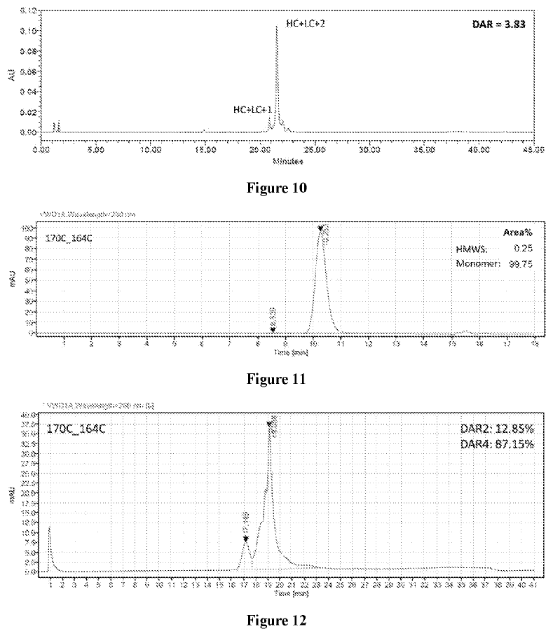

RP-HPLC analysis following the General procedure B indicated the drug-to-antibody ratio was 3.83 (FIG. 10).

SEC analysis following the General procedure C indicated the percentages of high-molecular-weight-species (HMWS) and monomer were 0.25%, 99.75%, respectively (FIG. 11).

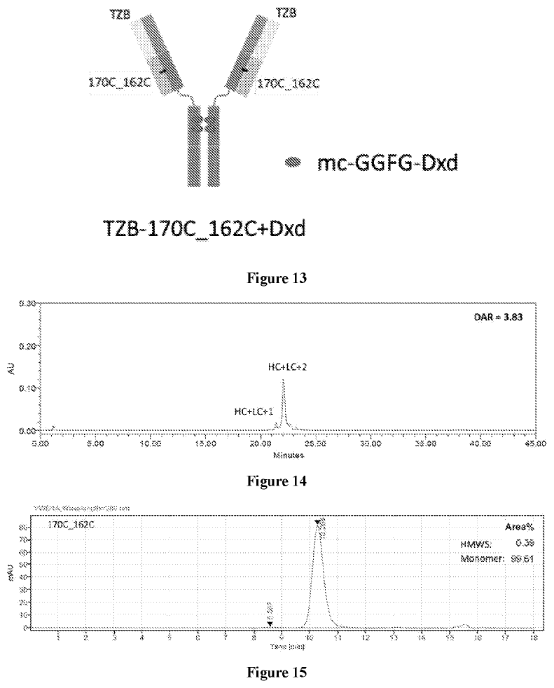

HIC analysis following the General procedure D showed the percentages of ADCs with different amounts of conjugated drugs was as follows: D2, 12.85%; D4, 87.15% (FIG. 12).

### 4. HC_F170C/C220S, LC_S162C/C214S (Both Arms)

TCEP aqueous solution (82.8 μL, 10 mM in H₂O, 30 equivalents per single antibody molecule) was added to a solution of humanized anti-HER2 antibody (trastuzumab) with disulfide bond mutation at both arms (HC_F170C/C220S, LC_S162C/C214S) (4.0 mg) in histidine buffer (2.0 mL, 20 mM, pH=6.5). The obtained mixture was then incubated at 37° C. for 2 hours, so that the disulfide bonds at hinge region were fully reduced.

After cooling to room temperature, a solution of linker-payload (mc-GGFG-Dxd, 28.5 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (16.6 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (5 mL), and the remaining solution was concentrated to obtain a solution of corresponding ADC (3.48 mg, 3.37 mg/mL, 1.0 mL).

The obtained ADC is named as 170C_162C, or TZB (170C_162C)-Dxd or TZB-170-162-Dxd.

FIG. 13 is a schematic illustration of the ADC structure.

RP-HPLC analysis following the General procedure B indicated the drug-to-antibody ratio was 3.83 (FIG. 14).

SEC analysis following the General procedure C indicated the percentages of high-molecular-weight-species (HMWS) and monomer were 0.39%, 99.61%, respectively (FIG. 15).

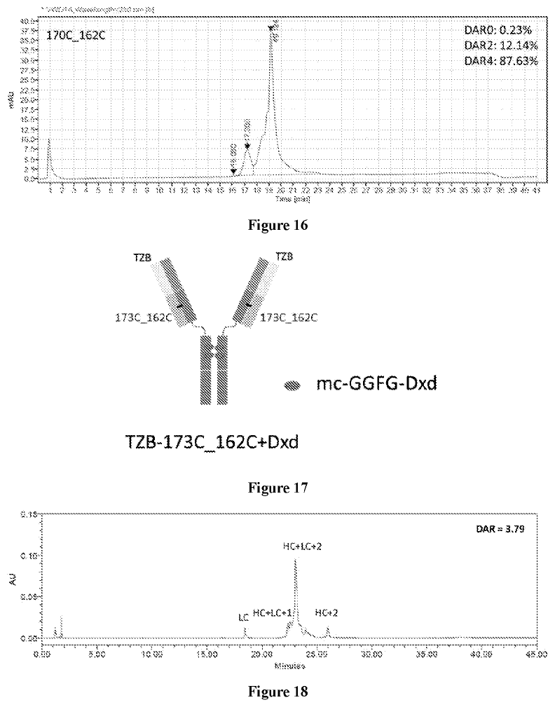

HIC analysis following the General procedure D showed the percentages of ADCs with different amounts of conjugated drugs was as follows: D0, 0.23%; D2, 12.14%; D4, 87.63% (FIG. 16).

5. HC_V173C/C220S, LC_S162C/C214S (both arms)

TCEP aqueous solution (13.8 μL, 10 mM in H₂O, 20 equivalents per single antibody molecule) was added to a solution of humanized anti-HER2 antibody (trastuzumab) with disulfide bond mutation at both arms (HC_V173C/C220S, LC_S162C/C214S) (1.0 mg) in histidine buffer (0.67 mL, 20 mM, pH=6.5). The obtained mixture was then incubated at 37° C. for 2 hours, so that the disulfide bonds at hinge region were fully reduced.

After cooling to room temperature, a solution of linker-payload (mc-GGFG-Dxd, 7.2 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (4.2 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (2 mL), and the remaining solution was concentrated to obtain a solution of corresponding ADC (0.78 mg, 2.55 mg/mL, 0.3 mL).

The obtained ADC is named as 173C_162C or TZB (173C_162C)-DXd.

FIG. 17 is a schematic illustration of the ADC structure.

RP-HPLC analysis following the General procedure B indicated the drug-to-antibody ratio was 3.79 (FIG. 18).

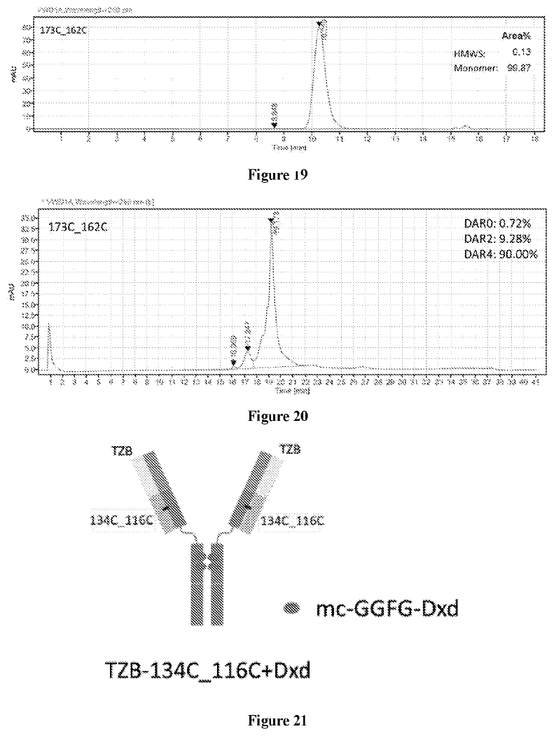

SEC analysis following the General procedure C indicated the percentages of high-molecular-weight-species (HMWS) and monomer were 0.13%, 99.87%, respectively (FIG. 19).

HIC analysis following the General procedure D showed the percentages of ADCs with different amounts of conjugated drugs was as follows: D0, 0.72%; D2, 9.28%; D4, 90.00% (FIG. 20).

### 6. HC_S134C/C220S, LC_F116C/C214S (Both Arms)

TCEP aqueous solution (310 μL, 10 mM in H₂O, 30 equivalents per single antibody molecule) was added to a solution of humanized anti-HER2 antibody (trastuzumab) with disulfide bond mutation at both arms (HC_S134C/C220S, LC_F116C/C214S) (15.0 mg) in histidine buffer (6.0 mL, 20 mM, pH=6.5). The obtained mixture was then incubated at 37° C. for 2 hours, so that the disulfide bonds at hinge region were fully reduced.

After cooling to room temperature, a solution of linker-payload (mc-GGFG-Dxd, 128.4 μL, 10 mg/mL in DMSO, 12 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (62 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (10 mL), and the remaining solution was concentrated to obtain a solution of corresponding ADC (13.35 mg, 2.67 mg/mL, 5.0 mL).

The obtained ADC is named as 134C_116C or TZB (134C_116C)-DXd or TZB-134-116-DXd.

FIG. 21 is a schematic illustration of the ADC structure.

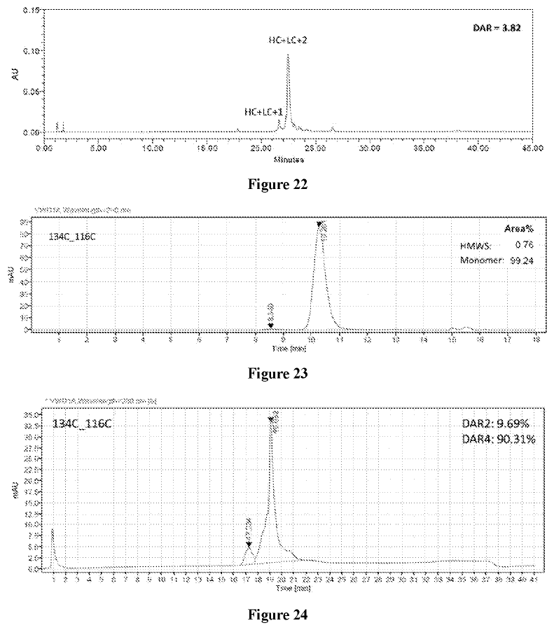

RP-HPLC analysis following the General procedure B indicated the drug-to-antibody ratio was 3.82 (FIG. 22).

SEC analysis following the General procedure C indicated the percentages of high-molecular-weight-species (HMWS) and monomer were 0.76%, 99.24%, respectively (FIG. 23).

HIC analysis following the General procedure D showed the percentages of ADCs with different amounts of conjugated drugs was as follows: D2, 9.69%; D4, 90.31% (FIG. 24).

### 7. HC_F126C/C220S, LC_Q124C/C214S (Both Arms)

TCEP aqueous solution (5.1 μL, 10 mM in H₂O, 25 equivalents per single antibody molecule) was added to a solution of an anti-B7H3 antibody with disulfide bond mutation at both arms (HC_F126C/C220S, LC_Q124C/C214S) (0.3 mg) in histidine buffer (0.2 mL, 20 mM, pH=6.5). The obtained mixture was incubated at 37° C. for 2 hours, so that the disulfide bonds at hinge region were fully reduced.

After cooling to room temperature, a solution of linker-payload (mc-VC-PAB-MMAE, 4.05 μL, 10 mg/mL in DMSO, 15 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (1.2 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

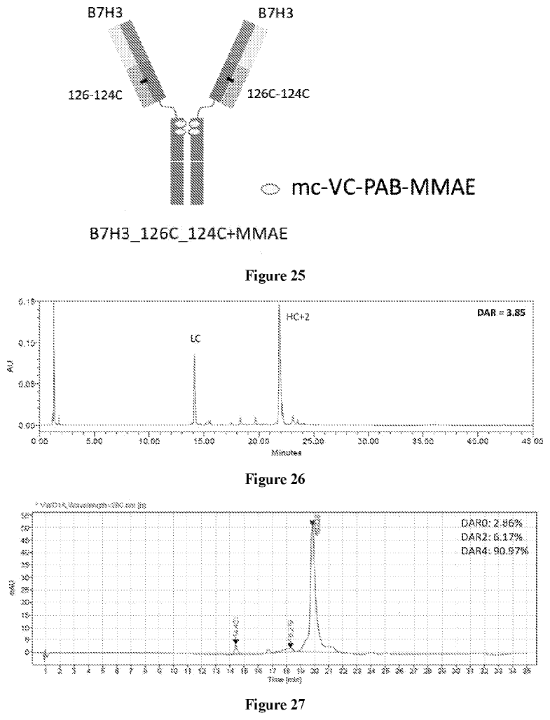

FIG. 25 is a schematic illustration of the ADC structure.

The resulting ADC product was first treated with DTT following the General procedure A, and then analyzed by RP-HPLC. The RP-HPLC analysis following the General procedure B indicated the drug-to-antibody ratio was 3.85 (FIG. 26).

HIC analysis following the General procedure D showed the percentages of ADCs with different amounts of conjugated drugs was as follows: D0, 2.86%; D2, 6.17%; D4, 90.97% (FIG. 27).

### 8. HC_F126C/C220S, LC_Q124C/C214S (Both Arms)

TCEP aqueous solution (6.9 μL, 10 mM in H₂O, 20 equivalents per single antibody molecule) was added to a solution of an anti-Trop2 antibody with disulfide bond mutation at both arms (HC_F126C/C220S, LC_Q124C/C214S) (0.5 mg) in histidine buffer (0.33 mL, 20 mM, pH=6.5). The obtained mixture was incubated at 37° C. for 2 hours, so that the disulfide bonds at hinge region were fully reduced.

After cooling to room temperature, a solution of linker-payload (mc-VC-PAB-MMAE, 5.44 μL, 10 mg/mL in DMSO, 12 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (2.1 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (2 mL), and the remaining solution was concentrated to obtain a solution of corresponding ADC (0.3 mg, 2.96 mg/mL, 0.1 mL).

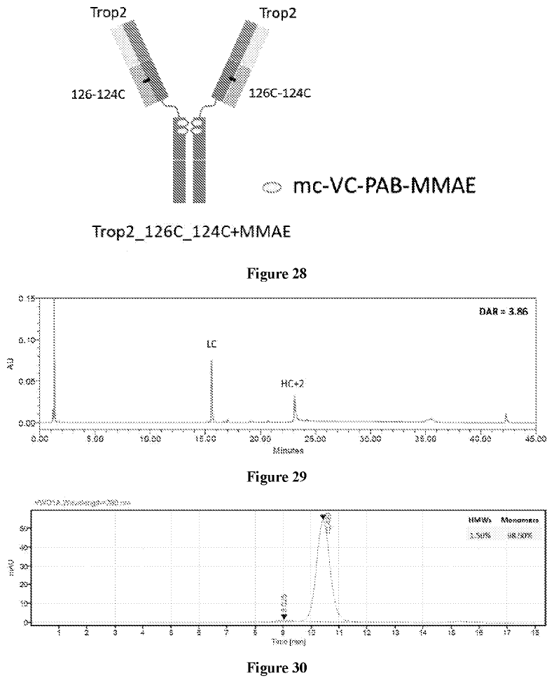

FIG. 28 is a schematic illustration of the ADC structure.

The resulting ADC product was first treated with DTT following the General procedure A, and then analyzed by RP-HPLC. The RP-HPLC analysis following the General procedure B indicated the drug-to-antibody ratio was 3.86 (FIG. 29).

SEC analysis following the General procedure C indicated the percentages of high-molecular-weight-species (HMWS) and monomer were 1.50%, 98.50%, respectively (FIG. 30).

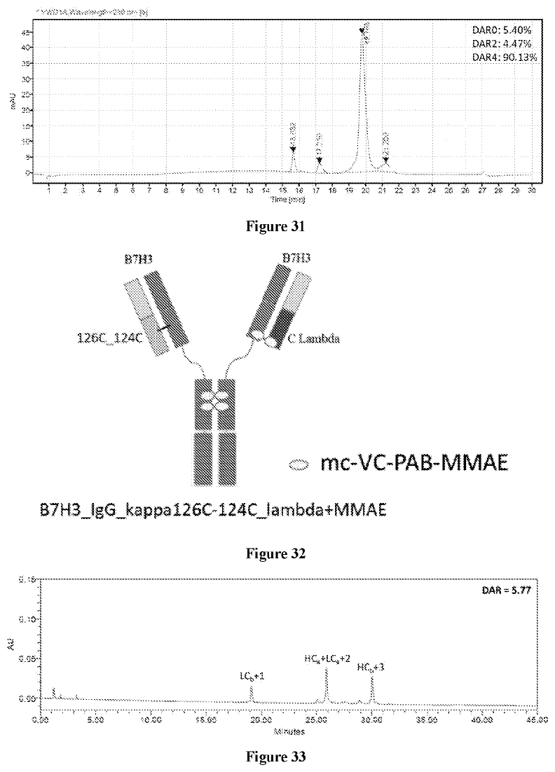

HIC analysis following the General procedure D showed the percentages of ADCs with different amounts of conjugated drugs was as follows: D0, 5.40%; D2, 4.47%; D4, 90.13% (FIG. 31).

### 9. HC_F126C/C220S, LC_Q124C/C214S (Single Arm)

TCEP aqueous solution (3.4 μL, 10 mM in H₂O, 10 equivalents per single antibody molecule) was added to a solution of an anti-B7H3 antibody with a disulfide bond mutation at only one Fab arm (HC_F126C/C220S, LC_Q124C/C214S) (0.5 mg) in histidine buffer (0.33 mL, 20 mM, pH=6.5). The obtained mixture was incubated at 37° C. for 2 hours.

After cooling to room temperature, a solution of linker-payload (mc-VC-PAB-MMAE, 5.31 μL, 10 mg/mL in DMSO, 15 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (2.1 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (2 mL), and the remaining solution was concentrated to obtain a solution of corresponding ADC (0.3 mg, 3.15 mg/mL, 0.1 mL).

FIG. 32 is a schematic illustration of the ADC structure.

RP-HPLC analysis following the General procedure B indicated the drug-to-antibody ratio was 5.77 (FIG. 33).

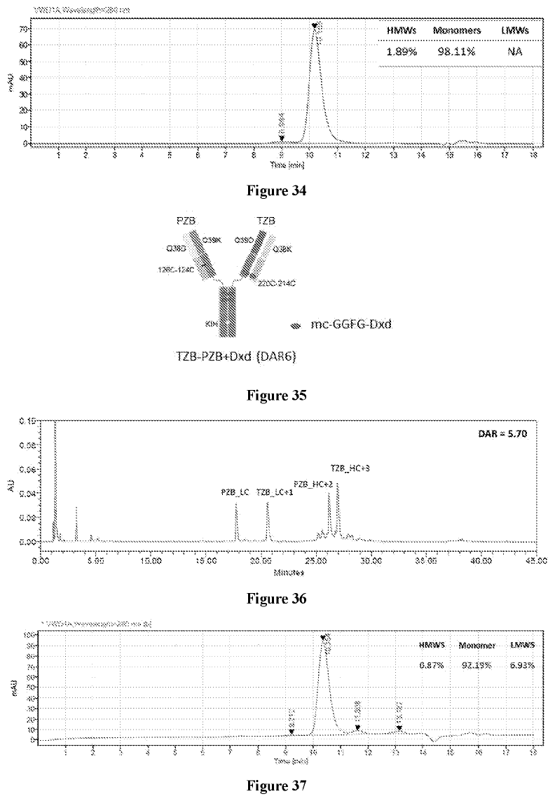

SEC analysis following the General procedure C indicated the percentages of high-molecular-weight-species (HMWS) and monomer were 1.89%, 98.11%, respectively (FIG. 34).

### 10. HC_F126C/C220S, LC_Q124C/C214S (Single Arm)

TCEP aqueous solution (13.8 μL, 10 mM in H₂O, 10 equivalents per single antibody molecule) was added to a solution of a bispecific antibody (Trastuzumab-Pertuzumab, or TZB-PZB) with a disulfide bond mutation at the PZB arm (HC_F126C/C220S, LC_Q124C/C214S) (2.0 mg) in histidine buffer (0.8 mL, 20 mM, pH=6.5). The obtained mixture was incubated at 37° C. for 2 hours.

After cooling to room temperature, a solution of linker-payload (mc-GGFG-Dxd, 17.2 μL, 10 mg/mL in DMSO, 12 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (8.3 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (10 mL), and the remaining solution was concentrated to obtain a solution of corresponding ADC (1.46 mg, 2.43 mg/mL, 0.6 mL).

FIG. 35 is a schematic illustration of the ADC structure.

The resulting ADC product was first treated with DTT following the General procedure A, and then analyzed by RP-HPLC. The RP-HPLC analysis following the General procedure B indicated the drug-to-antibody ratio was 5.70 (FIG. 36).

SEC analysis following the General procedure C indicated the percentages of high-molecular-weight-species (HMWS), monomer, and low-molecular-weight-species (LMWS) were 0.87%, 92.19%, and 6.93%, respectively (FIG. 37).

### 11. HC_132C/C220S, LC_119C/C214S (Both Arms)

TCEP aqueous solution (2.76 μL, 10 mM in H₂O, 20 equivalents per single antibody molecule) was added to a solution of Trastuzumab (TZB) with a disulfide bond mutation (HC_132C/C220S, LC_119C/C214S) (0.2 mg) in histidine buffer (80 μL, 20 mM, pH=6.5). The obtained mixture was incubated at 37° C. for 2 hours.

After cooling to room temperature, a solution of linker-payload (mc-GGFG-Dxd, 1.42 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.8 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

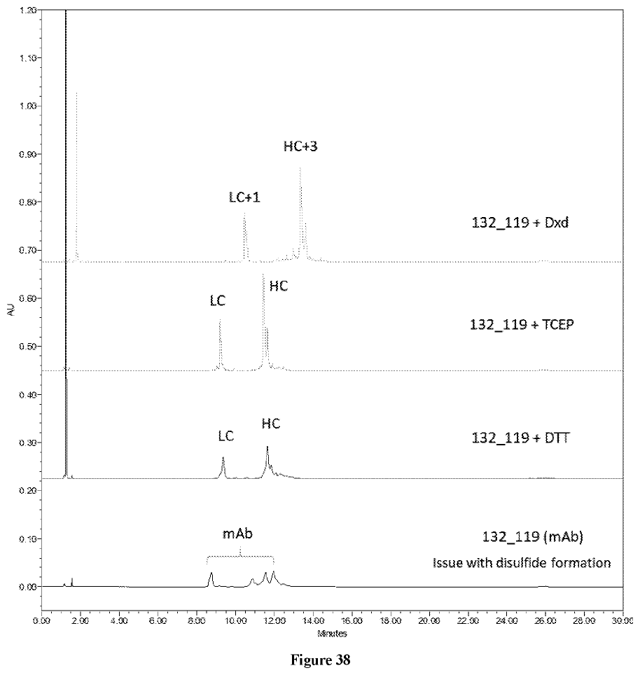

The RP-HPLC analysis following the General procedure B indicated that the modified antibody failed to produce DAR4 ADCs (FIG. 38).

Similarly experiments were performed for TZB-L128C_F118C, TZB-S134C_F118C, TZB-F170C_S174C, TZB-Q175C_T180C, TZB-S181C_T178C and TZB-S183C_T178C, and the modified antibody all failed to produce DAR4 ADC.

### 12. HC_T139C/C220S, LC_F116C/C214S (Both Arms)

TCEP aqueous solution (2.76 μL, 10 mM in H₂O, 20 equivalents per single antibody molecule) was added to a solution of Trastuzumab (TZB) with a disulfide bond mutation (HC_139C/C220S, LC_116C/C214S) (0.2 mg) in histidine buffer (80 μL, 20 mM, pH=6.5). The obtained mixture was incubated at 37° C. for 2 hours.

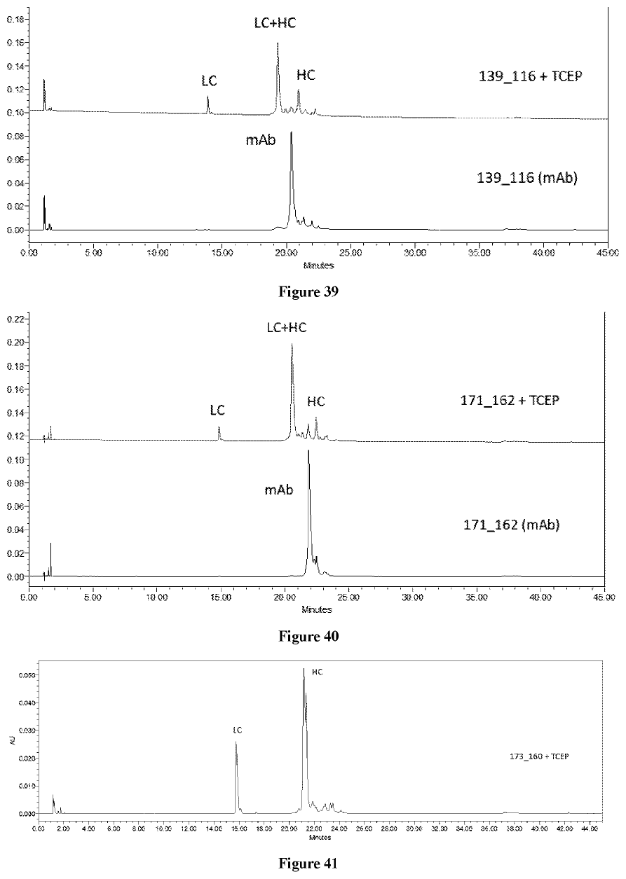

The RP-HPLC analysis of the reduced antibody following the General procedure B indicated that more than 10% of the disulfide bond between the light chain and heavy chain was opened thus the modified antibody failed to produce DAR4 ADCs (FIG. 39).

### 13. HC_P171C/C220S, LC_S162C/C214S (Both Arms)

TCEP aqueous solution (2.76 μL, 10 mM in H₂O, 20 equivalents per single antibody molecule) was added to a solution of Trastuzumab (TZB) with a disulfide bond mutation (HC_171C/C220S, LC_162C/C214S) (0.2 mg) in histidine buffer (80 μL, 20 mM, pH=6.5). The obtained mixture was incubated at 37° C. for 2 hours.

The RP-HPLC analysis of the reduced antibody following the General procedure B indicated that more than 10% of the disulfide bond between the light chain and heavy chain was opened thus the modified antibody failed to produce DAR4 ADCs (FIG. 40).

### 14. HC_V173C/C220S, LC_Q160C/C214S (Both Arms)

TCEP aqueous solution (2.76 μL, 10 mM in H₂O, 20 equivalents per single antibody molecule) was added to a solution of Trastuzumab (TZB) with a disulfide bond mutation (HC_173C/C220S, LC_160C/C214S) (0.2 mg) in histidine buffer (80 μL, 20 mM, pH=6.5). The obtained mixture was incubated at 37° C. for 2 hours.

The RP-HPLC analysis of the reduced antibody following the General procedure B indicated that the disulfide bond between the light chain and heavy chain was opened thus the modified antibody failed to produce DAR4 ADCs (FIG. 41).

### 15. HC_S136C/C220S, LC_S114C/C214S (Both Arms)

TCEP aqueous solution (2.76 μL, 10 mM in H₂O, 20 equivalents per single antibody molecule) was added to a solution of Trastuzumab (TZB) with a disulfide bond mutation (HC_136C/C220S, LC_114C/C214S) (0.2 mg) in histidine buffer (80 μL, 20 mM, pH=6.5). The obtained mixture was incubated at 37° C. for 2 hours.

After cooling to room temperature, a solution of linker-payload (mc-GGFG-Dxd, 1.42 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.8 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

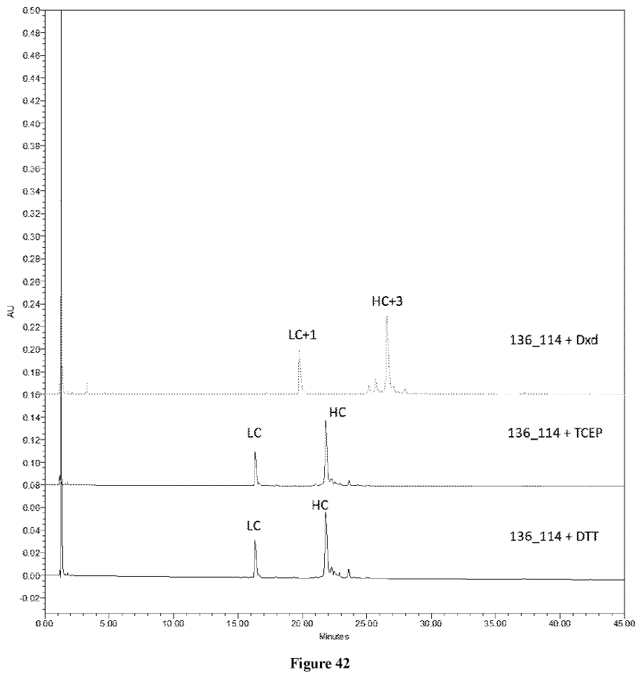

The resulting ADC product was first treated with DTT following the General procedure A, and then analyzed by RP-HPLC. The RP-HPLC analysis following the General procedure B indicated that the modified antibody failed to produce DAR4 ADCs (FIG. 42).

### 16. TZB-S131C_P119C

TCEP aqueous solution (2.76 μL, 5 mM in H₂O, 20 equivalents per single antibody molecule) was added to a solution of an anti-HER2 antibody Trastuzumab (TZB) with disulfide bond mutation at both arms (HC_S131C/C220S, LC_P119C/C214S) (0.1 mg) in histidine buffer (50 μL, 20 mM, pH=6.5). The obtained mixture was incubated at 37° C. for 2 hours, so that the disulfide bonds at hinge region were fully reduced.

After cooling to room temperature, a solution of linker-payload (mc-GGFG-Dxd, 0.71 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.4 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

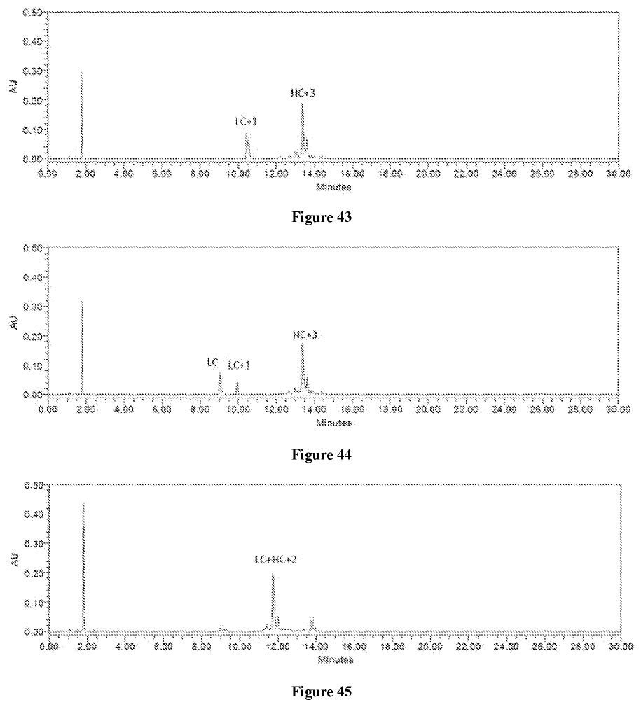

The RP-HPLC analysis following the General procedure B indicated the modified antibody failed to produce DAR4 ADCs (FIG. 43).

### 17. TZB-S132C_F209C

TCEP aqueous solution (2.76 μL, 5 mM in H₂O, 20 equivalents per single antibody molecule) was added to a solution of an anti-HER2 antibody Trastuzumab (TZB) with disulfide bond mutation at both arms (HC_S132C/C220S, LC_F209C/C214S) (0.1 mg) in histidine buffer (50 μL, 20 mM, pH=6.5). The obtained mixture was incubated at 37° C. for 2 hours, so that the disulfide bonds at hinge region were fully reduced.

After cooling to room temperature, a solution of linker-payload (mc-GGFG-Dxd, 0.71 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.4 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

The RP-HPLC analysis following the General procedure B indicated the modified antibody failed to produce DAR4 ADCs (FIG. 44).

### 18. TZB-K133C_I117C (Both Arms)

TCEP aqueous solution (2.76 μL, 5 mM in H₂O, 20 equivalents per single antibody molecule) was added to a solution of an anti-HER2 antibody Trastuzumab (TZB) with disulfide bond mutation at both arms (HC_K133C/C220S, LC_I117C/C214S) (0.1 mg) in histidine buffer (50 μL, 20 mM, pH=6.5). The obtained mixture was incubated at 37° C. for 2 hours, so that the disulfide bonds at hinge region were fully reduced.

After cooling to room temperature, a solution of linker-payload (mc-GGFG-Dxd, 0.71 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.4 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

The RP-HPLC analysis following the General procedure B indicated the drug-to-antibody ratio was 3.77 (FIG. 45).

### 19. Tzb-K133C_K207C

TCEP aqueous solution (2.76 μL, 5 mM in H₂O, 20 equivalents per single antibody molecule) was added to a solution of an anti-HER2 antibody Trastuzumab (TZB) with disulfide bond mutation at both arms (HC_K133C/C220S, LC_K207C/C214S) (0.1 mg) in histidine buffer (50 μL, 20 mM, pH=6.5). The obtained mixture was incubated at 37° C. for 2 hours, so that the disulfide bonds at hinge region were fully reduced.

After cooling to room temperature, a solution of linker-payload (mc-GGFG-Dxd, 0.71 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.4 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

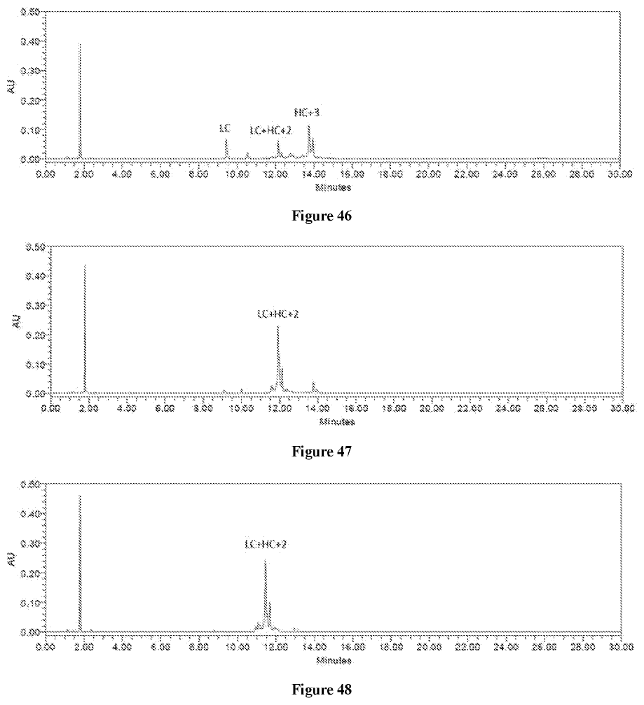

The RP-HPLC analysis following the General procedure B indicated the modified antibody failed to produce DAR4 ADCs (FIG. 46).

### 20. TZB-K133C_F209C (Both Arms)

TCEP aqueous solution (2.76 μL, 5 mM in H₂O, 20 equivalents per single antibody molecule) was added to a solution of an anti-HER2 antibody Trastuzumab (TZB) with disulfide bond mutation at both arms (HC_K133C/C220S, LC_F209C/C214S) (0.1 mg) in histidine buffer (50 μL, 20 mM, pH=6.5). The obtained mixture was incubated at 37° C. for 2 hours, so that the disulfide bonds at hinge region were fully reduced.

After cooling to room temperature, a solution of linker-payload (mc-GGFG-Dxd, 0.71 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.4 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

The RP-HPLC analysis following the General procedure B indicated the drug-to-antibody ratio was 3.80 (FIG. 47).

### 21. TZB-A141C_F116C (Both Arms)

TCEP aqueous solution (2.76 μL, 5 mM in H₂O, 20 equivalents per single antibody molecule) was added to a solution of an anti-HER2 antibody Trastuzumab (TZB) with disulfide bond mutation at both arms (HC_A141C/C220S, LC_F116C/C214S) (0.1 mg) in histidine buffer (50 μL, 20 mM, pH=6.5). The obtained mixture was incubated at 37° C. for 2 hours, so that the disulfide bonds at hinge region were fully reduced.

After cooling to room temperature, a solution of linker-payload (mc-GGFG-Dxd, 0.71 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.4 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

The RP-HPLC analysis following the General procedure B indicated the drug-to-antibody ratio was 3.74 (FIG. 48).

### 22. TZB-K133C_I117C (Both Arms)

TCEP aqueous solution (2.76 μL, 10 mM in H₂O, 20 equivalents per single antibody molecule) was added to a solution of an anti-HER2 antibody Trastuzumab (TZB) with disulfide bond mutation at both arms (HC_K133C/C220S, LC_I117C/C214S) (0.2 mg) in histidine buffer (0.1 mL, 20 mM, pH=6.5). The obtained mixture was incubated at 37° C. for 2 hours, so that the disulfide bonds at hinge region were fully reduced.

After cooling to room temperature, a solution of linker-payload (mc-VC-PAB-MMAE, 1.82 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.8 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

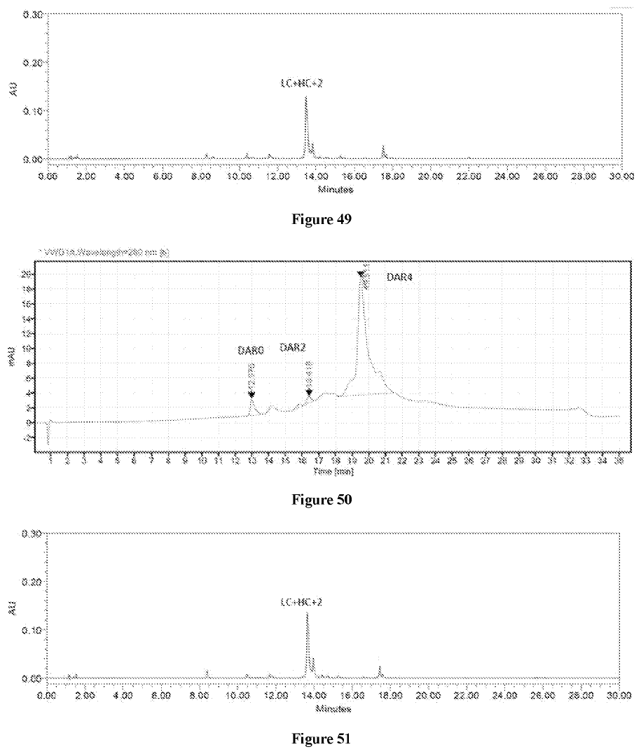

The RP-HPLC analysis following the General procedure B indicated the drug-to-antibody ratio was 3.76 (FIG. 49).

HIC analysis following the General procedure D showed the percentages of ADCs with different amounts of conjugated drugs was as follows: D0, 4.89%; D2, 2.85%; D4, 92.25% (FIG. 50).

### 23. TZB-K133C_F209C (Both Arms)

TCEP aqueous solution (2.76 μL, 10 mM in H₂O, 20 equivalents per single antibody molecule) was added to a solution of an anti-HER2 antibody Trastuzumab (TZB) with disulfide bond mutation at both arms (HC_K133C/C220S, LC_F209C/C214S) (0.2 mg) in histidine buffer (0.1 mL, 20 mM, pH=6.5). The obtained mixture was incubated at 37° C. for 2 hours, so that the disulfide bonds at hinge region were fully reduced.

After cooling to room temperature, a solution of linker-payload (mc-VC-PAB-MMAE, 1.82 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.8 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

The RP-HPLC analysis following the General procedure B indicated the drug-to-antibody ratio was 3.89 (FIG. 51).

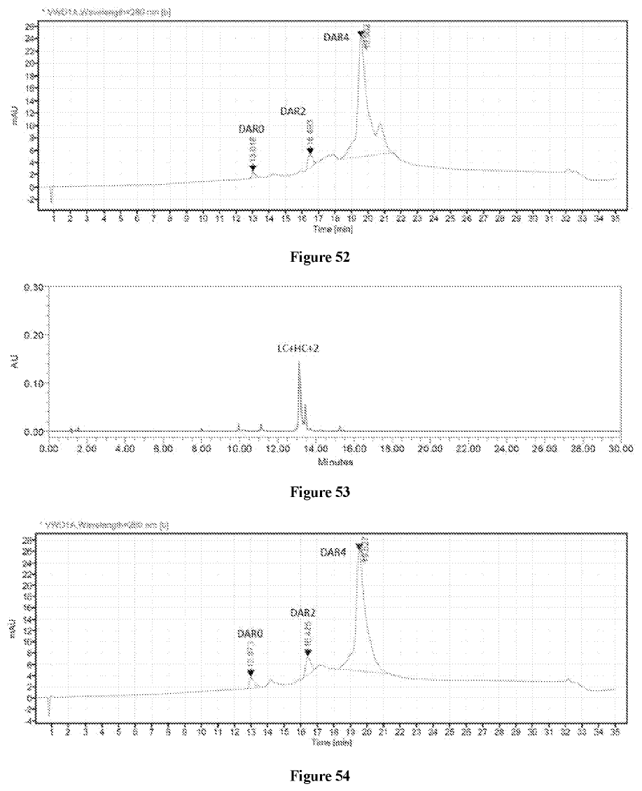

HIC analysis following the General procedure D showed the percentages of ADCs with different amounts of conjugated drugs was as follows: D0, 1.58%; D2, 4.26%; D4, 94.16% (FIG. 52).

### 24. TZB-A141C_F116C (Both Arms)

TCEP aqueous solution (2.76 μL, 10 mM in H₂O, 20 equivalents per single antibody molecule) was added to a solution of an anti-HER2 antibody Trastuzumab (TZB) with disulfide bond mutation at both arms (HC_A141C/C220S, LC_F116C/C214S) (0.2 mg) in histidine buffer (0.1 mL, 20 mM, pH=6.5). The obtained mixture was incubated at 37° C. for 2 hours, so that the disulfide bonds at hinge region were fully reduced.

After cooling to room temperature, a solution of linker-payload (mc-VC-PAB-MMAE, 1.82 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.8 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

The RP-HPLC analysis following the General procedure B indicated the drug-to-antibody ratio was 3.74 (FIG. 53).

HIC analysis following the General procedure D showed the percentages of ADCs with different amounts of conjugated drugs was as follows: D0, 3.59%; D2, 6.55%; D4, 89.77% (FIG. 54)

### 25. TZB-Hel-F126C_Q124C (Single Arm)

TCEP aqueous solution (2.08 μL, 10 mM in H₂O, 15 equivalents per single antibody molecule) was added to a solution of an anti-HER2 bispecific antibody (Trastuzumab-Hel, or TZB-Hel) with disulfide bond mutation at the TZB arms (HC_F126C/C220S, LC_Q124C/C214S) (0.2 mg) in histidine buffer (0.1 mL, 20 mM, pH=6.5). The obtained mixture was incubated at 37° C. for 2 hours, so that the disulfide bonds at hinge region were fully reduced.

After cooling to room temperature, a solution of linker-payload (mc-VC-PAB-MMAE, 2.74 μL, 10 mg/mL in DMSO, 15 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.8 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

The resulting ADC product was first treated with DTT following the General procedure A, and then analyzed by RP-HPLC. The RP-HPLC analysis following the General procedure B indicated the drug-to-antibody ratio was 5.85 (FIG. 55).

HIC analysis following the General procedure D showed the percentages of ADCs with different amounts of conjugated drugs was as follows: D0, 0%; D2, 2.96%; D4, 4.70%; D6, 92.34% (FIG. 56).

### 26. TZB-P130C_F118C (Both Arms)

TCEP aqueous solution (2.76 μL, 10 mM in H₂O, 20 equivalents per single antibody molecule) was added to a solution of an anti-HER2 antibody Trastuzumab (TZB) with disulfide bond mutation at both arms (HC_P130C/C220S, LC_F118C/C214S) (0.2 mg) in histidine buffer (0.1 mL, 20 mM, pH=6.5). The obtained mixture was incubated at 37° C. for 2 hours, so that the disulfide bonds at hinge region were fully reduced.

After cooling to room temperature, a solution of linker-payload (mc-VC-PAB-MMAE, 1.82 μL, 10 mg/mL in DMSO, 10 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.8 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without purification.

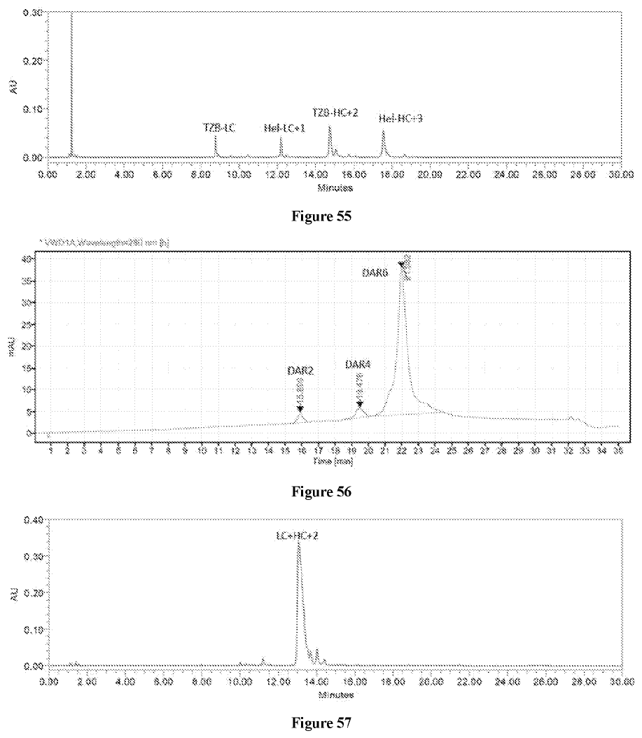

The RP-HPLC analysis following the General procedure B indicated the drug-to-antibody ratio was 3.95 (FIG. 57).

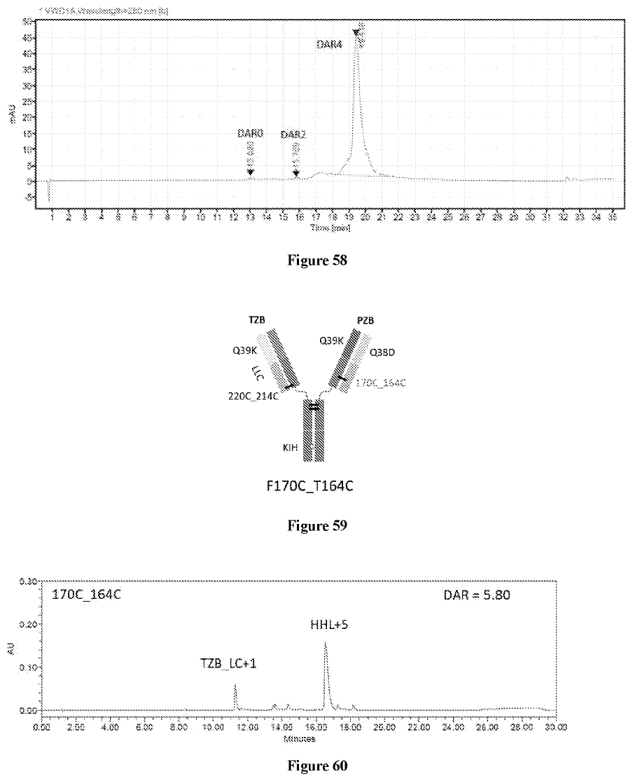

HIC analysis following the General procedure D showed the percentages of ADCs with different amounts of conjugated drugs was as follows: D0, 0.54%; D2, 0.84%; D4, 98.62% (FIG. 58).

### 27. HC_F170C/C220S, LC_T164C/C214S (Single Arm)

TCEP aqueous solution (8.23 μL, 10 mM in H₂O, 12 equivalents per single antibody molecule) was added to a solution of a bispecific antibody (Trastuzumab-Pertuzumab, or TZB-PZB) with a disulfide bond mutation at the PZB arm (HC_F170C/C220S, LC_T164C/C214S) (1.0 mg) in histidine buffer (0.33 mL, 20 mM, pH=6.5). The obtained mixture was incubated at 25° C. for 2 hours.

After cooling to room temperature, a solution of linker-payload (mc-vc-PAB-MMAE, 10.8 μL, 10 mg/mL in DMSO, 12 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (4.2 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (2 mL), and the remaining solution was concentrated to obtain a solution of corresponding ADC (0.83 mg, 16.6 mg/mL, 50 μL).

FIG. 59 is a schematic illustration of the ADC structure.

The RP-HPLC analysis following the General procedure B indicated the drug-to-antibody ratio was 5.80 (FIG. 60).

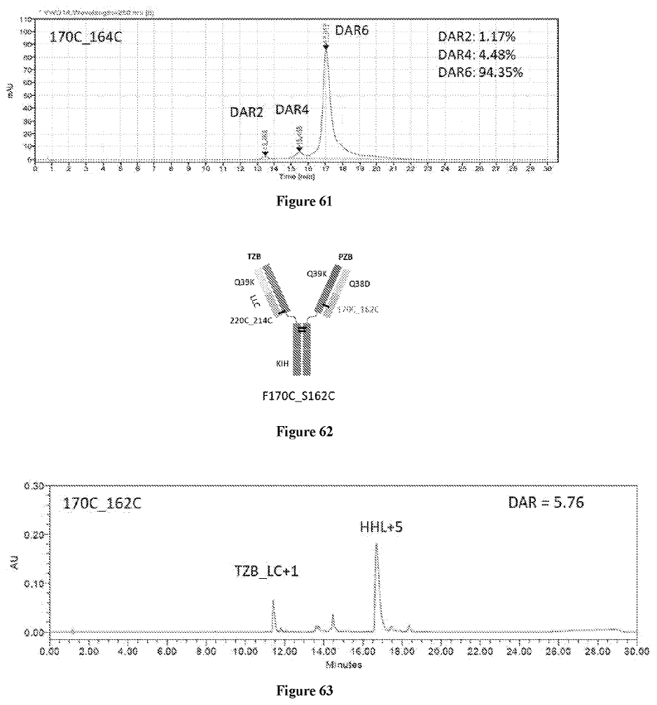

HIC analysis following the General procedure D showed the percentages of ADCs with different amounts of conjugated drugs was as follows: D2, 1.17%; D4, 4.48%; D6, 94.35% (FIG. 61).

### 28. HC_F170C/C220S, LC_S162C/C214S (Single Arm)

TCEP aqueous solution (4.11 μL, 10 mM in H₂O, 12 equivalents per single antibody molecule) was added to a solution of a bispecific antibody (Trastuzumab-Pertuzumab, or TZB-PZB) with a disulfide bond mutation at the PZB arm (HC_F170C/C220S, LC_S162C/C214S) (0.5 mg) in histidine buffer (0.17 mL, 20 mM, pH=6.5). The obtained mixture was incubated at 25° C. for 2 hours.

After cooling to room temperature, a solution of linker-payload (mc-vc-PAB-MMAE, 5.4 μL, 10 mg/mL in DMSO, 12 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (2.1 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (0.5 mL), and the remaining solution was concentrated to obtain a solution of corresponding ADC (0.42 mg, 8.3 mg/mL, 50 μL).

FIG. 62 is a schematic illustration of the ADC structure.

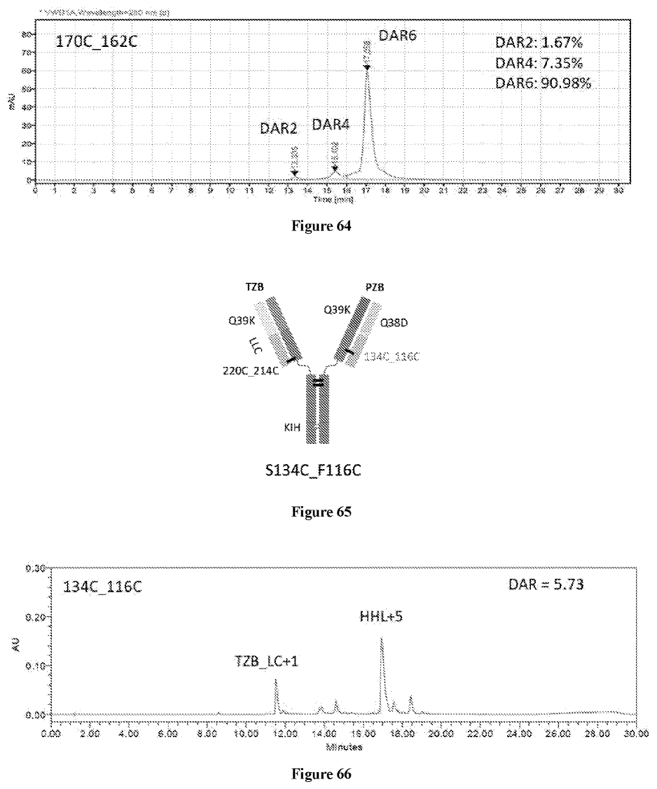

The RP-HPLC analysis following the General procedure B indicated the drug-to-antibody ratio was 5.76 (FIG. 63).

HIC analysis following the General procedure D showed the percentages of ADCs with different amounts of conjugated drugs was as follows: D2, 1.67%; D4, 7.35%; D6, 90.98% (FIG. 64).

### 29. HC_S134C/C220S, LC_F116C/C214S (Single Arm)

TCEP aqueous solution (8.23 μL, 10 mM in H₂O, 12 equivalents per single antibody molecule) was added to a solution of a bispecific antibody (Trastuzumab-Pertuzumab, or TZB-PZB) with a disulfide bond mutation at the PZB arm (HC_S134C/C220S, LC_F116C/C214S) (1.0 mg) in histidine buffer (0.33 mL, 20 mM, pH=6.5). The obtained mixture was incubated at 25° C. for 2 hours.

After cooling to room temperature, a solution of linker-payload (mc-vc-PAB-MMAE, 10.8 μL, 10 mg/mL in DMSO, 12 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (4.2 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (2 mL), and the remaining solution was concentrated to obtain a solution of corresponding ADC (0.82 mg, 16.3 mg/mL, 50 μL).

FIG. 65 is a schematic illustration of the ADC structure.

The RP-HPLC analysis following the General procedure B indicated the drug-to-antibody ratio was 5.73 (FIG. 66).

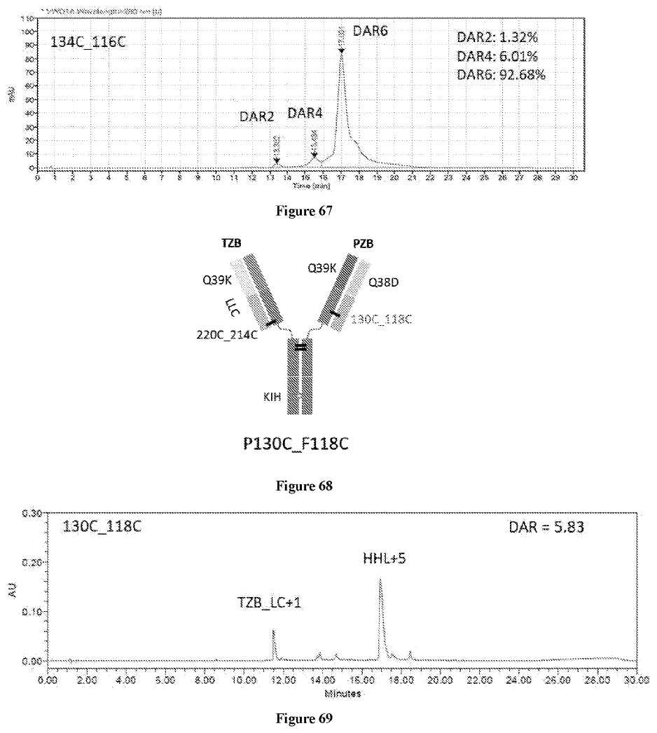

HIC analysis following the General procedure D showed the percentages of ADCs with different amounts of conjugated drugs was as follows: D2, 1.32%; D4, 6.01%; D6, 92.68% (FIG. 67).

### 30. HC_P130C/C220S, LC_F118C/C214S (Single Arm)

TCEP aqueous solution (8.23 μL, 10 mM in H₂O, 12 equivalents per single antibody molecule) was added to a solution of a bispecific antibody (Trastuzumab-Pertuzumab, or TZB-PZB) with a disulfide bond mutation at the PZB arm (HC_P130C/C220S, LC_F118C/C214S) (1.0 mg) in histidine buffer (0.33 mL, 20 mM, pH=6.5). The obtained mixture was incubated at 25° C. for 2 hours.

After cooling to room temperature, a solution of linker-payload (mc-vc-PAB-MMAE, 10.8 μL, 10 mg/mL in DMSO, 12 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (4.2 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (2 mL), and the remaining solution was concentrated to obtain a solution of corresponding ADC (0.78 mg, 15.5 mg/mL, 50 μL).

FIG. 68 is a schematic illustration of the ADC structure.

The RP-HPLC analysis following the General procedure B indicated the drug-to-antibody ratio was 5.83 (FIG. 69).

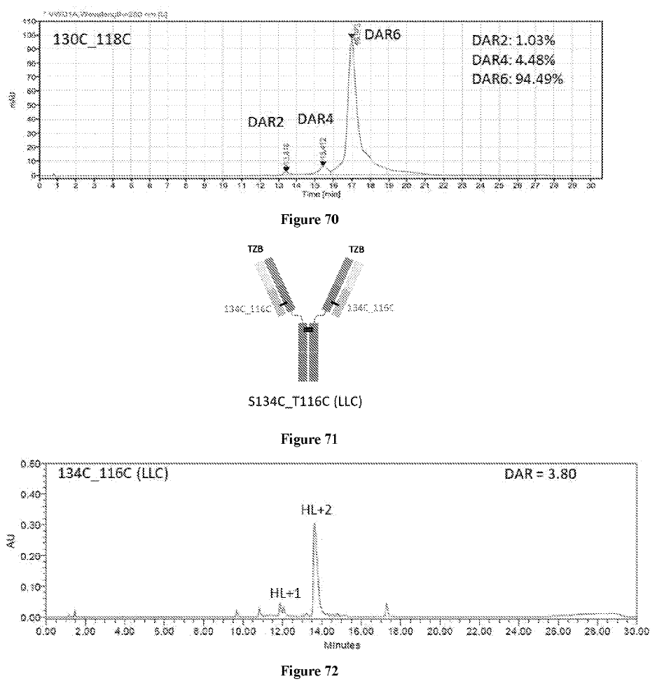

HIC analysis following the General procedure D showed the percentages of ADCs with different amounts of conjugated drugs was as follows: D2, 1.03%; D4, 4.48%; D6, 94.49% (FIG. 70).

### 31. HC_S134C/C220S, LLC_T116C/C214S (Both Arms)

Tris (2-carboxyethyl) phosphine hydrochloride (TCEP) aqueous solution (3.43 μL, 10 mM in H₂O, 10 equivalents per single antibody molecule) was added to a solution of humanized anti-HER2 antibody (trastuzumab (TZB)) with disulfide bond mutations at both arms (HC_S134C/C220S, LLC_T116C/C214S) (0.5 mg) in histidine buffer (0.2 mL, 20 mM, pH=6.5). The obtained mixture was incubated at 25° C. for 2 hours, so that the disulfide bonds at hinge region were fully reduced.

After cooling to room temperature, a solution of linker-payload (mc-vc-PAB-MMAE, 4.5 μL, 10 mg/mL in DMSO, 10 equivalents per single antibody molecule) was added and additional DMSO was added to reach a final concentration of 10% (DMSO/buffer, v/v). The reaction mixture was incubated at 25° C. for 1.5 hours. Subsequently, a 50 mM N-acetylcysteine aqueous solution (2.1 μL, pH=8.0, 30 equivalents per single antibody molecule) was added, and the mixture was stirred for another 20 minutes to quench the unreacted linker-payload. Thereafter, the mixture was desalted by a Zeba™ Spin Desalting Column (0.5 mL), and the remaining solution was concentrated to obtain a solution of corresponding ADC (0.4 mg, 3.3 mg/mL, 0.12 mL).

FIG. 71 is a schematic illustration of the ADC structure.

RP-HPLC analysis following the General procedure B indicated the drug-to-antibody ratio was 3.84 (FIG. 72).

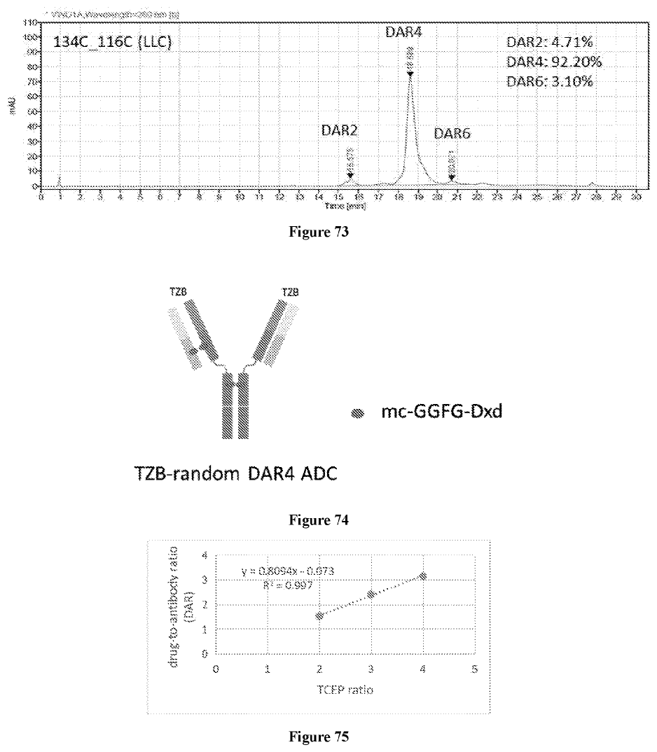

HIC analysis following the General procedure D showed the percentages of ADCs with different amounts of conjugated drugs was as follows: D2, 4.71%; D4, 92.20%; D6, 3.10% (FIG. 73).

### Example 5: Production of Random DAR4 ADCs and Analysis

The purified antibodies were linked to drugs according to the methods below to obtain ADCs.

### a) Pretest to Determine the TCEP Ratio

TCEP aqueous solution (2.76 μL, 1 mM in H₂O, 2 equivalents per single antibody molecule) was added to a solution of an anti-HER2 antibody (trastuzumab) (0.2 mg) in histidine buffer (0.10 mL, 20 mM, pH=6.5). The obtained mixture was incubated at 37° C. for 2 hours.

After cooling to room temperature, a solution of linker-payload (mc-GGFG-Dxd, 1.14 μL, 10 mg/mL in DMSO, 8 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (0.83 μL, pH=8.0, 30 equivalents per single antibody molecule) was added, and the obtained mixture was further stirred at the same temperature for another 20 minutes to quench the unreacted linker-payload. Thereafter, the mixture was subjected to analysis without further

Following the abovementioned procedure, another two TCEP ratios were tested (4.14 μL, 1 mM in H₂O, 3 equivalents per single antibody molecule; 5.52 μL, 1 mM in H₂O, 4 equivalents per single antibody molecule) to provide the corresponding ADC products.

FIG. 74 is a schematic illustration of the ADC structure.

The resulting ADC products were first treated with DTT following the General procedure A, and then analyzed by RP-HPLC. The RP-HPLC analysis following the General procedure B indicated the drug-to-antibody ratios were 1.52, 2.41, 3.14 for 2, 3 and 4 equivalents of TCEP ratios, respectively (FIG. 75).

### b) Prepare Random DAR4 ADC Using the Determined TCEP Ratio

According to the results from the pretest, TCEP ratio of 5.1 should be used to produce the DAR4 ADC.

TCEP aqueous solution (492.4 μL, 1 mM in H₂O, 5.1 equivalents per single antibody molecule), To a solution of an anti-HER2 antibody (trastuzumab) (14.0 mg) in histidine buffer (7.0 mL, 20 mM, pH=6.5). The obtained mixture was then incubated at 37° C. for 2 hours.

After cooling to room temperature, a solution of linker-payload (mc-GGFG-Dxd, 79.9 μL, 10 mg/mL in DMSO, 8 equivalents per single antibody molecule) was added and additional DMSO was added to make a final ratio of 10% (DMSO/buffer, v/v). The reaction mixture was further incubated at 25° C. for 1.5 hours. Subsequently, a 50 mM N-acetylcysteine aqueous solution (58 μL, pH=8.0, 30 equivalents per single antibody molecule) was added, and the obtained mixture was further stirred at the same temperature for another 20 minutes to quench the unreacted linker-payload. Thereafter, the mixture was desalted by Zeba™ Spin Desalting Column (10 mL), and the remaining solution was concentrated to obtain a solution of corresponding ADC (12.7 mg, 2.55 mg/mL, 5.0 mL).

The resulting ADC products (also referred as “aveDAR4” or TZB-Dxd or “TZB-DXd aveDAR4”) were first treated with DTT following the General procedure A, and then analyzed by RP-HPLC. The RP-HPLC analysis following the General procedure B indicated the drug-to-antibody ratios was 4.14 (FIG. 77).

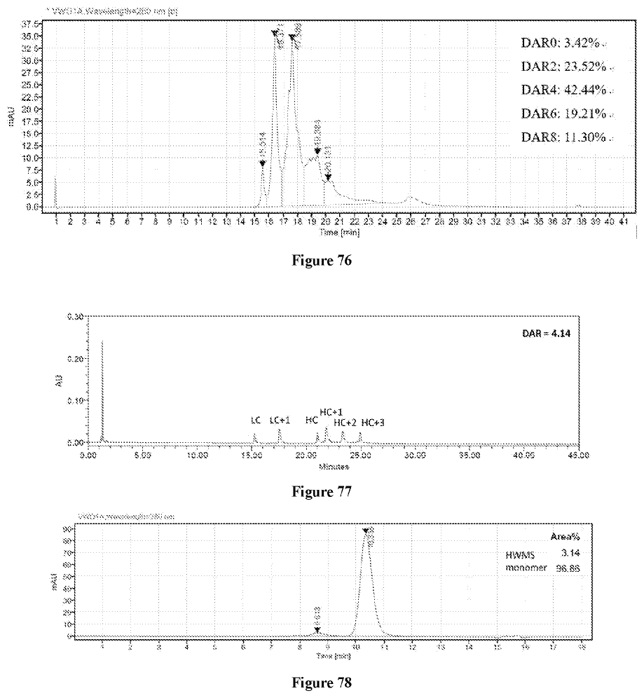

HIC analysis following the General procedure D showed the distribution of the number of conjugated drugs was as follows: D0, 3.42%; D2, 23.52%; D4, 42.44%; D6, 19.31%; D8, 11.30% (FIG. 76).

SEC analysis following the General procedure C indicated the percentages of high-molecular-weight-species (HMWS) and monomer were 3.14%, 96.86%, respectively (FIG. 78).

### c) Preparation of Control IgG1-DXd DAR4

Similar methods were performed to prepare the IgG-Dxd (Control IgG1-DXd DAR4).

FIG. 95 is a schematic illustration of the ADC structure.

The resulting ADC product was first treated with DTT following the General procedure A, and then analyzed by RP-HPLC. The RP-HPLC analysis following the General procedure B indicated the drug-to-antibody ratios was 4.14 (FIG. 96).

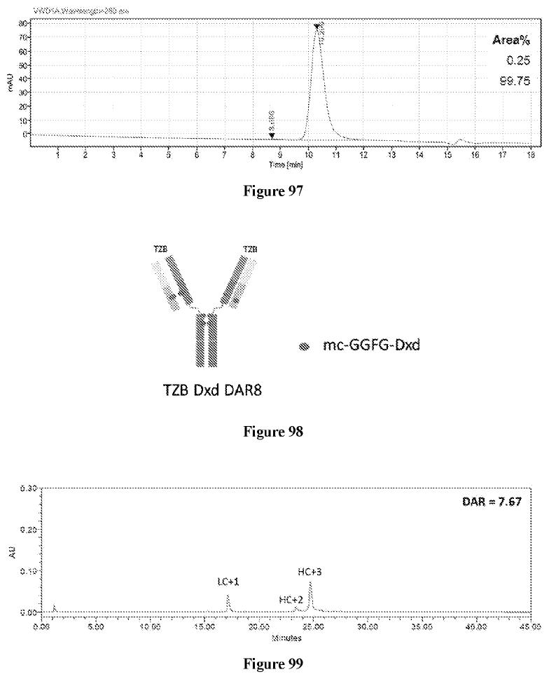

SEC analysis following the General procedure C indicated the percentages of high-molecular-weight-species (HMWS) and monomer were 0.25%, 99.75%, respectively (FIG. 97).

### d) Preparation of TZB-Dxd DAR8

Tris (2-carboxyethyl) phosphine hydrochloride (TCEP) aqueous solution (200 μL, 10 mM in H2O, 20 equivalents per single antibody molecule) was added to a solution of humanized anti-HER2 antibody (trastuzumab (TZB)) (15 mg) in histidine buffer (6.0 mL, 20 mM, pH=6.5). The obtained mixture was incubated at 37° C. for 2 hours.

After cooling to room temperature, a solution of linker-payload (mc-GGFG-Dxd, 139 μL, 10 mg/mL in DMSO, 13 equivalents per single antibody molecule) was added and additional DMSO was added to reach a final concentration of 10% (DMSO/buffer, v/v). The reaction mixture was incubated at 25° C. for 1.5 hours. Subsequently, a 50 mM N-acetylcysteine aqueous solution (63 μL, pH=8.0, 30 equivalents per single antibody molecule) was added, and the mixture was stirred for another 20 minutes to quench the unreacted linker-payload. Thereafter, the mixture was desalted by a Zeba™ Spin Desalting Column (10 mL), and the remaining solution was concentrated to obtain a solution of corresponding ADC (14.3 mg, 3.3 mg/mL, 4.3 mL).

FIG. 98 is a schematic illustration of the ADC structure.

RP-HPLC analysis following the General procedure B indicated the drug-to-antibody ratio was 7.67 (FIG. 99).

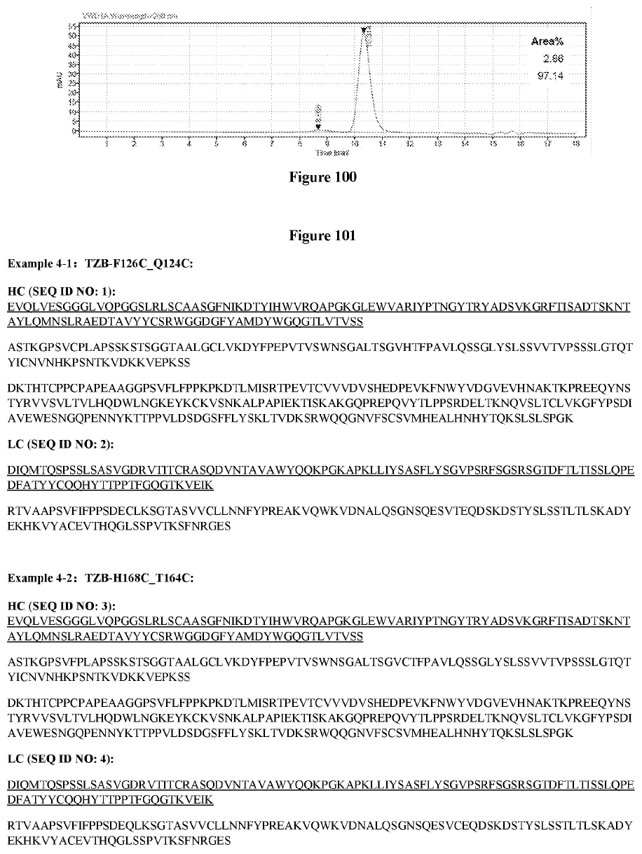

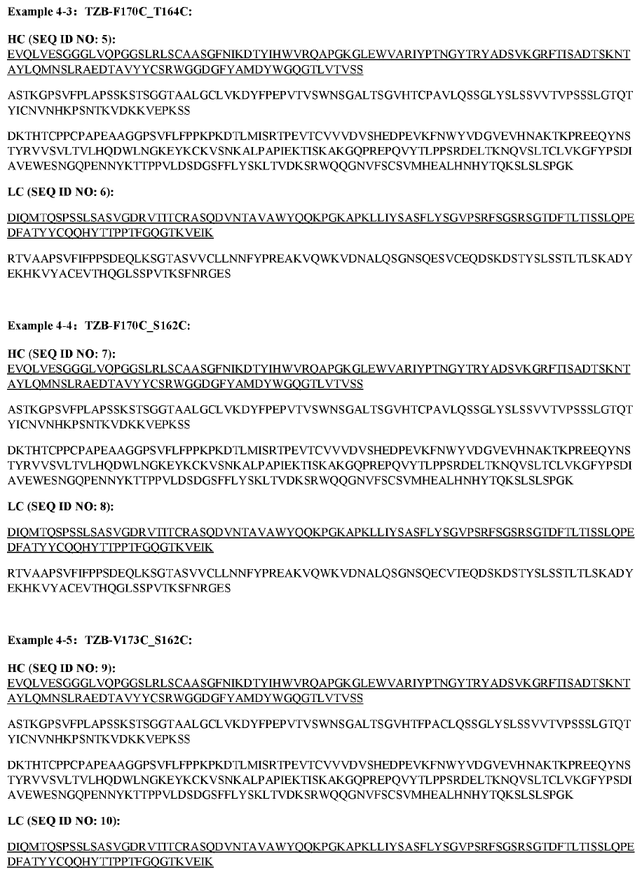

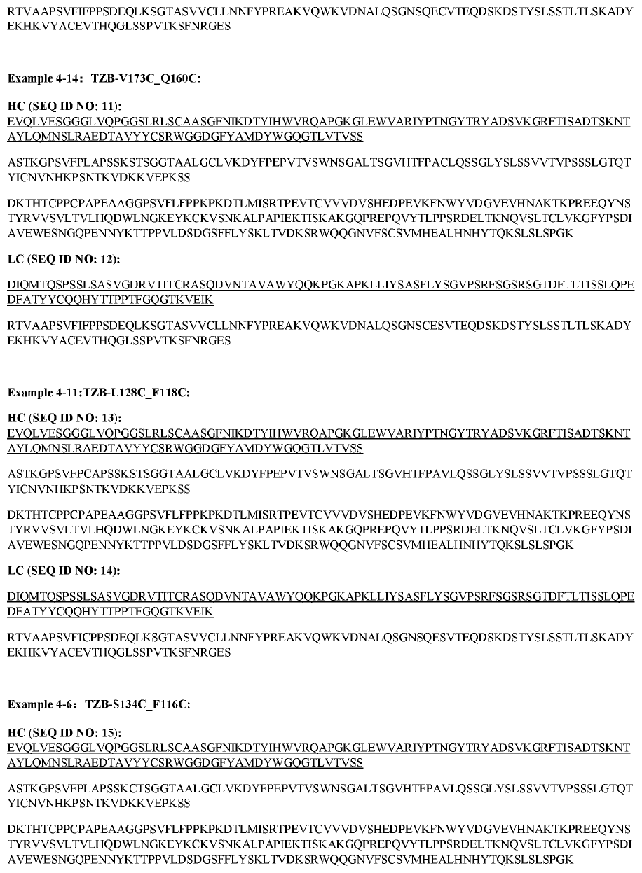

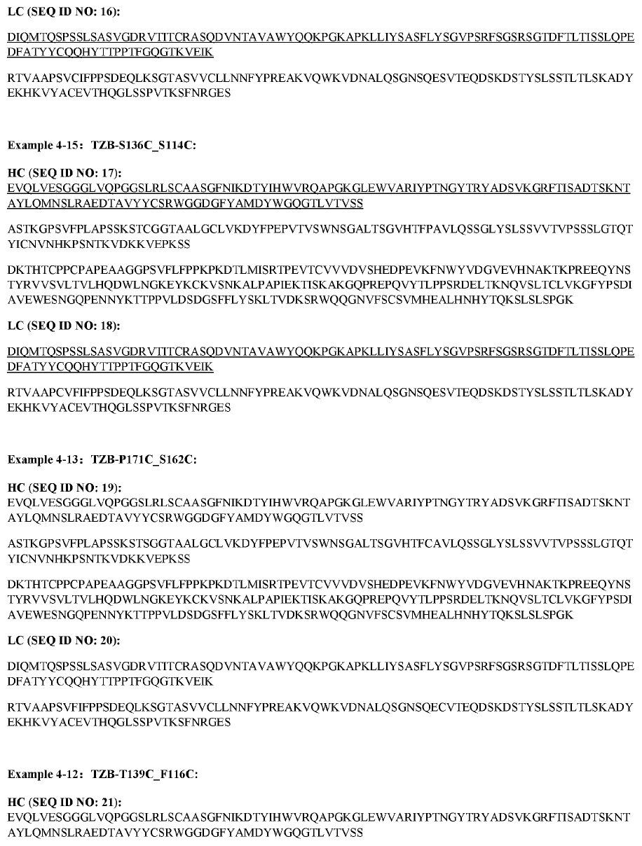

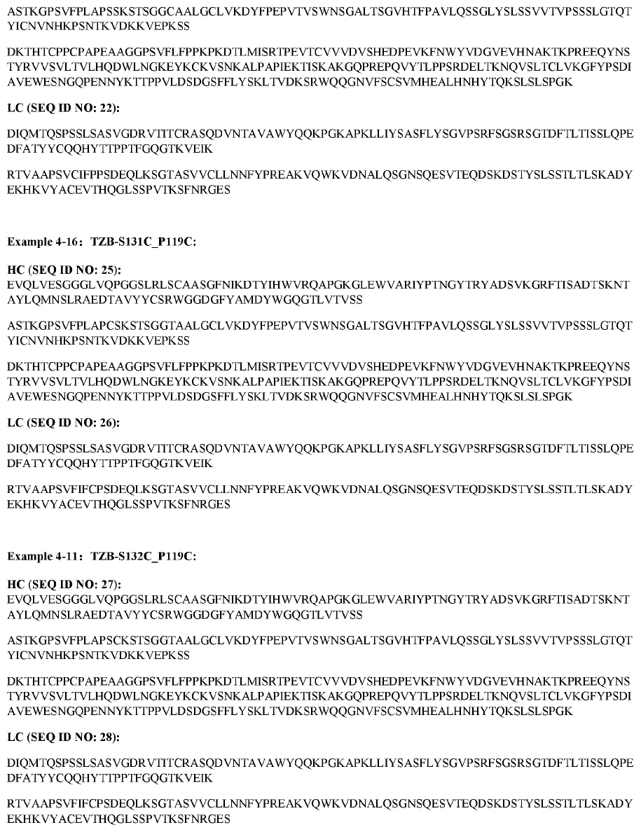

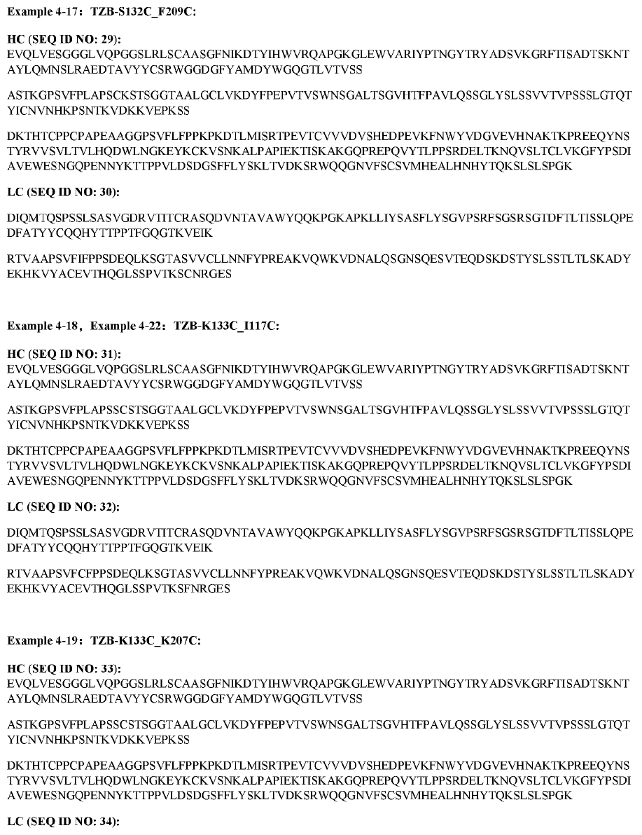

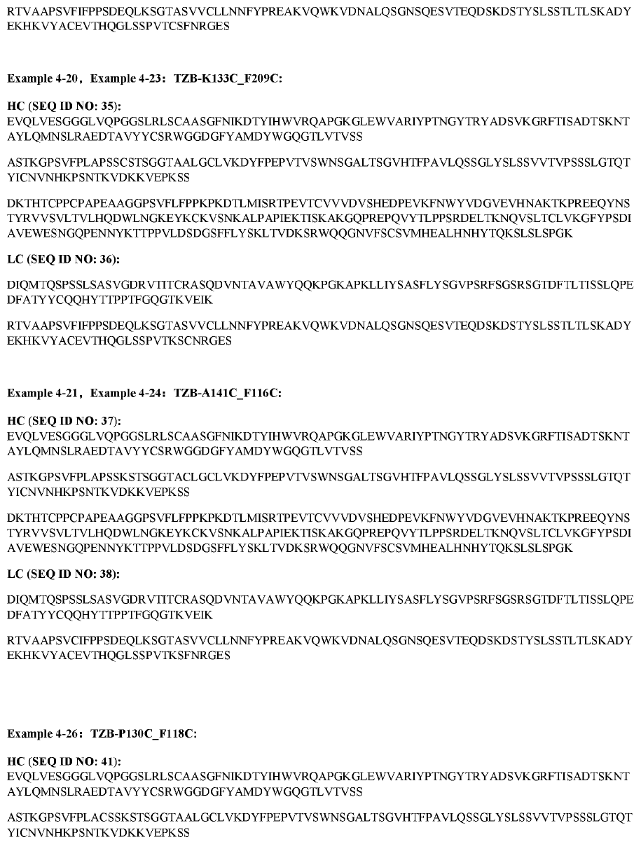

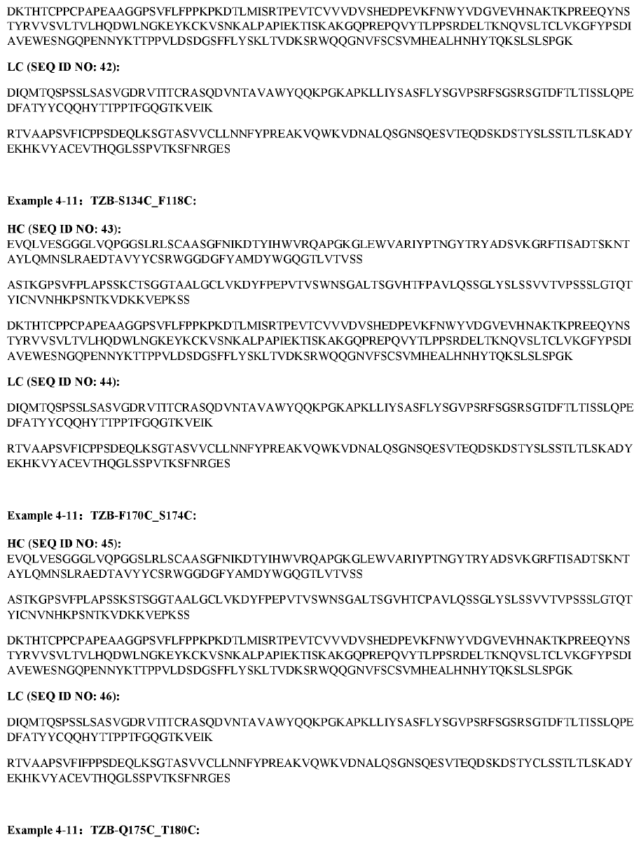

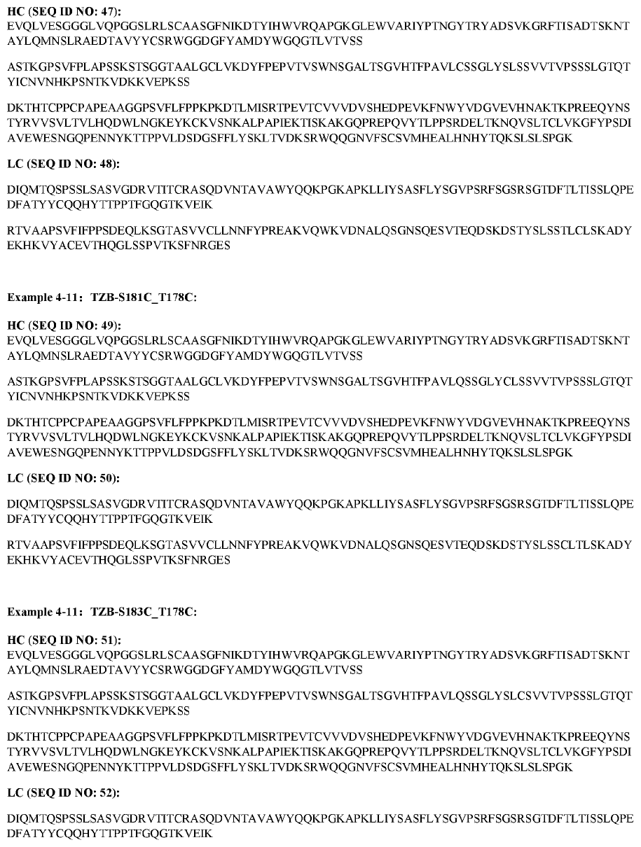

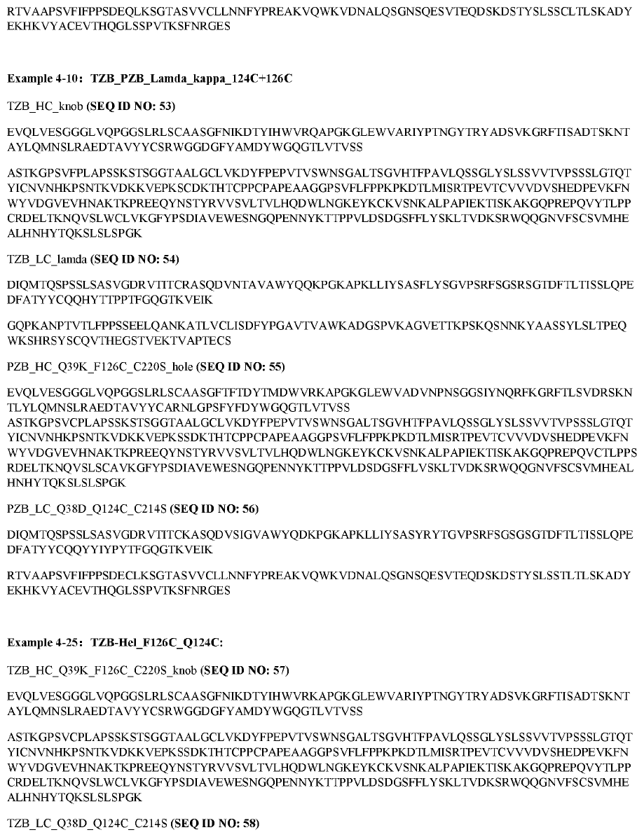

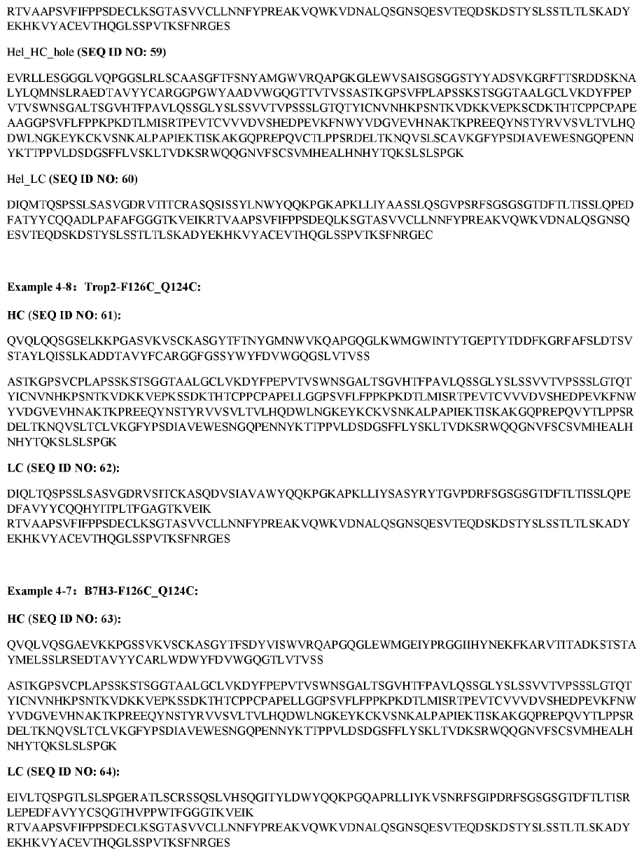

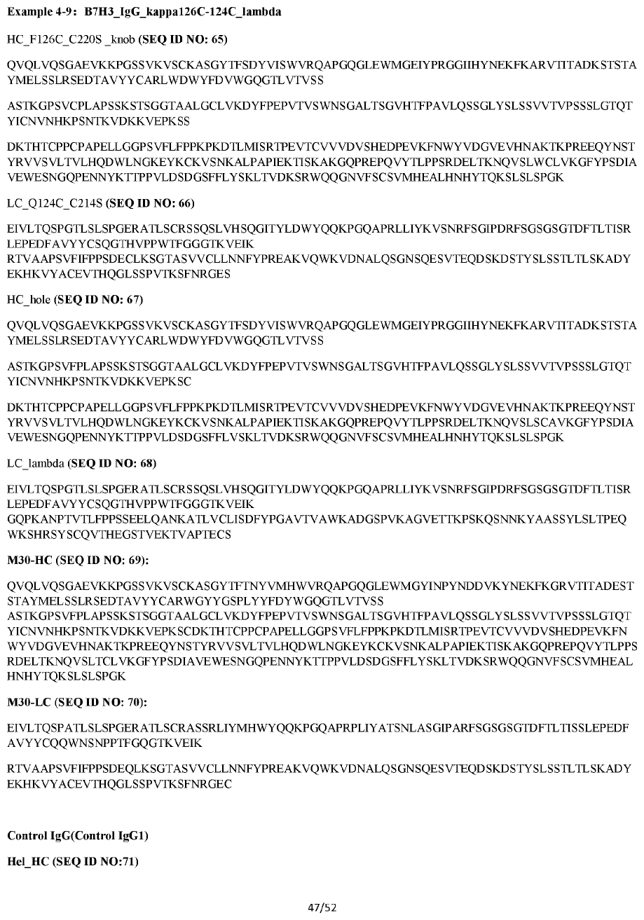

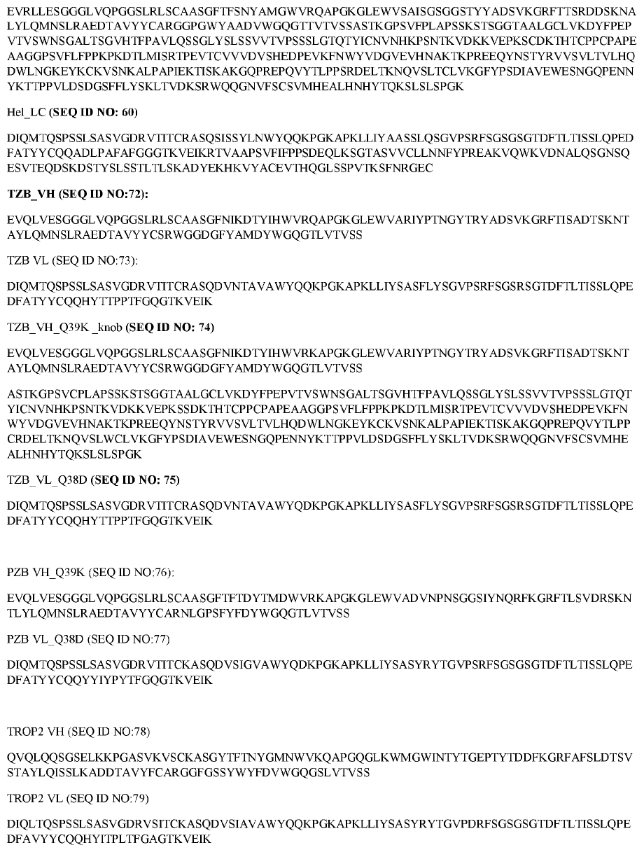

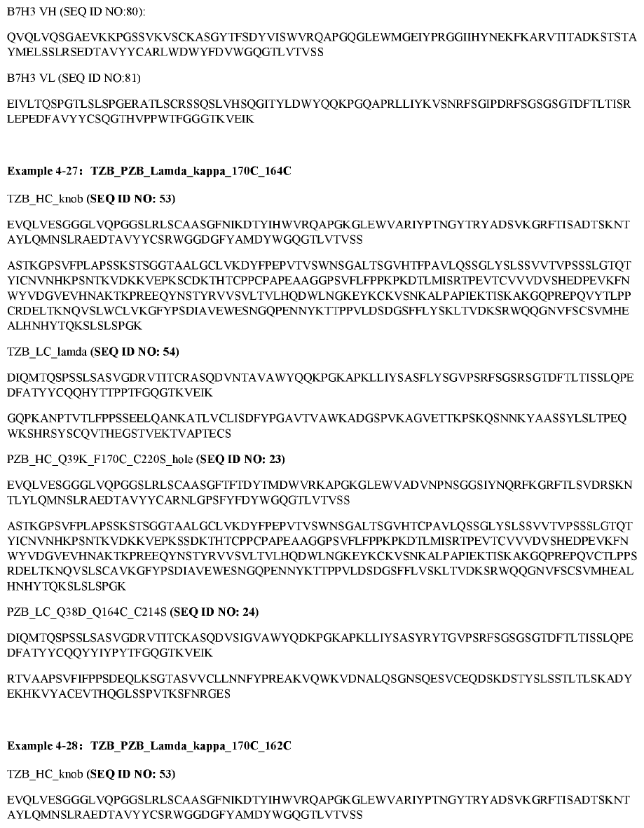

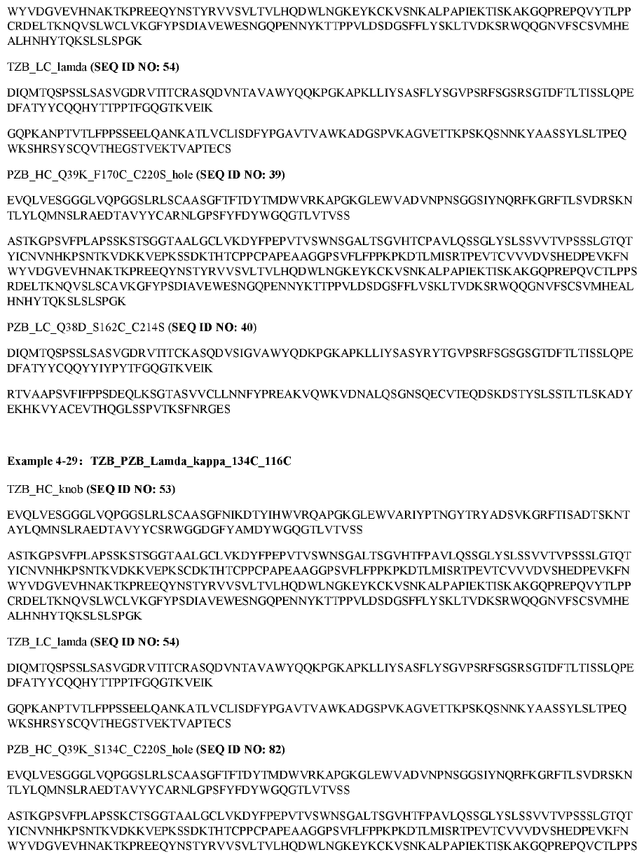

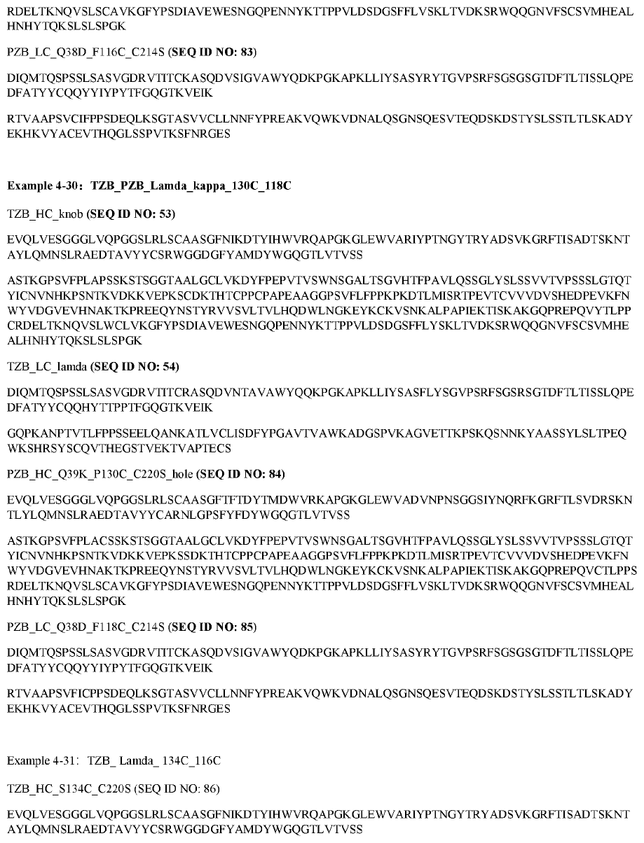

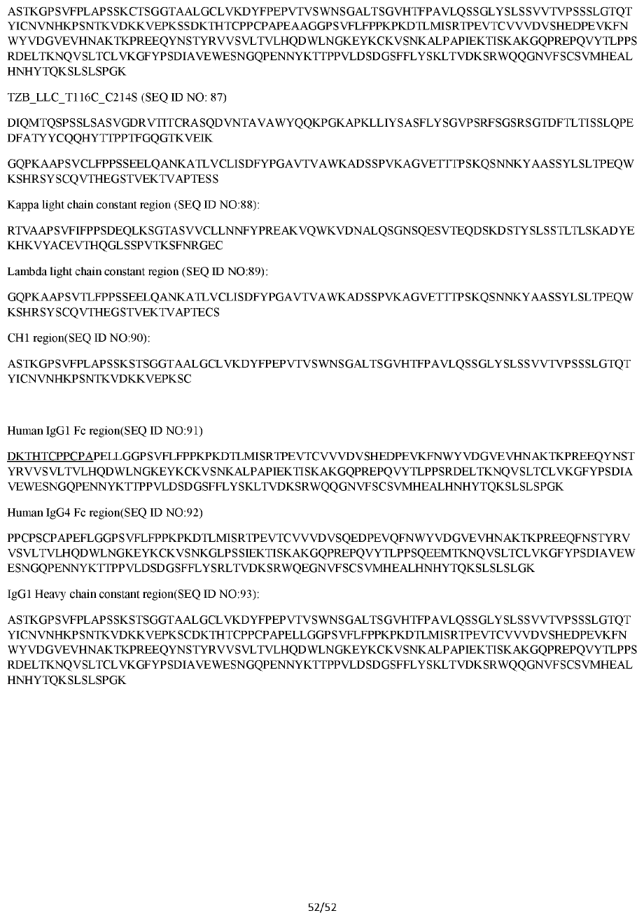

SEC analysis following the General procedure C indicated the percentages of high-molecular-weight-species (HMWS) and monomer were 2.86%, 97.14%, respectively (FIG. 100).

### Example 6: Production of DAR4 ADCs Using Enriching Methods

The purified antibodies were linked to drugs according to the below methods to obtain ADCs.

### a) Pretest to Determine the TCEP Ratio

TCEP aqueous solution (1.025 μL, 5 mM in H₂O, 2.5 equivalents per single antibody molecule) was added to a solution of an anti-B7H3 antibody (M30) (0.3 mg) in histidine buffer (0.06 mL, 20 mM, pH=6.5). The obtained mixture was incubated at 0° C. for 6 hours.

A solution of linker-payload (mc-GGFG-Dxd, 1.7 μL, 10 mg/mL in DMSO, 8 equivalents per single antibody molecule) was added and additional DMSO was added to make a final ratio of 15% (DMSO/buffer, v/v). The reaction mixture was further incubated at 0° C. for 1.0 hour. Subsequently, a 50 mM N-acetylcysteine aqueous solution (1.23 μL, pH=8.0, 30 equivalents per single antibody molecule) was added, and the obtained mixture was further stirred at 25° C. for another 20 minutes to quench the unreacted linker-payload. Thereafter, the mixture was subjected to analysis without further purification.

Following the above-mentioned procedure, another two TCEP ratios were tested (1.435 μL, 5 mM in H₂O, 3.5 equivalents per single antibody molecule; 1.845 μL, 5 mM in H₂O, 4.5 equivalents per single antibody molecule) to provide the corresponding ADC products.

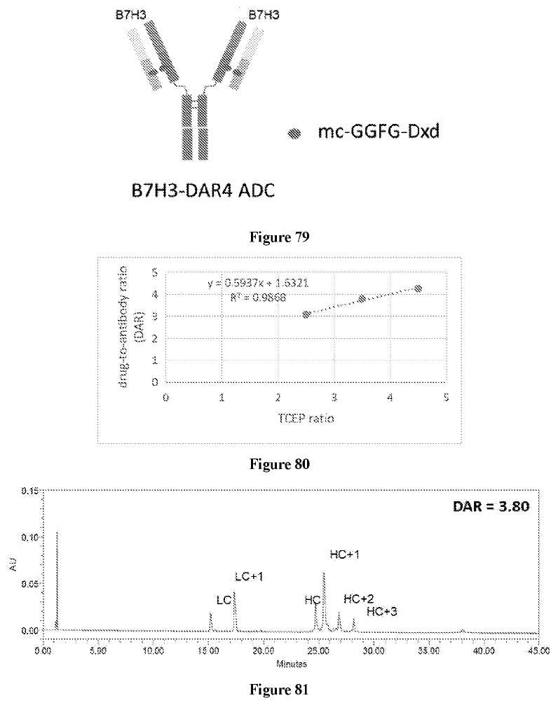

FIG. 79 is a schematic illustration of the ADC structure.

The resulting ADC products were first treated with DTT following the General procedure A, and then analyzed by RP-HPLC. The RP-HPLC analysis following the General procedure B indicated the drug-to-antibody ratios were 3.08, 3.79, 4.26 for 2.5, 3.5 and 4.5 equivalents of TCEP ratio, respectively (FIG. 80).

### b) Prepare Random DAR4 ADC Using the Determined TCEP Ratio

According to the results from the pretest, a TCEP ratio of 4.0 should be used to produce the DAR4 ADC.

TCEP aqueous solution (43.73 μL, 5 mM in H₂O, 4.0 equivalents per single antibody molecule) was added to a solution of an anti-B7H3 antibody (M30) (8.0 mg) in histidine buffer (1.6 mL, 20 mM, pH=6.5). The obtained mixture was incubated at 0° C. for 6 hours.

A solution of linker-payload (mc-GGFG-Dxd, 45.2 μL, 10 mg/mL in DMSO, 8 equivalents per single antibody molecule) was added and additional DMSO was added to make a final ratio of 15% (DMSO/buffer, v/v). The reaction mixture was further incubated at 0° C. for 1.5 hours. Subsequently, a 50 mM N-acetylcysteine aqueous solution (32.8 μL, pH=8.0, 30 equivalents per single antibody molecule) was added, and the obtained mixture was further stirred at 25° C. for another 20 minutes to quench the unreacted linker-payload. Thereafter, the mixture was desalted by Zeba™ Spin Desalting Column (10 mL), and the remaining solution was concentrated to obtain a solution of corresponding ADC (7.2 mg, 2.25 mg/mL, 3.2 mL).

The resulting ADC products-were first treated with DTT following the General procedure A, and then analyzed by RP-HPLC. The RP-HPLC analysis following the General procedure B indicated the drug-to-antibody ratios was 3.80 (FIG. 81).

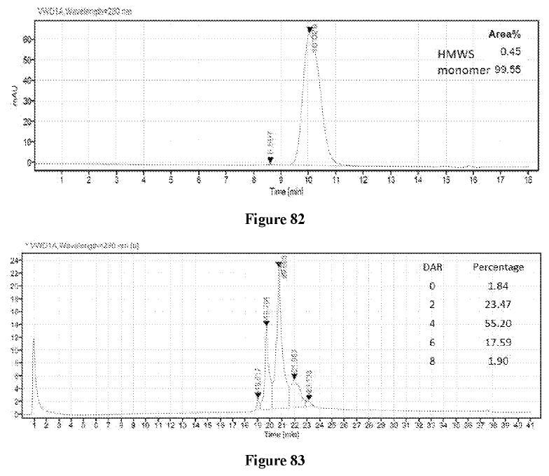

SEC analysis following the General procedure C indicated the percentages of high-molecular-weight-species (HMWS) and monomer were 0.45%, 99.55%, respectively (FIG. 82).

HIC analysis following the General procedure D showed the percentages of ADCs with different amounts of conjugated drugs was as follows: D0, 1.84%; D2, 23.47%; D4, 55.20%; D6, 17.59%; D8, 1.90% (FIG. 83).

### Example 7: Stability of ADCs in Mouse Plasma

The stability of ADCs in mouse plasma was tested following the steps shown below.

Each ADC sample (2 mg/ml) was added in mouse plasma to achieve a final concentration of 0.2 mg/mL (20 μL ADC+180 μL plasma, n=5). The ADC-plasma mixture was incubated at 37° C. One sample was taken at each of the following time points: 0, 1, 3, 5, and 7 days, and stored at less than −40° C. until analysis.

Total human antibody was recovered from plasma by immunoprecipitation using Fc-specific anti-human IgG-agarose resin (Sigma, #A3316). 40 μL Resin slurry was centrifuged to remove the solvent. The resin was rinsed twice with 200 μL PBS (pH 7.4). ADC-containing plasma samples were then gently mixed with the resin for 30 min at room temperature. Resin was recovered by centrifugation, washed twice with PBS of pH 7.4, and washed with PBS of pH 6.0. The resin pellet was re-suspended in 50 μL citrate buffer of pH 3.0, and further incubated for 5-10 min at room temperature. Resin was removed by centrifugation. The supernatant was collected, and adjusted to pH 8.0 with 1 M Tris.

The ADC concentrations were measured by nanodrop.

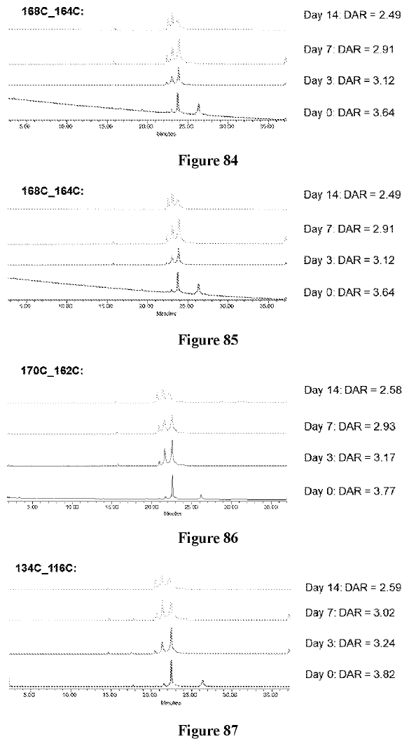

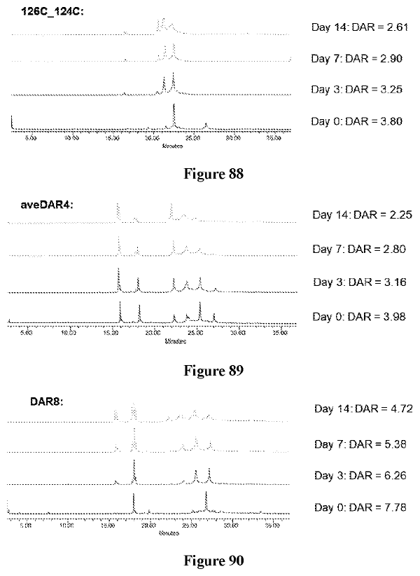

The samples were reduced with 20 equivalents of TCEP, then analyzed by RP-HPLC following the General procedure B. The results are shown in FIGS. 84-90.

### Example 8: Cell Binding Assays

SK-BR-3 cells (ATCC, HTB-30) were re-suspended in FACS buffer (2 mL, 2% FBS, 1× PBS) and adjusted to 1.5~2.0×10⁶/mL, planted into 96-well plates (50 μL per well), and incubated at 4° C. until use.

Trastuzumab and various ADC samples were diluted with FACS buffer to 180 μg/mL, then serial-diluted with the same buffer (4-fold dilutions). The resulting solutions were added to the plates (50 μL per well) and the plates were incubated at 4° C. for 30 min. After centrifugation, the supernatant was discarded and the cells were washed with FACS buffer. Next, PE-goat anti-human IgG (purchased from Biolegend) was added, then the cells were re-suspended and incubated in dark at 4° C. for 30 min. The cells were centrifuged, washed with FACS buffer, and analyzed by a flow cytometer.

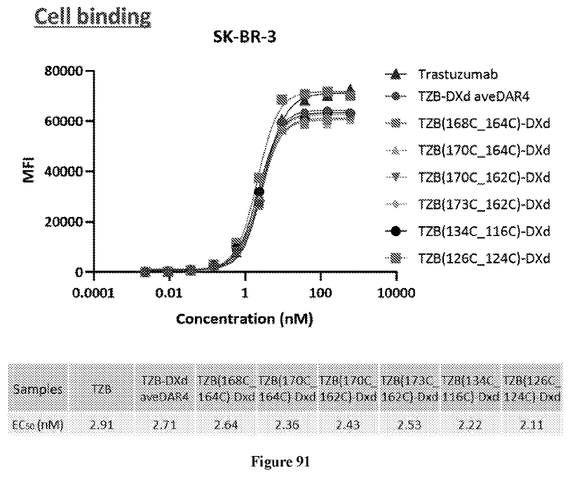

The results are shown in FIG. 91.

### Example 9: In Vitro Cytotoxicity Assays

SK-BR-3 cells were planted into 96-well plates at 2,000 cells per well. These plates were incubated overnight at 37° C. with 5% CO₂.

Serial 3-fold dilutions were applied to the ADC samples with the corresponding medium, starting from 40 nM. The ADC samples were added to the cells (two wells at each concentration gradient) and the cells were cultured at 37° C. and 5% CO₂ for 6 days before the addition of CellTiter-Glo® Luminescent (100 μL per well). The plates were gently shaken for 2 min and incubated for 10 min before measurement at 450 nm using a spectrophotometer. The EC50 values and the cell viability curve were calculated by GraphPad Prism software.

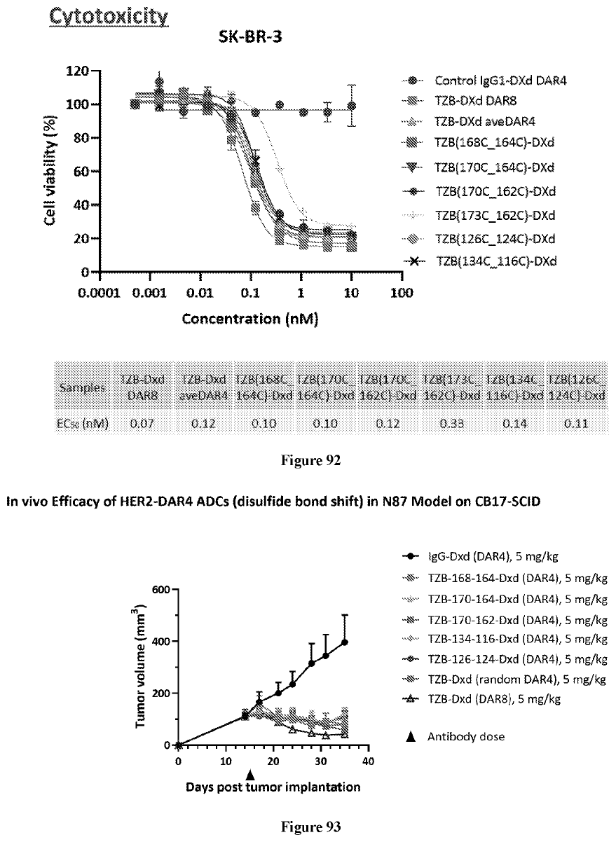

The results are shown in FIG. 92.

### Example 10: In vivo efficacy studies in NCI-N87 Model on CB17-SCID mice

4×10⁶ Human gastric carcinoma cells (NCI-N87, purchased from ATCC) were suspended in a normal saline. Thereafter, the obtained solution was subcutaneously implanted into the CB17-SCID mice (Charles river, Beijing) on Day 0. The mice were randomly divided into groups on Day 11.

After completion of the grouping, the anti-HER2 ADC produced using the enriching method (TZB-Dxd, Example 5) and the site-specific ADCs produced according to Example 4 were each administered to the mice intravenously (i.v.), at single dose of 5 mg/kg. The tumor size and weight were measured every 3 days. Tumor growth inhibition was monitored every three days.

Tumor inhibition (TG1%) was calculated as follows: TGI %=100%*(control group tumor volume-treatment group tumor volume)/(control group tumor volume-initial tumor volume of the control group).

Tumor volume measurement: the maximum long axis (L) and maximum wide axis (W) of the tumor were measured using a vernier caliper. The tumor volume was calculated using the following formula: V=L*W²/2

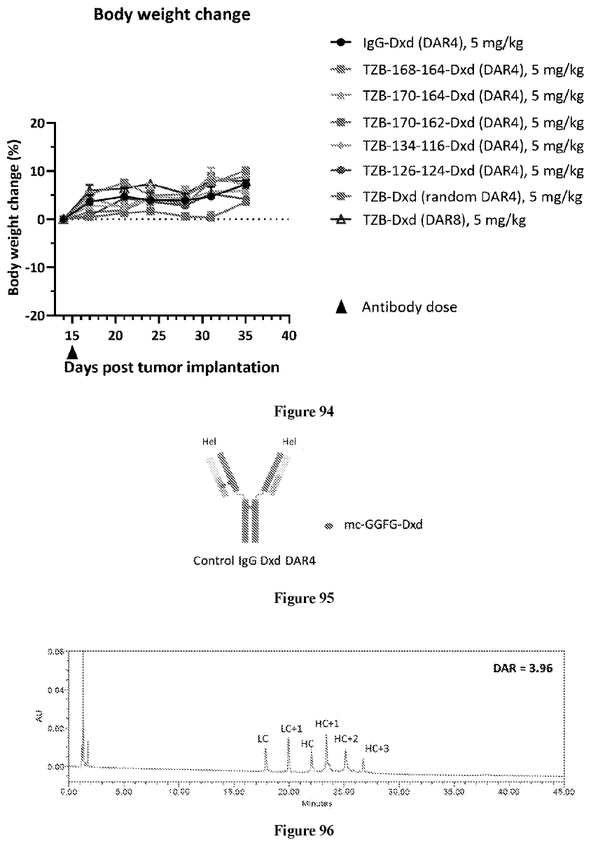

The results are shown in FIG. 93 and FIG. 94 and summarized in the table below.

TABLE 5
Efficacy of DAR4 HER2-ADCs in
NCI-N87 model on CB17-SCID mice

| | 35 Days post | |
|---|---|---|
| | tumor implantation | |
| | Tumor | |
| Group | volume (mm³) | TGI (%) |
| IgG-Dxd, 5 mg/kg | 396.9 | N/A |
| TZB-168-164-Dxd, 5 mg/kg | 80.4 | 111 |
| TZB-170-164-Dxd, 5 mg/kg | 101.0 | 103 |
| TZB-170-162-Dxd, 5 mg/kg | 58.5 | 118 |
| TZB-134-116-Dxd, 5 mg/kg | 119.8 | 97 |
| TZB-126-124-Dxd, 5 mg/kg | 109.4 | 100 |
| TZB-Dxd (random DAR4), 5 mg/kg | 79.6 | 111 |
| TZB-Dxd (DAR8), 5 mg/kg | 41.9 | 124 |

It is demonstrated that the ADC composition produced by the production method of the present invention has therapeutic efficacy equivalent to that of the ADC composition produced by the conventional production method (random DAR4).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody-drug conjugate (ADC) comprising an antibody or antigen-binding fragment thereof, or a pharmaceutically acceptable salt or solvate thereof, wherein the antibody or antigen-binding fragment thereof comprises a heavy chain constant region or CH1 region thereof and a light chain constant region, and comprises a non-native disulfide bond that is formed at the positions of a heavy chain amino acid residue in the heavy chain constant region or CH1 region thereof and a light chain amino acid residue in the light chain constant region, wherein the sum of (1) the relative solvent accessible surface area (SASA) of the heavy chain amino acid residue side chain and (2) the relative SASA of the light chain amino acid residue side chain is below 65%, 60% or 55%, wherein the antibody or antigen-binding fragment thereof does not have a disulfide bond between the amino acid residue at position 220 of the heavy chain constant region and the amino acid residue at position 214 of the light chain constant region, wherein the amino acid position is according to EU numbering.

2. The ADC or a pharmaceutically acceptable salt or solvate thereof of claim 1,
wherein the relative SASA of the heavy chain amino acid side chain is above 30% and the relative SASA of the light chain amino acid side chain is below 15%; or the relative SASA of the light chain amino acid side chain is above 30% and the relative SASA of the heavy chain amino acid side chain is below 15%,
preferably, the relative SASA of the heavy chain amino acid side chain is above 40% and the relative SASA of the light chain amino acid side chain is below 10%; or the relative SASA of the light chain amino acid side chain is above 40% and the relative SASA of the heavy chain amino acid side chain is below 10%,
more preferably, the relative SASA of each of the heavy chain and light chain amino acid side chains is below 20%, 25%, 30%, 35%, 40%, or 45%;
wherein the position of the heavy chain constant region is determined according to the amino acid position of the IgG1 heavy chain numbering, and the position of the light chain constant region is determined according to the amino acid position of the Kappa light chain numbering or Lambda light chain numbering;
wherein the amino acid position is according to EU numbering.

3. The ADC or a pharmaceutically acceptable salt or solvate thereof of claim 1 or 2, wherein the non-native disulfide bond is resistant to tris(2-carboxyethyl) phosphine hydrochloride (TCEP) reduction and/or dithiothreitol (DTT) reduction, for example,
more than 90% or 95% of the non-native disulfide bonds remain intact when the antibody or antigen-binding fragment thereof is reduced by 10-30 equivalents of TCEP per single antibody molecule, wherein for example,
the concentration of the antibody or antigen-binding fragment thereof is between 0.1-50 mg/ml during the reduction reaction;
optionally,
the non-native disulfide bond remains stable under reducing conditions that can reduce more than 90% or 95% of hinge region disulfide bonds of the antibody or antigen-binding fragment, for example, more than 90% or 95% of non-native disulfide bond remains intact.

4. The ADC or a pharmaceutically acceptable salt or solvate thereof of any one claims 1-3, wherein the cysteine at position 220 of the heavy chain constant region and the cysteine at position 214 of the light chain constant region of the antibody or antigen-binding fragment thereof are substituted with non-cysteines, for example, the antibody or antigen-binding fragment thereof contains C220S/A/V in the heavy chain constant region, and C214S/A/V in the light chain constant region.

5. The ADC or a pharmaceutically acceptable salt or solvate thereof of anyone of claims 1-4, wherein a heavy chain amino acid residue of a heavy chain constant region or CH1 thereof, and a light chain amino acid residue of a light chain constant region are mutated to a cysteine residue, thus forming a non-natural disulfide bond.

6. The ADC or a pharmaceutically acceptable salt or solvate thereof of claim 5, wherein the mutated heavy chain amino acid residue is located at one or more positions of the heavy chain constant region selected from the group consisting of: 126, 168, 170, 173, 134, 133, 130, and 141, wherein the amino acid position is according to EU numbering; and/or the mutated light chain amino acid residue is located at one or more positions of the light chain constant region selected from the group consisting of: 124, 164, 162, 116, 209, 117, and 118, wherein the position of the heavy chain constant region is determined according to the amino acid position of the IgG1 heavy chain numbering, and the position of the light chain constant region is determined according to the amino acid position of the Kappa light chain numbering or Lambda light chain numbering.

7. The ADC or a pharmaceutically acceptable salt or solvate thereof of claim 6, wherein the antibody or antigen-binding fragment thereof comprises one or more of the following combinations of mutations:
126C in the heavy chain and 124C in the light chain;
168C in the heavy chain and 164C in the light chain;
170C in the heavy chain and 164C in the light chain;
170C in the heavy chain and 162C in the light chain;
173C in the heavy chain and 162C in the light chain;
134C in the heavy chain and 116C in the light chain;
133C in the heavy chain and 209C in the light chain;
133C in the heavy chain and 117C in the light chain;
130C in the heavy chain and 118C in the light chain; or
141C in the heavy chain and 116C in the light chain.

8. The ADC or a pharmaceutically acceptable salt or solvate thereof of claim 7, wherein the antibody or antigen-binding fragment thereof comprises two pairs of heavy and light chains, of which only one pair of heavy and light chains comprise one of the following combinations of mutations:
F126C in the heavy chain and Q124C in the light chain;
H168C in the heavy chain and T164C in the light chain;
F170C in the heavy chain and T164C in the light chain;
F170C in the heavy chain and S162C in the light chain;
V173C in the heavy chain and S162C in the light chain;
S134C in the heavy chain and F116C in the light chain;
K133C in the heavy chain and F209C in the light chain;
K133C in the heavy chain and I117C in the light chain;
P130C in the heavy chain and F118C in the light chain; and
A141C in the heavy chain and F116C in the light chain;
optionally, the heavy and light chains further comprise C220S in the heavy chain and C214S in the light chain.

9. The ADC or a pharmaceutically acceptable salt or solvate thereof of claim 6, wherein the antibody or antigen-binding fragment thereof comprises two pairs of heavy and light chains, of which each pair of heavy and light chains comprise one or more of the following combinations of mutations:
F126C in the heavy chain and Q124C in the light chain;
H168C in the heavy chain and T164C in the light chain;
F170C in the heavy chain and T164C in the light chain;
F170C in the heavy chain and S162C in the light chain;
V173C in the heavy chain and S162C in the light chain;
S134C in the heavy chain and F116C in the light chain;
K133C in the heavy chain and F209C in the light chain;
K133C in the heavy chain and I117C in the light chain;
P130C in the heavy chain and F118C in the light chain; and
A141C in the heavy chain and F116C in the light chain;
optionally, the heavy and light chains further comprise C220S in the heavy chain and C214S in the light chain.

10. The ADC or a pharmaceutically acceptable salt or solvate thereof of claim 9, wherein the antibody or antigen-binding fragment thereof comprises two pairs of heavy and light chains, wherein
each heavy chain comprises F126C and each light chain comprises Q124C;
each heavy chain comprises H168C and each light chain comprises T164C;
each heavy chain comprises F170C and each light chain comprises T164C;
each heavy chain comprises F170C and each light chain comprises S162C;
each heavy chain comprises V173C and each light chain comprises S162C;
each heavy chain comprises S134C and each light chain comprises F116C;
each heavy chain comprises K133C and each light chain comprises F209C;
each heavy chain comprises K133C and each light chain comprises I117C;
each heavy chain comprises P130C and each light chain comprises F118C; or
each heavy chain comprises A141C and each light chain comprises F116C;
optionally, each heavy chain further comprises C220S and each light chain further comprises C214S.

11. The ADC or a pharmaceutically acceptable salt or solvate thereof of any one of claims 1-10, wherein the antibody or antigen-binding fragment thereof comprises two pairs of heavy chains and light chains, wherein the two heavy chains can be the same or different, and the two light chains can be the same or different, e.g., wherein the heavy chain and the light chain contain the following combinations of mutations:

| Combinations | First | First | Second | Second |
|---|---|---|---|---|
| of mutation | heavy chain | light chain | heavy chain | light chain |
| i | F126C/C220S | Q124C/C214S | F126C/C220S | Q124C/C214S |
| ii | H168C/C220S | T164C/C214S | H168C/C220S | T164C/C214S |
| iii | F170C/C220S | T164C/C214S | F170C/C220S | T164C/C214S |
| iv | F170C/C220S | S162C/C214S | F170C/C220S | S162C/C214S |
| v | V173C/C220S | S162C/C214S | V173C/C220S | S162C/C214S |
| vi | S134C/C220S | F116C/C214S | S134C/C220S | F116C/C214S |
| vii | F126C/C220S | Q124C/C214S | / | / |
| viii | K133C/C220S | I117C/C214S | K133C/C220S | I117C/C214S |
| ix | K133C/C220S | F209C/C214S | K133C/C220S | F209C/C214S |
| x | A141C/C220S | F116C/C214S | A141C/C220S | F116C/C214S |
| xi | P130C/C220 | F118C/C214S | P130C/C220 | F118C/C214S |
| xii | F170C/C220S | T164C/C214S | / | / |
| xiii | F170C/C220S | S162C/C214S | / | / |
| xiv | S134C/C220S | F116C/C214S | / | / |
| xv | P130C/C220 | F118C/C214S | / | / |

12. The ADC or a pharmaceutically acceptable salt or solvate thereof of any one of claims 1-11, wherein the heavy chain constant region is a constant region of human IgG1, IgG2, IgG3, or IgG4, for example, a constant region of IgG1;
the light chain constant region is a Kappa or Lambda constant region; or
CH1 is a CH1 region of human IgG1, IgG2, IgG3 or IgG4.

13. The ADC or a pharmaceutically acceptable salt thereof of any one of claims 1-12, wherein the antibody or antigen-binding fragment thereof is a human or humanized or chimeric antibody or antigen-binding fragment thereof, a monoclonal antibody, a Fab fragment, a single-arm antibody and/or multispecific antibody (e.g., a bispecific antibody), e.g., the antibody or antigen-binding fragment thereof specifically binds to HER2, B7H3 and/or TROP2.

14. The ADC or a pharmaceutically acceptable salt or solvate thereof of any one of claims 1-13, wherein the antibody or antigen-binding fragment thereof comprises a fragment crystallizable region (Fc region), optionally, the Fc region is a Fc region of human IgG1, IgG2, IgG3 or IgG4, for example, with the SI mutations, LALA mutations, N297A mutations, YTE mutations, and/or FLAA mutations.

15. The ADC or a pharmaceutically acceptable salt or solvate thereof of any one of claims 1-14, wherein the ADC comprises a payload linked to an antibody or antigen-binding fragment thereof via a linker, the payload is covalently linked to the antibody or antigen-binding fragment thereof via a thiol-based conjugation, for example, the payload is a cytotoxic agent (e.g., Dxd or MMAE), a cytostatic agent, biologically active proteins, synthetic polymers, enzymes, nucleic acids (e.g., DNA, or RNA) and fragments thereof.

16. The ADC or a pharmaceutically acceptable salt or solvate thereof of any one of claims 1-14, wherein the ADC comprises a payload linked to an antibody or an antigen-binding fragment thereof via a linker, wherein the linker is linked to the antibody via a thiol-SH of an opened disulfide bond, for example, the linker contains a maleimide moiety.

17. The ADC or a pharmaceutically acceptable salt or solvate thereof of any one of claims 1-14, wherein the ADC comprises a payload linked to an antibody or antigen-binding fragment thereof via a linker, wherein the linker-payload is selected from mc-GGFG-Dxd or mc-VC-PAB-MMAE.

18. The ADC of any one of claims 1-17, wherein the drug-to-antibody ratio (DAR) is between 3.74 and 4; or wherein the DAR is between 5.70 and 6.

19. A method of preparing an antibody-drug conjugate (ADC) or a pharmaceutically acceptable salt or solvate thereof, comprising the following steps:
(a) incubating the antibody or antigen-binding fragment thereof of any one of claims 1-14 with a reducing agent to reduce native disulfide bonds within the antibody or antigen-binding fragment thereof to generate reduced thiol groups;
(b) introducing an excess amount of a linker-payload having a reactive group to react with the reduced thiol groups generated in step (a),
optionally, further comprising: (c) adding an effective amount of N-acetylcysteine to quench the excess payload, and then recovering the resultant antibody-drug conjugate;
optionally, the linker-payload comprises a maleimide group;
optionally, the linker-payload is linked to the antibody or antigen-binding fragment thereof via a thiol-based conjugation.

20. The method of claim 19, wherein at least 50%, 60%, 70%, 80%, or 90% of the resulting ADC products have a drug-to-antibody ratio (DAR) of about 4; or wherein at least 50%, 60%, 70%, 80%, or 90% of the resulting ADC products have a drug-to-antibody ratio (DAR) of about 6.

21. A method of treating a subject having cancer, the method comprising administering a therapeutically effective amount of a composition comprising the ADC of any one of claims 15-18 or a pharmaceutically acceptable salt or solvate thereof, to the subject, for example, the subject has a solid tumor cancer, for example, the cancer is breast cancer, lung cancer, pancreatic cancer, melanoma, oral cancer, mesothelioma, ovarian cancer, colorectal cancer, gastric cancer, cervical cancer, brain cancer, skin cancer, multiple myeloma, lymphoma, epithelial neoplasms, soft tissue sarcoma, esophageal cancers, or CNS tumors.

22. A method of decreasing the rate of tumor growth, the method comprising contacting a tumor cell with an effective amount of a composition comprising the ADC or a pharmaceutically acceptable salt or solvate thereof of any one of claims 15-18.

23. A pharmaceutical composition comprising the ADC or a pharmaceutically acceptable salt or solvate thereof of any one of claims 1-18, and a pharmaceutically acceptable carrier.
